(19) United States
(12) Patent Application Publication
(10) Pub. No.: US 2018/0043037 A1
(43) Pub. Date: Feb. 15, 2018

(54) TISSUE SPECIFIC MARKERS FOR PREOPERATIVE AND INTRAOPERATIVE LOCALIZATION AND VISUALIZATION OF TISSUE

(71) Applicant: Invuity, Inc., San Francisco, CA (US)

(72) Inventors: Dennise DALMA-WEISZHAUSZ, Palo Alto, CA (US); Alex VAYSER, Mission Viejo, CA (US); James BREDENKAMP, Mission Viejo, CA (US)

(21) Appl. No.: 15/675,476

(22) Filed: Aug. 11, 2017

Related U.S. Application Data
(60) Provisional application No. 62/374,213, filed on Aug. 12, 2016.
(60) Provisional application No. 62/528,006, filed on Jun. 30, 2017.

(51) Int. Cl. A61K 49/00; C12N 15/115

(52) U.S. Cl. CPC A61K 49/006; C12N 15/115; A61K 49/0054; C12N 2310/16; C12N 2310/351; C12N 2310/335; C12N 2310/3517; A61B 5/4887

(57) ABSTRACT

Surgeons may face the difficulty of preoperatively selecting the location for making an incision and intraoperatively identifying and differentiating targeted tissue for removal or for identification so that it is not removed along with neighboring or non-target tissue or so that neighboring or non-target tissue is not nicked, harmed, or removed accidentally. A tissue specific marker can include an aptamer or an affimer configured to bind to a pre-selected target tissue and one or more indicator elements coupled with the aptamer or the affimer. The one or more indicator elements produce a signal thereby allowing identification of the target tissue in a preoperative and operative manner.

### CROSS-REFERENCE TO RELATED APPLICATIONS

This application claims priority to U.S. Provisional Application Ser. No. 62/374,213 filed on Aug. 12, 2016 and U.S. Provisional Application Ser. No. 62/528,006, filed Jun. 30, 2017, each of which application is incorporated herein by reference in its entirety.

### BACKGROUND

One of the challenges surgeons face while operating on a patient is differentiating target tissue from neighboring tissues within the surgical field. Without affordable and accessible tools, surgeons must rely solely on their skill and experience to make decisions when selecting an incision site and separating and selecting tissue for removal. It would be of great benefit for surgeons to be able to easily mark a specific tissue, either for removal or for the purpose of clear identification so that small glands, ducts or difficult to distinguish tissues are not unintentionally removed or damaged.

For example, when a patient has hyperthyroidism, goiter or thyroid cancer, an endocrine or a head and neck surgeon will usually perform a thyroidectomy. A thyroidectomy is an operation that removes at least part of the thyroid gland, a butterfly-shaped gland located at the base of the neck. One of the most common complications of thyroidectomy, is hypocalcemia or hypoparathyroidism. Calcium regulation in the body is managed by a group of small bean-sized glands called the parathyroids. Hypocalcemia or hypoparathyroidism after a thyroidectomy occurs because of the accidental removal of the parathyroid glands during the thyroidectomy. This is a challenge for surgeons because the parathyroids are small glands whose location varies from patient to patient, making it difficult to dissect only the portion of the thyroid that is affected without accidentally removing the small parathyroid glands that are in close proximity. Enabling the surgeon to easily identify the location of the parathyroids relative to the altered or affected thyroid tissue would allow them to do a more precise thyroid-only dissection sparing the small parathyroid glands.

Another example is parathyroidectomy, the surgical removal of at least one of the parathyroid glands, which is the most common and effective treatment for hyperparathyroidism, a condition that is caused by a benign tumor (parathyroid adenoma) or an enlargement (hyperplasia) of the parathyroid tissue. Enlargement of these glands results in overproduction of Parathyroid Hormone (PTH), which causes an increase in blood calcium, which in turn causes other serious symptoms including fragile bones, kidney stones, osteoporosis, hypertension, weakness, depression, etc. When the surgeon removes the affected gland(s), the PTH level will usually return to normal. In parathyroidectomies, the surgeon must decide where to make an incision and once the incision is made, he must also differentiate the parathyroid tissue from neighboring tissues. This is challenging because the parathyroid glands are small and difficult to locate among other structures in the neck such as thyroid, lymph nodes, etc. The exact location of these small glands may also vary from patient to patient. Additionally, the patients, predominantly females between 40 and 70 years of age, may not want significant scarring on their neck, which is an often exposed part of the body; as such, surgeons may want to make the smallest and fewest possible incisions. Precisely locating these small glands before selecting surgical incision sites may enable surgeons to reduce the number and size of incisions and by extension, the discomfort of post-surgery recovery and scarring. Current strategies for the preoperative identification of the parathyroid (e.g., adenoma) gland location include Sestamibi (99-Technetium) scans, ultrasound and in some cases computed tomography (CT) scans, but these are costly and not perfectly reliable. Affordable and precise tools and methods that allow surgeons to preoperatively identify the location, size and health of the glands and intraoperatively differentiate the tissues from neighboring tissue may assist surgeons in performing surgical procedures with minimal disruption to the patient's external and internal tissues. It would therefore be desirable to provide improved techniques that address some of the aforementioned challenges. Some of these objectives will be met by the methods and compositions described in this application.

### Background References

Scientific Articles: Smith, B., Gambir, S. Selective uptake of single-walled carbon nanotubes by circulating monocytes for enhanced tumor delivery. Nature Nanotech. 9, 481-487 (2014); Xiong, L., Rao, J. Self-luminescing BRET-FRET near infrared dots for in vivo lymph node mapping and tumor imaging. Nat. Commun. 3, 1-15 (2012); Lee, J., Rao, J. Combining SELEX Screening and Rational Design to Develop Light-up Fluorophore-RNA Aptamer Pairs for RNA Tagging. ACS Chem. Biol. 19, 1065-1074 (2010); Chapman, S., Rao, J. Nanoparticles for cancer imaging: the good, the bad and the promise Nano Today. 8, 454-460.

Grants: Samuel Achilefu. Development of Google System for Fluorescence Image-Guided Surgery, 5R01CA171651-03; Dustin Wayne Demoin. Phlip-based agents for pre-; intra-; and post-operative imaging and therapy of br. 1F32CA186721-01A1; Robert Hitchcock. Fiber-optic confocal imaging for ID of conduction tissue during cardiac surgery. 5R21HL108099-02; Aaron M Mohs. Nanotechnology for minimally invasive cancer detection and resection. 5R00CA153916-05; Quyen Nguyen. Testing fluorescently labelled probes for nerve imaging during surgery. 5R01EB014929-03; Brian W. Pogue and Keith Paulsen. Molecular fluorescence-guided surgery platform. 5R01CA167413-02; Marcin Ptaszek. Novel activatable fluorophores for multicolor fluorescence-guided cancer surgery. 1U01CA181628-01; Brian Straight. Intraoperative assessment of non-melanoma skin cancer margins using NIRF probes. 1R43CA180296-01A1; Ralph Weissleder. Novel Clickdyes for biomedical sensing. 2R01EB010011-05; Steven A. Benner. Simple inexpensive assay for five common HIV resistance mutations. 1R41AI116445-01; Steven A. Benner. Expanded DNA; in vitro selection; aptamers; and cancer. 1R01GM111386-01; Kyung Kang. Highly specific and highly sensitive aptamer-gold nanoparticle based NIR contrast. 1R1CA173693-01; Martin Schnermann. New synthetic approaches to small molecules for imaging near-IR photorelease chemistry: discovery and applications. 1ZIABC011506-02; Martin Schnermann. Near-IR photorelease chemistry: discovery and applications. 1ZIABC011564-01; Weihong Tan. Development of molecular probes for biomedical applications. 5R01GM079359-06.

Patents/Patent Publications: U.S. Pat. No. 8,685,372, US2014/0140594, US2014/0276008, CA2,611,468, CA2,770,980, and US2013/0134922.

### SUMMARY OF THE INVENTION

The claimed invention, in some aspects, relates to surgical tools and methods, and in some more particular aspects relates to tissue specific markers that facilitate the identification of target tissue from adjacent tissue, or a system which includes the marker and methods of use of the marker or markers.

In an aspect, a tissue specific marker comprises an aptamer or an affimer configured to bind to a pre-selected target tissue, and one or more indicator elements coupled to the aptamer or the affimer. The one or more indicator elements may produce a signal including spectral, paramagnetic, acoustic, etc. thereby allowing identification of the target tissue.

In another aspect, a tissue specific marker comprises an aptamer or an affimer configured to selectively bind to a non-malignant target tissue and/or a normal tissue; and at least a first indicator element coupled to the aptamer or the affimer, wherein the at least a first indicator element produces a signal, thereby allowing identification of the non-malignant target tissue. In some embodiments, the tissue specific marker comprises the aptamer configured to selectively bind to the non-malignant target tissue (and/or the normal tissue), wherein the aptamer comprises DNA, RNA, a peptide, or any combination thereof.

In some embodiments, the aptamer comprises a nucleic acid, peptides, DNA, RNA, or modified nucleotides or nucleosides. In some embodiments, the aptamer or the affimer is PEGylated or otherwise altered for increased stability. In some embodiments, the aptamer comprises RNA or DNA, or both RNA and DNA. In some embodiments, the aptamer is an RNA aptamer. In some embodiments, the aptamer is a DNA aptamer. In some embodiments, the aptamer comprises an inverted T at a 3′ end of the aptamer, e.g., an inverted Tat a 3′ end of a nucleic acid aptamer. In some embodiments, the aptamer has a length of up to 100 nucleotides.

In some embodiments, the non-malignant target tissue comprises a parathyroid gland or a parathyroid adenoma. In some embodiments, the non-malignant target tissue comprises a parathyroid. In some embodiments, the non-malignant target tissue is a human tissue. In some embodiments, the non-malignant target tissue is a non-human tissue. In some embodiments, the non-malignant target tissue is a gland tissue. In some embodiments, the gland tissue is an exocrine gland tissue or an endocrine gland tissue.

In some embodiments, the target tissue is a healthy tissue, normal tissue and/or non-malignant tissue. In some embodiments, the non-malignant tissue is a non-adipose, healthy tissue. In some embodiments, the non-malignant tissue is a healthy tissue that is not adipose tissue. In some embodiments, the non-malignant tissue is not a healthy tissue. In some embodiments, the signal produced can be within the magnetic, acoustic, visible, near-infrared, and infrared spectra. In some embodiments, the aptamer binds to the non-malignant target tissue with an affinity within a range of 1 pM to 1 mM. In some embodiments, the aptamer selectively binds to the target tissue with an affinity at least 10-fold higher than an affinity of the aptamer binding to a non-target tissue. In some embodiments, the aptamer selectively binds to the non-malignant target tissue with an affinity at least 2-fold higher than an affinity of the aptamer binding to a non-target tissue. In some embodiments, the non-target tissue comprises a thyroid. In some embodiments, the non-malignant target tissue is a non-human tissue. In some embodiments, the target tissue comprises a parathyroid gland or a parathyroid adenoma. In some embodiments, the non-malignant target tissue comprises a parathyroid. In some embodiments, the non-malignant target tissue comprises a nerve, a blood vessel, a ureter, a bile duct, endometrial tissue, hepatic duct, lymph nodes, bacteria or fungus.

In some embodiments, the aptamer or the affimer is configured to selectively bind to a first gland over a second gland. In some embodiments, the aptamer or the affimer is configured to preferentially bind the non-malignant target tissue over a tissue situated adjacent to the non-malignant target tissue.

In some embodiments, the aptamer or the affimer is configured to preferentially bind the gland tissue over at least one tissue selected from the group consisting of adipose, thymus, lymph node and pharynx tissue. In some embodiments, the aptamer or the affimer is configured to selectively bind to a healthy parathyroid tissue or a parathyroid adenoma tissue. In some embodiments, the aptamer or the affimer is configured to selectively bind to both the healthy parathyroid tissue and the parathyroid adenoma tissue. In some embodiments, the aptamer or the affimer preferentially binds to the healthy parathyroid tissue or the parathyroid adenoma tissue over a thyroid tissue. In some embodiments, the aptamer or the affimer preferentially binds to the healthy parathyroid tissue and the parathyroid adenoma tissue over the thyroid tissue. In some embodiments, the aptamer or the affimer preferentially binds to the healthy parathyroid tissue or the parathyroid adenoma tissue over at least one tissue selected from the group consisting of adipose, thymus, lymph node and pharynx tissue.

In some embodiments, the non-malignant target tissue comprises a nerve, a blood vessel, a ureter, a bile duct, endometrial tissue, hepatic duct, lymph nodes, bacteria or fungus. In some embodiments, the non-malignant target tissue comprises female reproductive tissues such as endometrium or uterus.

In some embodiments, the aptamer comprises a sequence with at least 70%, at least 75%, at least 80%, at least 85%, at least 90%, at least 95%, at least 98%, at least 99%, or 100% sequence identity to any one of SEQ ID NOs: 1-200. In some embodiments, the aptamer comprises a sequence with at least 70%, at least 75%, at least 80%, at least 85%, at least 90%, at least 95%, at least 98%, at least 99%, or 100% sequence identity to any one of SEQ ID NOs: 1-10 and SEQ ID NOs: 100-110. In some embodiments, the aptamer comprises a sequence with at least 70%, at least 75%, at least 80%, at least 85%, at least 90%, at least 95%, at least 98%, at least 99%, or 100% sequence identity to SEQ ID NO:1, SEQ ID NO:2, SEQ ID NO:3, SEQ ID NO:4, SEQ ID NO:5, SEQ ID NO:101, SEQ ID NO:102, SEQ ID NO:103, SEQ ID NO:104, or SEQ ID NO:105. In some embodiments, the aptamer comprises a sequence with at least 70%, at least 75%, at least 80%, at least 85%, at least 90%, at least 95%, at least 98%, at least 99%, or 100% sequence identity to SEQ ID NO:3, SEQ ID NO:4, SEQ ID NO:103, or SEQ ID NO:104. In some embodiments, the aptamer comprises a sequence of SEQ ID NO:3, SEQ ID NO:4, SEQ ID NO:103, or SEQ ID NO:104. In some embodiments, the aptamer comprises a sequence that includes the motif GATACTG.

The target tissue may comprise, for example, a parathyroid gland or a parathyroid adenoma. The target tissue may also comprise an organ, a nerve, a blood vessel, a ureter, endometrium, the thyroid, a bile duct, a hepatic duct, lymph nodes, bacteria, fungus or malignant tissue. In some embodiments, the one or more indicator elements or the at least a first indicator produces the signal when exposed to or excited by energy, or may be detectable by any other known method. In some embodiments, the signal produced is within a magnetic, acoustic, visible, near-infrared, or infrared spectrum. In some embodiments, the at least a first indicator element comprises a fluorophore. In some embodiments, the fluorophore is near-infrared dye, a cyanine dye or indocyanine green.

In some embodiments, the one or more indicator elements or the at least a first indicator element comprise a fluorophore, a quantum dot, a dye, a nanodiamond, an enzyme, a protein, a nanocrystal, gold or iron oxide particles, an optoacoustic converter element that converts light or near infrared signal to an acoustic output, a nanoparticle, nanorod, bead, or combination thereof. In some embodiments, the at least a first indicator element is covalently coupled to the aptamer, the affimer, or a second indicator element. In some embodiments, the at least a first indicator element comprises a first indicator element that is a fluorophore and a second indicator element that is a nanodiamond.

In some embodiments, two or more tissue specific markers may be used, wherein the second tissue specific marker comprises a second aptamer or affimer configured to bind to a second pre-selected target tissue different than the first pre-selected target tissue. In some cases, the first pre-selected target tissue is parathyroid tissue and the second pre-selected target tissue is thyroid tissue. The one or more indicator elements may be coupled to an aptamer, an affimer, or one or more other indicator elements. In some cases, one of the indicator elements will be detectable at a deeper distance. This may allow the surgeon to identify a target tissue preoperatively (through the skin). As the surgeon starts his dissection, another type of indicator may be used to distinguish the tissue more precisely.

In another aspect, a system for differentiating target tissue from adjacent tissue comprises any of the aspects of the marker described herein, and a probe or device for exciting the marker with energy and/or a probe or detector for detecting the spectral signal from the one or more indicator elements. The excitation device (or probe) and the detector (or probe) may be the same device (or probe), or they may be different devices (probes).

In an aspect, a system for differentiating a target tissue from an adjacent tissue is disclosed. The system comprises the tissue specific marker as described herein and a device for exciting the tissue specific marker with energy and/or a detector for detecting the signal from the one or more indicator elements. In some embodiments, the device for exciting the tissue specific marker with energy is an illumination source. In some embodiments, the detector comprises a camera.

In another aspect, a system for differentiating a target tissue from an adjacent tissue is disclosed. The system may comprise the tissue specific marker as described herein, a second tissue specific marker, comprising a second aptamer or a second affimer configured to bind to a second target tissue and a second indicator element coupled with the second aptamer or the second affimer, wherein the second indicator element produces a signal thereby allowing identification of the second target tissue and a first device for exciting the tissue specific marker with energy or a detector for detecting the signal from any of the first or second indicator elements, or from both the first and second indicator elements. In some embodiments, the system comprises an illumination source. In some embodiments, the system comprises a camera.

In some embodiments, a non-malignant target tissue is a parathyroid tissue and the first indicator element is a first fluorophore and wherein the second target tissue is nerve, lymph node, or thyroid tissue and the second indicator element is a second fluorophore different from the first fluorophore.

In another aspect, a method for differentiating tissue comprises delivering an aptamer or an affimer coupled to the one or more indicator elements into a patient's or a subject's body, allowing the aptamer or the affimer to bind to a target tissue, a normal or a non-malignant target tissue, detecting a signal produced by the one or more indicator elements or detecting the one or more indicator elements, and identifying the target tissue(s) from adjacent tissue based on the detected signal or distinguishing the normal or non-malignant target tissue from an adjacent tissue based on the signal detected from the one or more indicator elements. In some embodiments, the method comprises exposing or exciting the one or more indicator elements that are coupled to the aptamer or the affimer with energy.

In some embodiments, the method comprises delivering the aptamer or the affimer or the tissue specific marker comprises transdermally delivering, spraying, flooding, orally delivering, or intravenously delivering the aptamer or the affimer to the non-malignant target tissue. The method further comprises performing a medical procedure on the target tissue without damaging adjacent tissue. For example, the method may comprise removing at least a portion of the thyroid gland without damaging adjacent normal parathyroid tissues. Alternatively, the target tissue may comprise one or more abnormal (e.g., adenoma, hyperplasia or malignant tumor) parathyroid glands during a parathyroidectomy.

In some embodiments, the method comprises delivering the aptamer or the affimer comprises a washing step. In some embodiments, the method further comprises performing a medical procedure on the adjacent tissue without damaging the non-malignant target tissue, or without significantly damaging the non-malignant target tissue. In some embodiments, the method comprises performing a medical procedure on the non-malignant target tissue without damaging the adjacent tissue. In some embodiments, the non-malignant target tissue is a parathyroid adenoma and the method further comprises removing at least a portion of the parathyroid adenoma without damaging an adjacent thyroid tissue. In some embodiments, the non-malignant target tissue is a parathyroid tissue, and the method further comprises removing at least a portion of the adjacent thyroid tissue without damaging or removing the parathyroid tissue.

In some embodiments, the aptamer or the affimer selectively binds to a healthy parathyroid tissue or a parathyroid adenoma tissue. In some embodiments, the aptamer or the affimer selectively binds to both the healthy parathyroid tissue and the parathyroid adenoma tissue. In some embodiments, the aptamer or the affimer selectively binds to both the healthy parathyroid tissue and the parathyroid adenoma tissue over the adjacent thyroid tissue.

In some embodiments, the method comprises detecting a first set of the one or more indicator elements and a second set of the one or more indicator elements, wherein the first set is detected at a greater distance from the tissue specific marker than the second set. In some embodiments, the first set is detected outside the subject's body and the second set is detected after a surgical incision is made.

In some embodiments, identifying the non-malignant target tissue comprises visualizing the signal from the one or more indicator elements. In some embodiments, detecting the signal from the one or more indicator elements comprises detecting a spectral signal with a detector. In some embodiments, detecting the signal from the one or more indicator elements comprises detecting a spectral signal with a camera. In some embodiments, the method further comprising forming a diagnosis based on the detected spectral signal. In some embodiments, detecting the signal from the one or more indicator elements comprises detecting the signal preoperatively. In some embodiments, detecting the signal from the one or more indicator elements comprises detecting the signal intraoperatively.

The method may comprise the detection of at least two different indicator elements. In some embodiments, at least two different indicator elements are the same type of indicator element such as a fluorophore and are distinguishable by color or emission wavelength. In some embodiments, the two different indicator elements are different types of indicator elements. For example, a first indicator element may be an Indocyanine Green (ICG) fluorophore or a pH sensitive indicator and the second indicator element may be a nanodiamond. In some embodiments, a first indicator element produces a signal that is detectable when the probe is placed at a first distance from the labeled tissue and a second indicator element produces a signal that is detectable at a second distance that is greater than the first distance. In some embodiments, a first indicator element may be detected after a surgical incision is made and a second indicator element may be detected outside the body (e.g., through the skin). In some embodiments, the first and second indicator elements produce different types of signals.

In some embodiments, the method comprises detecting the signal from the one or more indicator elements using a probe or other device. In one instance, identifying the target tissue may comprise visualizing a signal emitted from the one or more indicator elements. Detecting the signal may comprise detecting the one or more indicator elements with a camera, for example a complementary metal-oxide-semiconductor (CMOS) camera or a charge coupled display (CCD) camera. Detecting the signal from the one or more indicator elements may be used diagnostically, or non-diagnostically, preoperatively, intraoperatively or in any combination of these applications.

In an aspect, provided herein is a tissue specific marker, said marker comprising: an aptamer or an affimer configured to bind to a pre-selected target tissue; and at least a first indicator element coupled to the aptamer or the affimer, wherein the at least a first indicator element produces a signal allowing identification of the target tissue. In some embodiments, the aptamer comprises DNA, RNA, or a peptide, or any combination thereof. In some embodiments, the aptamer comprises modified nucleotides or nucleosides. In some embodiments, the aptamer comprises a sequence with at least 70%, at least 75%, at least 80%, at least 85%, at least 90%, at least 95%, at least 98%, at least 99%, or 100% sequence identity to any one of SEQ ID NOs: 1-200. In some embodiments, the aptamer comprises a sequence with at least 70%, at least 75%, at least 80%, at least 85%, at least 90%, at least 95%, at least 98%, at least 99%, or 100% sequence identity to any one of SEQ ID NOs: 1-10 and SEQ ID NOs: 100-110. In some embodiments, the aptamer comprises a sequence with at least 70%, at least 75%, at least 80%, at least 85%, at least 90%, at least 95%, at least 98%, at least 99%, or 100% sequence identity to SEQ ID NO:1, SEQ ID NO:2, SEQ ID NO:3, SEQ ID NO:4, SEQ ID NO:5, SEQ ID NO:101, SEQ ID NO:102, SEQ ID NO:103, SEQ ID NO:104, or SEQ ID NO:105. In some embodiments, the aptamer comprises a sequence with at least 70%, at least 75%, at least 80%, at least 85%, at least 90%, at least 95%, at least 98%, at least 99%, or 100% sequence identity to SEQ ID NO:3, SEQ ID NO:4, SEQ ID NO:103, or SEQ ID NO:104. In some embodiments, the aptamer comprises a sequence of SEQ ID NO:3, SEQ ID NO:4, SEQ ID NO:103, or SEQ ID NO:104. In some embodiments, the aptamer comprises a sequence that includes the motif GATACTG. In some embodiments, the aptamer has a length of up to 50, 60, 70, 80, 90, 100, 110, 120, 130, 140, 150, or 200 nucleotides. In some embodiments, the aptamer binds to the target tissue with an affinity within a range of 1 pM to 1 mM, such as within a range of 1 nM to 100 μM.

In some embodiments, the target tissue is a healthy tissue, normal tissue and/or non-malignant tissue. In some embodiments, the non-malignant tissue is a non-adipose, healthy tissue. In some embodiments, the non-malignant tissue is a healthy tissue that is not adipose tissue. In some embodiments, the non-malignant tissue is not a healthy tissue. In some embodiments, the signal produced can be within the magnetic, acoustic, visible, near-infrared, and infrared spectra. In some embodiments, the aptamer selectively binds to the target tissue with an affinity at least 10-fold higher than an affinity of the aptamer binding to a non-target tissue. In some embodiments, the aptamer selectively binds to the non-malignant target tissue with an affinity at least 2-fold higher than an affinity of the aptamer binding to a non-target tissue. In some embodiments, the non-malignant target tissue comprises a parathyroid gland or a parathyroid adenoma. In some embodiments, the non-target tissue comprises a thyroid. In some embodiments, the non-malignant target tissue is a human tissue. In some embodiments, the non-malignant target tissue is a non-human tissue. In some embodiments, the target tissue comprises a parathyroid gland or a parathyroid adenoma. In some embodiments, the non-malignant target tissue comprises a nerve, a blood vessel, a ureter, a bile duct, endometrial tissue, hepatic duct, lymph nodes, bacteria or fungus.

In some embodiments, the at least a first indicator element comprises a fluorophore. In some embodiments, the fluorophore is a near-infrared dye. In some embodiments, the fluorophore is a cyanine dye. In some embodiments, the fluorophore is indocyanine green. In some embodiments, the at least a first indicator element comprises a quantum dot. In some embodiments, the at least a first indicator element comprises an enzyme or protein. In some embodiments, the at least a first indicator element comprises a pH sensitive indicator. In some cases, the at least a first indicator element comprises a nanodiamond. In some embodiments, the at least a first indicator element comprises an optoacoustic converter element. In some cases, the at least a first indicator element comprises a nanoparticle or nanorod. In some embodiments, the at least a first indicator element comprises a bead. In some embodiments, the at least a first indicator element is covalently coupled to the aptamer, the affimer, a second indicator element or the one or more indicator elements.

In another aspect, provided herein is a system for differentiating target tissue from adjacent tissue. The system further comprises a marker described herein and a probe for exciting the marker with energy or a probe for detecting the signal from the one or more indicator elements. In some embodiments, the system further comprises an illumination source. In some embodiments, the system further comprises a camera.

In another aspect, provided herein is a system for differentiating target tissue from adjacent tissue. The system further comprises a marker described herein, a second tissue specific marker, comprising a second aptamer or a second affimer configured to bind to a second pre-selected target tissue and one or more second indicator elements coupled with the second aptamer or the second affimer, wherein the second indicator element produces a spectral signal when exposed to or excited by energy thereby allowing identification of the second target tissue, a first probe for exciting the marker with energy or a probe for detecting the spectral signal from the one or more indicator elements and a second probe for exciting the marker with energy or a probe for detecting the spectral signal from the one or more second indicator elements. In some embodiments, the system further comprises an illumination source. In some embodiments, the system further comprises a camera.

In another aspect, provided herein is a method for differentiating tissue. The method comprises delivering an aptamer or an affimer coupled to one or more indicator elements into a patient's body, allowing the aptamer or the affimer to bind with a target tissue in the patient's body, exposing or exciting the one or more indicator elements with energy, detecting a signal produced by the one or more indicator elements or detecting the one or more indicator elements and distinguishing the target tissue from adjacent tissue based on the signal detected from the one or more indicator elements.

In another aspect, a method comprises delivering a second aptamer or a second affimer coupled to one or more indicator elements into a subject's body, allowing the second aptamer or the second affimer to bind with a second target tissue, wherein the second target tissue is different than a target tissue, exposing or exciting the one or more indicator elements that are coupled to the second aptamer or the second affimer with energy, detecting a spectral signal produced by the one or more indicator elements that are coupled to the second aptamer or the second affimer or detecting the one or more indicator elements that are coupled to the second aptamer or the second affimer and identifying the second target tissue from the target tissue based on the signal detected from the one or more indicator elements that are coupled to the second aptamer or the second affimer.

In some embodiments, delivering an aptamer or an affimer comprises transdermally delivering, spraying, flooding, orally delivering, or intravenously delivering the aptamer or the affimer to the target tissue. In some cases, delivering the aptamer or affimer comprises a washing step. In some cases, the method further comprises performing a medical procedure on the non-malignant target tissue without damaging adjacent tissue. In some cases, the target tissue is a thyroid gland, and the method further comprises removing at least a portion of the thyroid gland without damaging adjacent parathyroid tissue. In some cases, the target tissue is a parathyroid gland, and the method further comprises removing one or more parathyroid glands without damaging or removing adjacent thyroid.

In some embodiments, the method further comprises detecting a first set of the one or more indicator elements and a second set of the one or more indicator elements, wherein the first set is detected at a greater distance from the tissue specific marker than the second set. In some cases, the first set is detected outside the body and the second set is detected after a surgical incision is made.

In some embodiments, identifying the target tissue comprises visualizing the signal from the one or more indicator elements. In some cases, detecting the signal from the one or more indicator elements comprises detecting the signal with a probe. In some cases, detecting the signal from the one or more indicator elements comprises detecting the spectral signal with a camera. In some cases, the method further comprises forming a diagnosis based on the detected signal. In some cases, detecting the signal from the one or more indicator elements comprises detecting the signal preoperatively. In some cases, detecting the signal from the one or more indicator elements comprises detecting the signal intraoperatively.

In some embodiments, the method further comprises delivering a second aptamer or a second affimer coupled to one or more indicator elements into a patient's or the subject's body; allowing the second aptamer or the second affimer to bind with a second target tissue, wherein the second target tissue is different than the normal or non-malignant target tissue; exposing or exciting the one or more indicator elements that are coupled to the second aptamer or the second affimer; detecting a signal produced by the one or more indicator elements that are coupled to the second aptamer or the second affimer or detecting the one or more indicator elements that are coupled to the second aptamer or the second affimer; and identifying the second target tissue from the normal or non-malignant target tissue based on the signal detected from the one or more indicator elements that are coupled to the second aptamer or the second affimer.

In an aspect, provided herein is an aptamer that selectively binds to a parathyroid gland or a parathyroid adenoma. In some embodiments, the aptamer selectively binds to the parathyroid gland or the parathyroid adenoma over a thyroid gland. In some embodiments, the aptamer selectively binds to the parathyroid gland or the parathyroid adenoma with an affinity at least 10-fold higher than an affinity of the aptamer for binding to a thyroid gland. In some embodiments, the aptamer selectively binds to the parathyroid gland or the parathyroid adenoma with an affinity at least 5-fold, 10-fold, at least 20-fold, at least 50-fold, or at least 100-fold higher than an affinity of the aptamer binding to a thyroid gland. In some embodiments, the aptamer comprises DNA. In some embodiments, the aptamer comprises RNA. In some embodiments, the aptamer, the aptamer selectively binds to a normal, healthy, and/or non-malignant parathyroid gland. In some embodiments, the aptamer is configured to selectively bind to both healthy parathyroid tissue and parathyroid adenoma tissue. In some embodiments, the aptamer preferentially binds to healthy parathyroid tissue or parathyroid adenoma tissue over a thyroid tissue. In some embodiments, the aptamer preferentially binds to healthy parathyroid tissue and parathyroid adenoma tissue over a thyroid tissue. In some embodiments, the aptamer preferentially binds to healthy parathyroid tissue or parathyroid adenoma tissue over at least one tissue selected from the group consisting of adipose, thymus, lymph node and pharynx tissue. In some embodiments, the aptamer selectively binds to human tissue. In some embodiments, the aptamer is configured to selectively bind to parathyroid tissue over a different type of gland tissue. In some embodiments, the aptamer is configured to preferentially bind normal, healthy, or non-malignant target tissue over a tissue situated adjacent to the normal, healthy, or non-malignant target tissue.

In some embodiments, the aptamer comprises a sequence with at least 70%, at least 75%, at least 80%, at least 85%, at least 90%, at least 95%, at least 98%, at least 99%, or 100% sequence identity to any one of SEQ ID NOs: 1-200. In some embodiments, the aptamer comprises a sequence with at least 70%, at least 75%, at least 80%, at least 85%, at least 90%, at least 95%, at least 98%, at least 99%, or 100% sequence identity to any one of SEQ ID NOs: 1-10 and SEQ ID NOs: 100-110. In some embodiments, the aptamer comprises a sequence with at least 70%, at least 75%, at least 80%, at least 85%, at least 90%, at least 95%, at least 98%, at least 99%, or 100% sequence identity to SEQ ID NO:1, SEQ ID NO:2, SEQ ID NO:3, SEQ ID NO:4, SEQ ID NO:5, SEQ ID NO:101, SEQ ID NO:102, SEQ ID NO:103, SEQ ID NO:104, or SEQ ID NO:105. In some embodiments, the aptamer comprises a sequence with at least 70%, at least 75%, at least 80%, at least 85%, at least 90%, at least 95%, at least 98%, at least 99%, or 100% sequence identity to SEQ ID NO:3, SEQ ID NO:4, SEQ ID NO:103, or SEQ ID NO:104. In some cases, the aptamer comprises a sequence of SEQ ID NO:3, SEQ ID NO:4, SEQ ID NO:103, or SEQ ID NO:104. In some cases, the aptamer comprises a sequence of GATACTG.

In an aspect, provided herein is a polynucleotide comprising a sequence with at least 70%, at least 75%, at least 80%, at least 85%, at least 90%, at least 95%, at least 98%, at least 99%, or 100% sequence identity to any one of SEQ ID NOs: 1-200. In an aspect, a polynucleotide comprises a sequence with at least 70% sequence identity to any one of SEQ ID NOs: 1-200, wherein the sequence comprises a non-natural sequence of at least ten contiguous nucleotides or the sequence comprises at least one modified nucleotide.

In some embodiments, the polynucleotide comprises a sequence with at least 70%, at least 75%, at least 80%, at least 85%, at least 90%, at least 95%, at least 98%, at least 99%, or 100% sequence identity to any one of SEQ ID NOs: 1-10 and SEQ ID NOs: 100-110. In some embodiments, the polynucleotide comprises a sequence with at least 70%, at least 75%, at least 80%, at least 85%, at least 90%, at least 95%, at least 98%, at least 99%, or 100% sequence identity to SEQ ID NO:1, SEQ ID NO:2, SEQ ID NO:3, SEQ ID NO:4, SEQ ID NO:5, SEQ ID NO:101, SEQ ID NO:102, SEQ ID NO:103, SEQ ID NO:104, or SEQ ID NO:105. In some embodiments, the polynucleotide comprises a sequence with at least 70%, at least 75%, at least 80%, at least 85%, at least 90%, at least 95%, at least 98%, at least 99%, or 100% sequence identity to SEQ ID NO:3, SEQ ID NO:4, SEQ ID NO:103, or SEQ ID NO:104. In some cases, the polynucleotide comprises a sequence of SEQ ID NO:3, SEQ ID NO:4, SEQ ID NO:103, or SEQ ID NO:104. In some cases, the polynucleotide comprises a sequence of GATACTG.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the anterior view of the thyroid and parathyroid tissues and related anatomy.

FIG. 2 illustrates the liver, gallbladder and adjacent vasculature.

FIG. 3 illustrates the renal system and adjacent nerve tissue.

FIG. 4 illustrates the peripheral nerve system.

FIG. 5 illustrates the use of the markers to identify lymph nodes.

FIG. 6 illustrates the sinuses and the application of markers targeted to bacteria.

FIG. 7 illustrates the use of the probe in identifying cancer or any malignant tissue.

FIGS. 8A-8C illustrate exemplary configurations of a tissue specific marker.

FIG. 9 illustrates a flowchart for intraoperative use of the tissue specific marker.

FIGS. 10A-10F illustrate use of a tissue specific marker in parathyroidectomy and thyroidectomy.

FIG. 11 illustrates a flowchart for possible combination of preoperative and intraoperative use of the tissue specific marker.

FIGS. 12A-12B illustrate the use of a probe for diagnostic or preoperative detection of the tissue specific marker.

FIG. 13 illustrates an exemplary strategy for selecting a parathyroid-specific marker.

FIG. 14 illustrates the copy number for 10 sequences, plotted against the selection round.

FIG. 15 illustrates a sequence alignment of aptamer SEQ ID NO:103 and SEQ ID NO:104.

FIG. 16 shows the protocol for aptamer binding on tissue slides.

FIG. 17A-17G illustrates binding of aptamer SEQ ID NO:3 to normal parathyroid tissue.

FIGS. 18A-18G Illustrate binding of aptamer SEQ ID NO:4 to normal parathyroid tissue.

FIGS. 19A-19B illustrate binding results of aptamer SEQ ID NO:3 to normal thyroid tissue and binding of SEQ ID NO:4 to normal thyroid tissue.

FIGS. 20A and 20B illustrate binding results of aptamer SEQ ID NO:3 and aptamer SEQ ID NO:4 on parathyroid adenoma, respectively.

FIGS. 21A and 21B illustrate binding results of aptamer SEQ ID NO:3 and SEQ ID NO:4 on additional normal parathyroid tissues, respectively.

FIGS. 22A and 22B illustrate binding results of aptamer SEQ ID NO:3 and aptamer SEQ ID NO:4 to adipose tissue, respectively.

FIGS. 23A and 23B illustrate binding results of aptamer SEQ ID NO:3 and aptamer SEQ ID NO:4 to lymph node, respectively.

FIGS. 24A and 24B illustrate binding results of aptamer SEQ ID NO:3 and aptamer SEQ ID NO:4 to oropharynx tissue, respectively.

FIGS. 25A and 25B illustrate binding results of aptamer SEQ ID NO:3 and aptamer SEQ ID NO:4 to thymus tissue, respectively.

FIGS. 26A and 26B show the map of the different tissues on a tissue microarray slide. 26A shows a schematic of the localization of the tissues; FIG. 26B shows a picture of the actual microarray slide.

FIGS. 27A-27AH illustrate binding results of aptamer SEQ ID NO:3 on additional healthy human tissues (tissue microarray).

FIGS. 28A-28AG illustrate binding results of aptamer SEQ ID NO:4 on additional healthy human tissues (tissue microarray).

### DETAILED DESCRIPTION OF THE INVENTION

Specific embodiments of the tissue specific marker and method of use will now be described, at times with reference to the drawings. Nothing in this detailed description is intended to imply that any particular component, feature, or step is essential to the invention.

This disclosure provides tissue specific marker compositions that can be used to identify or mark a particular tissue (e.g., healthy parathyroid tissue) during preoperative and intraoperative surgical procedures. The identification of a specific tissue type during a surgical procedure may be particularly helpful for mitigating damage to or loss of healthy tissues and organs. In some preferred embodiments, the tissue specific marker is used to mark a specific healthy tissue (e.g., parathyroid tissue) in order to distinguish it from a different tissue type (e.g., thyroid tissue, adipose tissue) or diseased tissue (e.g., adenoma, hyperplasia or thyroid malignant tumor). In some cases, the tissue specific marker compositions are used to mark healthy tissue to be avoided or preserved during surgery. In other cases, the tissue specific marker compositions are used to mark diseased tissue or other target tissue for surgical removal. In some cases, multiple tissue specific markers may be used. For example, a first tissue specific marker may be used to mark one tissue type (e.g., healthy tissue) while a second tissue specific marker is used to mark a different tissue type (e.g., diseased tissue, tissue from a different organ). In some cases, a single tissue specific marker may contain a first targeting element linked to a second targeting element, wherein the first and second targeting elements are designed to mark different types of tissue, so that two tissues in close proximity to the target tissue can be highlighted.

While the exemplary embodiments are primarily directed at tissue differentiation of thyroid, parathyroid, or adjacent tissue, this is not intended to be limiting, and one of skill in the art will appreciate that the markers, methods, and systems described here may be used to differentiate any target tissue. The identified or targeted tissue may be organ tissue, particularly tissue from a solid organ or gland. In some preferred embodiments, the target tissue is parathyroid tissue, nerve tissue or reproductive tissue (e.g., tissue derived from cervix, ovary, endometrium or other female reproductive organ). In further preferred embodiments, the target tissue is a healthy parathyroid tissue, a non-malignant parathyroid tissue, a normal parathyroid tissue or a diseased parathyroid tissue such as parathyroid adenoma tissue, parathyroid hyperplasia tissue or tissue of a parathyroid malignant tumor tissue.

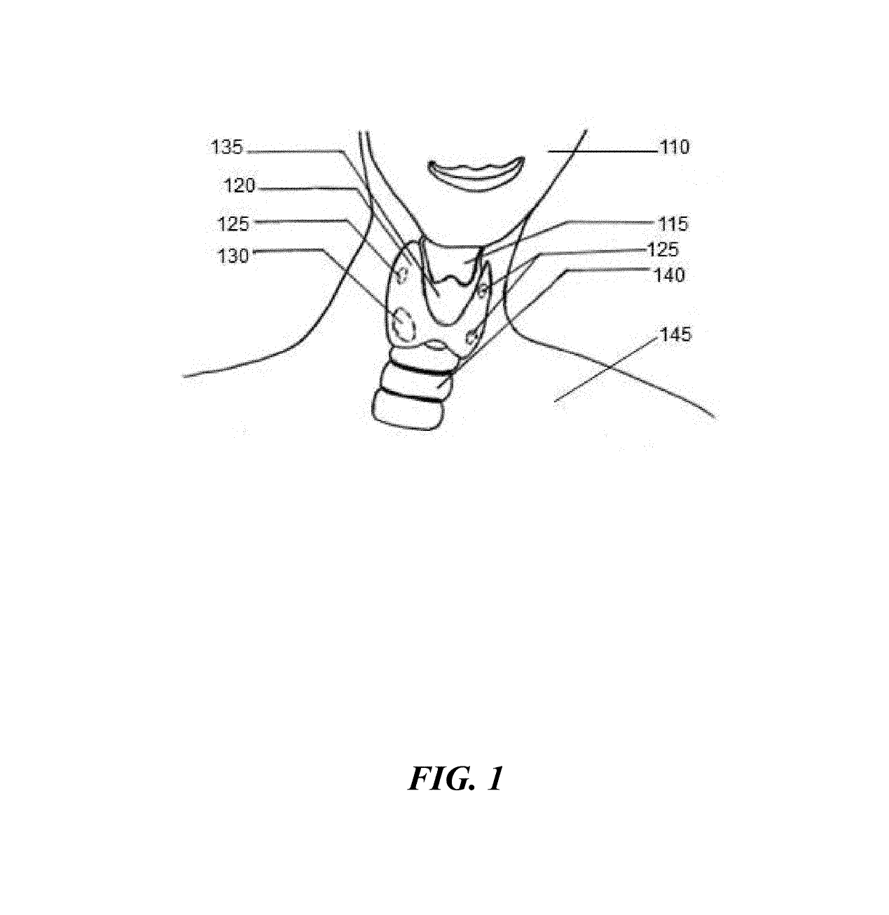

Many of the tissue specific marker compositions provided herein may be useful for surgical procedures involving complete or partial removal of the thyroid or parathyroid. FIG. 1 is a schematic diagram of the location of the thyroid 120 and parathyroid glands 125 from the anterior view with the head 110 and body 145 of the patient depicted for perspective. Healthy parathyroid glands 125 and diseased adenoma tissue 130 are shown relative to the larynx 115, the thyroid 120, the thyroid cartilage 135, and the trachea 140. The parathyroid glands 125 are small relative to the adjacent thyroid tissue 120, and there is significant patient-to-patient variability in the exact location of the parathyroid glands. These characteristics pose preoperative and intraoperative challenges to surgeons conducting parathyroidectomy and thyroidectomy procedures. Current approaches for preoperatively locating the healthy parathyroid glands 125 or parathyroid tissue adenoma 130, include Sestamibi (99-Technetium) scanning and ultrasound, which are costly and not perfectly reliable. Intraoperatively, the small size of the parathyroid glands 125 makes them difficult to locate relative to the thyroid gland 120; as a result, healthy parathyroid tissue may be undesirably excised or otherwise damaged during a thyroidectomy, and healthy thyroid tissue may be unnecessarily excised or damaged during a parathyroidectomy.

Tissue specific marker compositions provided herein may be used to mark or identify parathyroid tissue during a thyroidectomy, thereby preventing unnecessary excision or damage to parathyroid tissue during the procedure. In some cases, the tissue specific marker composition may be capable of identifying both diseased and healthy tissue. For example, a tissue specific marker may specifically bind both healthy and diseased parathyroid tissue (e.g., parathyroid adenoma tissue). Such tissue specific marker may also be useful during a parathyroidectomy in order to mark both the diseased parathyroid tissue (e.g., parathyroid adenoma tissue) and the healthy parathyroid tissue in order to avoid unnecessary removal or damage to thyroid tissue. In such cases, the diseased parathyroid tissue may be located preoperatively using a standard procedure such as a computed tomography (CT) scan, which may identify the quadrant in which the parathyroid adenoma or other diseased tissue is located. The surgeon may then make an incision in the general region in which the adenoma or diseased tissue is expected to be located and then use the tissue specific marker to more specifically identify the precise location of the adenoma tissue. Following the procedure, a blood test to detect parathyroid function such as a parathyroid hormone (PTH) test may be performed in order to provide further assurance that the adenoma tissue was removed. In still further embodiments, a patient may receive systemic administration of a tissue specific marker with a first indicator element and a second indicator element. The parathyroid tissue (diseased or healthy) may then be identified and located preoperatively using a device or probe that detects signal from the first indicator element above the skin, therefore avoiding the use of MRI or Sestamibi scan. This approach may give the surgeon a more accurate indication of the location of the parathyroid tissue so that the initial incision can be even more targeted or precise. Following the incision, the parathyroid may be visualized using the second indicator element. In some cases, the tissue specific marker contains a single indicator element that is detected preoperatively and/or during surgery. In some cases, the parathyroid-specific marker may be used with a thyroid-specific marker in order to further assist with differentiation between parathyroid and thyroid tissue. In still further cases, a thyroid-specific marker may be used alone or in conjunction with a parathyroid-specific marker.

The tissue specific marker compositions provided herein may be used to mark or identify parathyroid tissue (healthy and/or diseased), thereby preventing unnecessary excision or damage to parathyroid glands during a thyroidectomy procedure. In such cases, the surgeon may focus on removing unmarked thyroid tissue, rather than the marked parathyroid tissue. In some cases, the parathyroid-specific marker may be used with a thyroid-specific marker in order to further aid in the differentiation between parathyroid and thyroid tissue during a thyroidectomy. In still further cases, a thyroid-specific marker may be used singly or in conjunction with a parathyroid-specific marker.

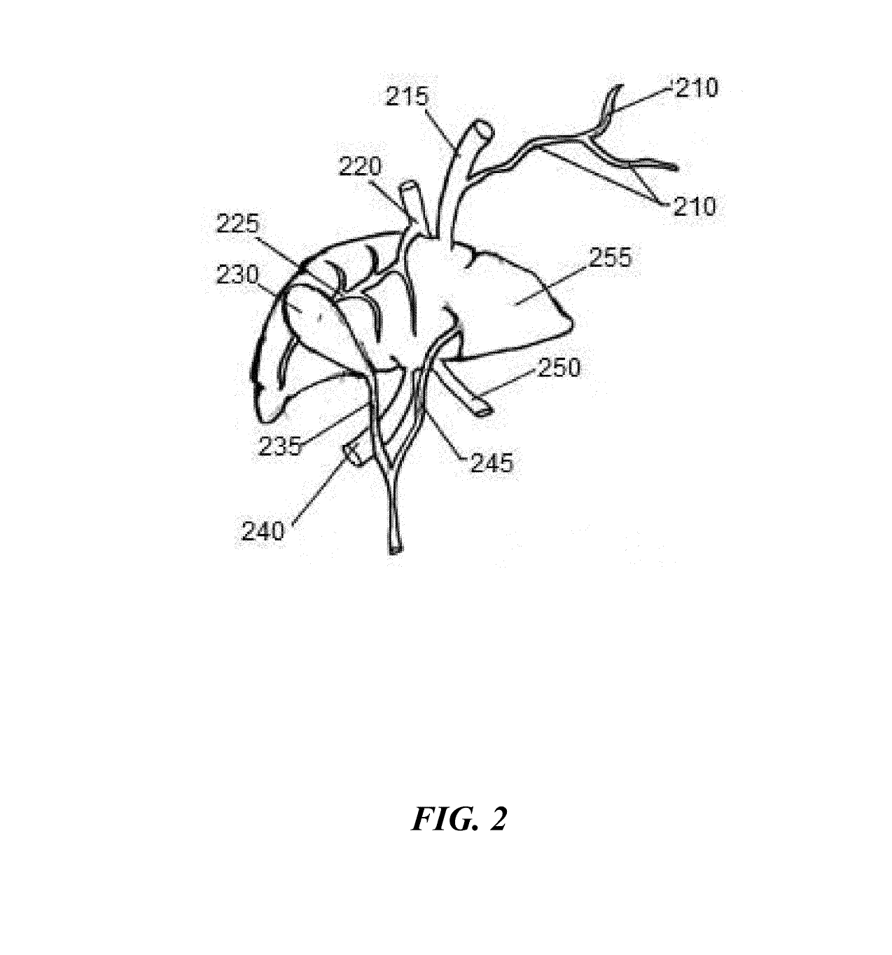

The compositions and methods provided herein may be used in any other surgical procedures involving target tissue that is difficult to distinguish from adjacent or neighboring tissue. FIGS. 2-7 illustrate examples of other regions of the body or tissues that may be operated on and which may benefit from the tissue specific markers and methods of use disclosed herein.

FIG. 2 is a schematic diagram showing the hepatic duct 245 and the bile duct 235 relative to adjacent vasculature 210, the aorta 215, inferior vena cava 220, minor hepatic arteries 225, the gall bladder 230, the portal vein 240, the hepatic artery 250, and the liver 255. A tissue specific marker may be applied in diagnostic, preoperative or intraoperative surgical procedures related to the hepatic duct and the bile duct as well as vasculature including vasculature in or around the depicted region of a patient's body. The tissue specific marker may be valuable for perfusion studies and may assist surgeons to avoid nicking blood vessels during surgery.

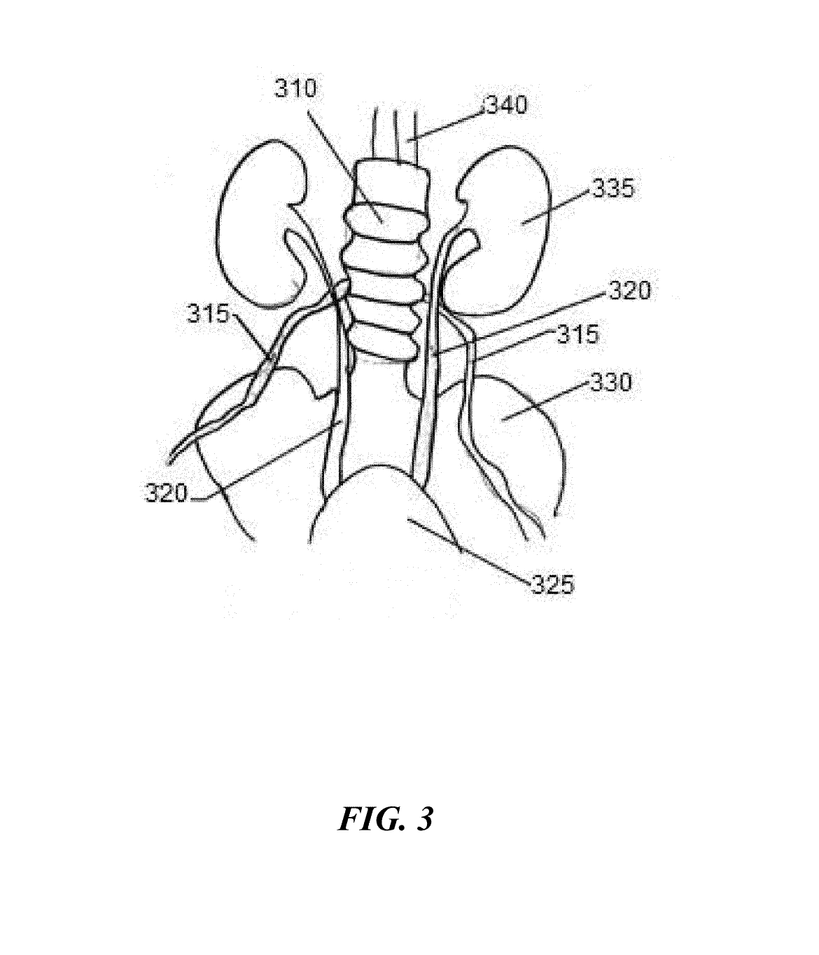

FIG. 3 is a schematic diagram showing the location of the ureters 320 relative to the spine 310, exemplary adjacent nerve tissue 315 originating from the spine 310, the bladder 325, pelvis 330, kidneys 335 and the spinal cord 340. The tissue specific marker may be applied in diagnostic, preoperative or intraoperative surgical procedures related to the ureters as well as nerve tissue including nerve tissue in or around the depicted region of a patient's body. The tissue specific marker can also be selected to bind to a non-solid target, for example one that goes through an anatomic lumen such as urine, and this may be another way for ureter identification and preservation.

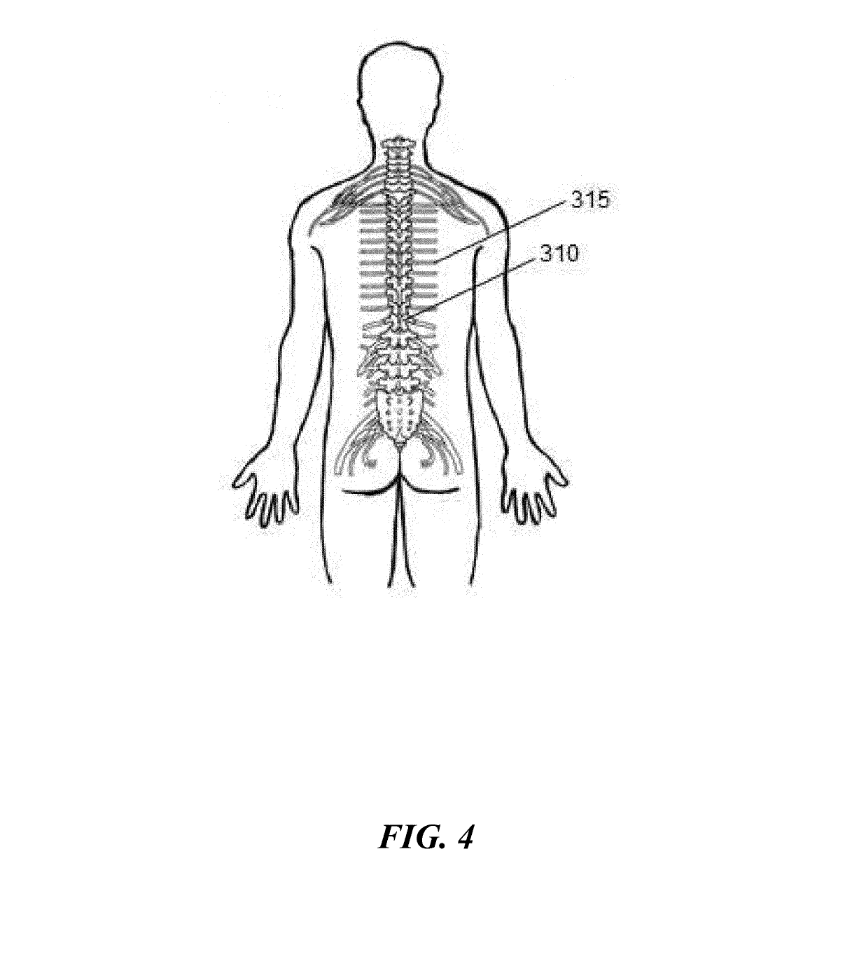

FIG. 4 is a schematic diagram showing the peripheral nerves 315, connected to the spine 310, in more detail, and tissue specific markers may be used to visualize either the CNS tissue or the peripheral nerves or both. During surgical procedures, for example a prostatectomy, tissue specific markers may be used to identify the nerves. Use of these tissue specific markers may allow a surgeon to see the location of one or more nerves during the operation thereby allowing better guidance and therefore providing a higher safety factor for a successful outcome, for example by preventing accidental nicking of a nerve.

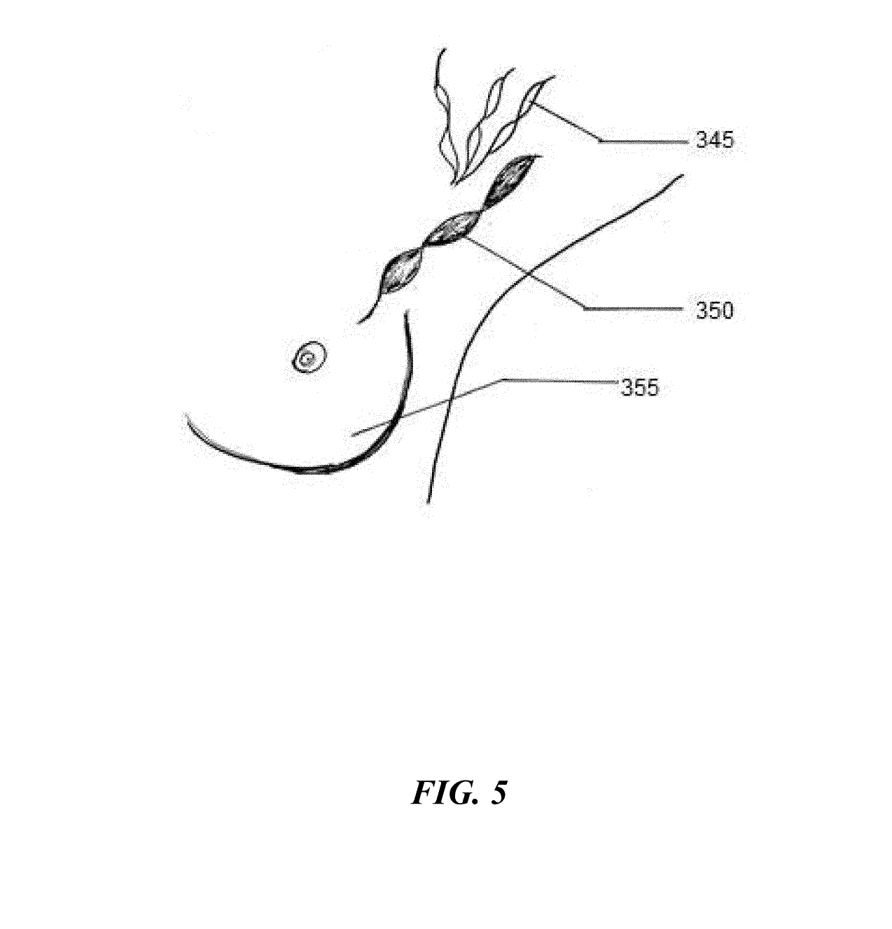

FIG. 5 is a schematic diagram showing another potential application of these tissue specific markers for distinguishing sentinel lymph nodes 350 or auxiliary lymph nodes 345 from adjacent tissue; the patient's breast 355 is shown for perspective. In this example the markers may be used for identifying the sentinel lymph node during surgical removal of cancerous breast tissue. Visualizing the sentinel lymph node reduces injury to the rest of the anatomy.

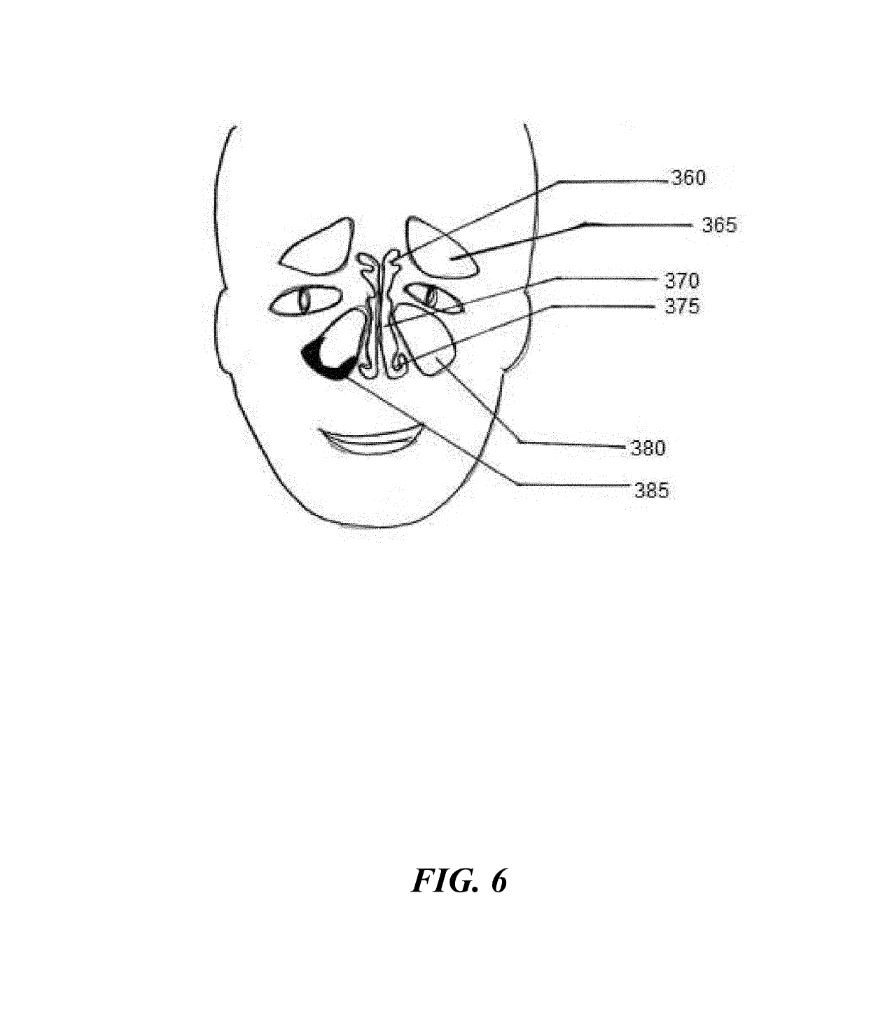

FIG. 6 is a schematic diagram showing the frontal sinus 365 and the maxillary sinus 380 relative to the superior 360, middle 370 and inferior 375 turbinate bones. During a sinus infection, bacteria may accumulate in the sinus 385, and cause a sinus infection. Tissue specific markers may also be comprised of aptamers that can bind to a bacteria or fungus and detect an infection site. These infectious-agent specific markers may be used for chronic sinusitis or wound care, and they may enable doctors or surgeons to image an infected region and facilitate patient specific treatment.

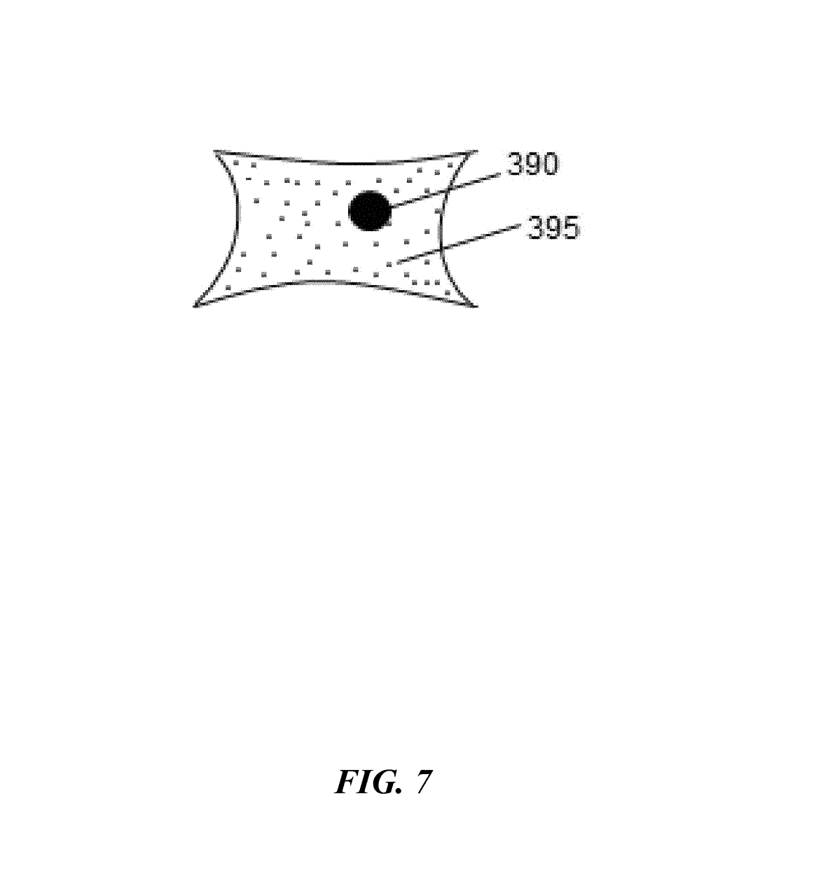

FIG. 7 is a schematic diagram showing how the tissue specific markers can target and bind to a tumor 390. The bound tissue specific markers may be detected and used to distinguish the tumor from healthy neighboring tissue 395.

Tissue Specific Markers

The tissue specific markers provided herein generally contain a targeting element linked to at least one indicator element. The tissue specific markers may contain other elements as well such as additional targeting elements, additional indicator elements, one or more PEGylated elements, one or more linkers, and/or one or more modified residues designed to reduce degradation of the markers in the blood or at body temperature. In general, these features may enable the tissue specific markers to have one or more of the following properties: (a) the ability to selectively bind a specific tissue type with relative high affinity compared to at least one other type of tissue; (b) the ability to be detected, particularly during or prior to a surgical procedure; and/or (c) a half-life of sufficient duration to permit the tissue specific markers to bind and localize to target tissue.

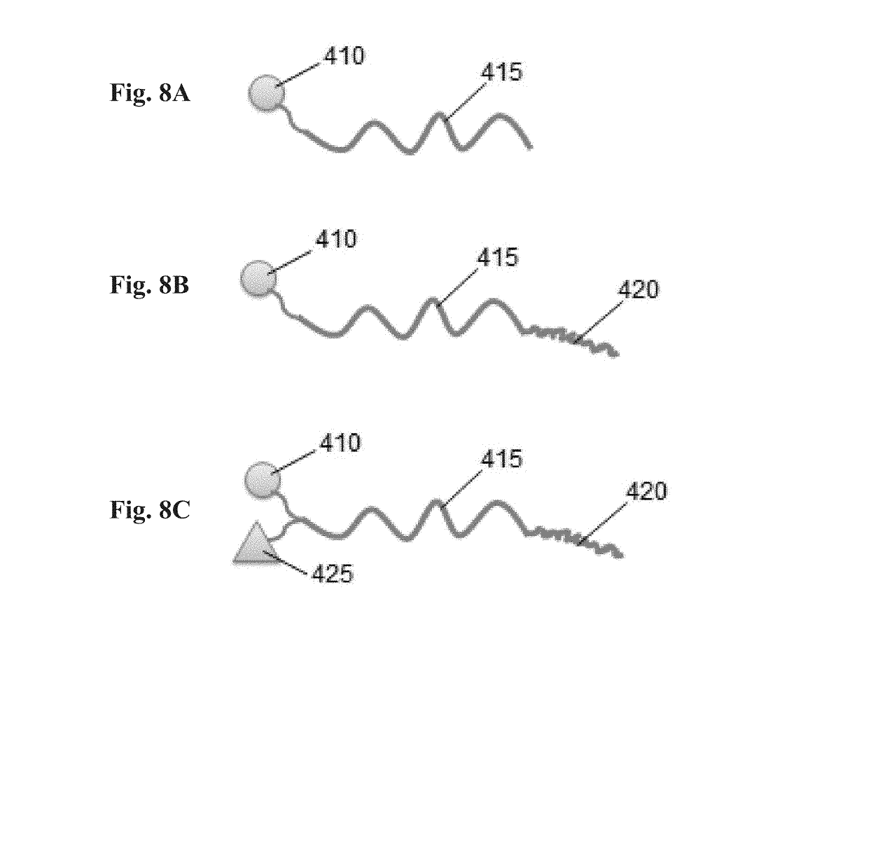

The tissue specific markers may have several variations, depending on the specifics of their intended use. In preferred embodiments, the tissue specific marker contains at least a targeting element and an indicator element. One example of the tissue specific marker is illustrated in FIG. 8A which includes a targeting element (e.g., aptamer or affimer component) 415 and an indicator element 410. Generally, the targeting element is linked to the indicator element directly or via a linker molecule.

a. Targeting Elements of Tissue Specific Markers

Tissue specific markers provided herein generally include a targeting element such as an aptamer or affimer. Such aptamer or affimer component typically binds to target tissue with relatively high affinity, particularly when compared to other tissues such as neighboring tissue or other undesirable tissue. In general, the affimer or aptamer selectively binds to the target tissue over at least one other tissue. In some cases, the aptamer or affimer of a tissue specific marker may have affinity for more than one target. For example, the aptamer or affimer may have relatively high affinity for target tissue as well as relatively high affinity for a different tissue. In some cases, the aptamer binds to the non-malignant target tissue with an affinity or a binding affinity (K_{d} or dissociation constant) within a range of 1 pM to 1 mM.

In preferred embodiments, the aptamer or affimer component of the tissue specific markers provided herein has high affinity for parathyroid tissue, particularly healthy or normal parathyroid tissue. In some cases, the targeting elements have high affinity for one or more abnormal (e.g., adenoma, hyperplasia or malignant tumor) parathyroid tissue. In some cases, an aptamer or affimer component may specifically bind to one of the following tissues: parathyroid, nerve, reproductive organ, cervix, ovary, endometrium, breast, colon, fallopian tube, gall bladder, jejunum, liver, lung, esophagus, pancreas, pituitary, placenta, prostate, skin, spinal, stomach, testes, tonsil, ureter, kidney, muscle, spleen, bladder, cerebellum, brain cortex, or other tissue. In some preferred embodiments, the bound tissue is parathyroid tissue, nerve tissue and/or reproductive tissue (e.g., tissue derived from cervix, ovary, endometrium or other female reproductive organ). In some cases, the aptamer or affimer component may specifically bind to tissue that originated from an endodermal, ectodermal, or mesodermal germ layer. Preferably, the targeting element binds to organ tissue, particularly a solid organ or gland.

A targeting element provided herein generally can selectively bind to a specific tissue over undesirable tissue. Such undesirable tissue may be tissue neighboring or adjacent to target tissue. In some further preferred embodiments, the targeting element selectively binds to parathyroid tissue (healthy, abnormal, or both) with higher affinity than to thyroid tissue. In some cases, to differentiate neighboring tissue from the targeted tissue, the tissue specific markers bind with relatively high affinity to the target tissue with minimal off-target binding. In some preferred embodiments, the aptamer selectively binds to a parathyroid gland or a parathyroid adenoma with an affinity at least 2-fold, at least 5-fold, at least 10-fold or at least 20-fold higher than an affinity of the aptamer to a thyroid gland.

As used herein, the term “aptamer” generally refers to oligonucleotides or peptides that specifically or selectively bind to a target (e.g., target tissue, target molecule, target cell). Generally, oligonucleotide aptamers may contain DNA, RNA, and/or modified nucleic acids in any combination. For example, in some cases, oligonucleotide aptamers may be entirely or partially made up of DNA or RNA; while in other cases they may comprise both DNA and RNA. Theoretically, the oligonucleotide aptamers provided herein likely bind to target molecules or tissue through non-Watson/Crick interactions. In some cases, the aptamers and/or affimers provided herein do not occur naturally. For example, the aptamers and/or affimers may comprise a sequence that does not occur in nature or may have one or more modifications that do not occur in nature. Affimers can be small peptides or proteins, generally with a molecular weight less than 12 kDa. Aptamers or affimers can have the capacity to recognize specific epitopes or antigens, and with binding affinities that can be close to those of antibodies (e.g., in the low nanomolar to picomolar range); however, the terms “aptamer” or “affimer,” as used herein, do not encompass antibodies, immunoglobulins, Fab regions of antibodies, or Fc regions of antibodies. Aptamers can have the same specificity advantage of antibodies, but can be smaller, can be chemically synthesized or chemically modified, and have the advantage of being free from cell culture contaminants.

The aptamers and affimers provided herein may be non-immunogenic or demonstrate limited ability to provoke an immune response. The aptamers and affimers provided herein may, in some cases, be able to bind to extracellular targets, a characteristic that is important for designing tissue specific markers. Once designed and selected, aptamers are generally stable, easy to handle and inexpensive to manufacture. Their chemical composition also allows the integration of a broad range of indicator elements.

Aptamers provided herein can be any length or size. In some cases, an aptamer provided herein is in the range of 80-100 nucleotides in length. In some cases, the aptamer is up to 100 nucleotides in length.

Aptamers can be selected and designed depending on their desired function. The SELEX process, when applied to aptamers, can comprise multiple steps. Steps include synthesis of a very large oligonucleotide library consisting of unique randomly generated sequences. Sequences can be of fixed length, and can comprise 5′ and 3′ ends that are shared amongst portions of the library or the entire library. The 5′ and 3′ ends can be configured to serve as primer recognition sequences for amplifying sequences in the library. The sequences in the library can be exposed to a target ligand (e.g., a protein, small organic molecule, tissue, etc.), and those sequences that do not bind to the target ligand can be removed (e.g., by washing, through affinity chromatography or other means). Bound sequences can be eluted and amplified by PCR to prepare for subsequent rounds of selection. In subsequent rounds of selection, the stringency of the binding conditions can be increased to identify and select for the aptamer sequences with a high affinity or selectivity for target ligand. There are many variants of the SELEX method that have been used for aptamer selection, including counter selection, in which aptamers are selected for their property of binding to a different target, and are then discarded.

The aptamer component of a tissue specific marker provided herein may contain a sequence identical or similar to any one of SEQ ID NOs: 1-200. In some cases, the aptamer comprises a sequence with at least 70% sequence identity to any one of SEQ ID NOs: 1-200, at least 80% sequence identity to any one of SEQ ID NOs: 1-200, at least 85% sequence identity to any one of SEQ ID NOs: 1-200, at least 90% sequence identity to any one of SEQ ID NOs: 1-200, or at least 95% sequence identity to any one of SEQ ID NOs: 1-200. The aptamer component of a tissue specific marker provided herein may contain a sequence identical or similar to any one of SEQ ID NO:1, SEQ ID NO:2, SEQ ID NO:3, SEQ ID NO:4, SEQ ID NO:5, SEQ ID NO:101, SEQ ID NO:102, SEQ ID NO:103, SEQ ID NO:104, or SEQ ID NO:105. In some cases, the aptamer comprises a sequence with at least 70% sequence identity to any one of SEQ ID NO:1, SEQ ID NO:2, SEQ ID NO:3, SEQ ID NO:4, SEQ ID NO:5, SEQ ID NO:101, SEQ ID NO:102, SEQ ID NO:103, SEQ ID NO:104, or SEQ ID NO:105, at least 80% sequence identity to any one of SEQ ID NO:1, SEQ ID NO:2, SEQ ID NO:3, SEQ ID NO:4, SEQ ID NO:5, SEQ ID NO:101, SEQ ID NO:102, SEQ ID NO:103, SEQ ID NO:104, or SEQ ID NO:105, at least 85% sequence identity to any one of SEQ ID NO:1, SEQ ID NO:2, SEQ ID NO:3, SEQ ID NO:4, SEQ ID NO:5, SEQ ID NO:101, SEQ ID NO:102, SEQ ID NO:103, SEQ ID NO:104, or SEQ ID NO:105, at least 90% sequence identity to any one of SEQ ID NO:1, SEQ ID NO:2, SEQ ID NO:3, SEQ ID NO:4, SEQ ID NO:5, SEQ ID NO:101, SEQ ID NO:102, SEQ ID NO:103, SEQ ID NO:104, or SEQ ID NO:105, or at least 95% sequence identity to any one of SEQ ID NO:1, SEQ ID NO:2, SEQ ID NO:3, SEQ ID NO:4, SEQ ID NO:5, SEQ ID NO:101, SEQ ID NO:102, SEQ ID NO:103, SEQ ID NO:104, or SEQ ID NO:105. In some cases, the aptamer comprises a sequence of SEQ ID NO:3, SEQ ID NO:4, SEQ ID NO:5, SEQ ID NO:103, SEQ ID NO:104 or SEQ ID NO:105. In some cases, the aptamer comprises a sequence with at least 70% sequence identity to SEQ ID NO:3, SEQ ID NO:4, SEQ ID NO:5, SEQ ID NO:103, SEQ ID NO:104 or SEQ ID NO:105. In some cases, the aptamer comprises a sequence with at least 80% sequence identity to SEQ ID NO:3, SEQ ID NO:4, SEQ ID NO:5, SEQ ID NO:103, SEQ ID NO:104 or SEQ ID NO:105. In some cases, the aptamer comprises a sequence with at least 90% sequence identity to SEQ ID NO:3, SEQ ID NO:4, SEQ ID NO:5, SEQ ID NO:103, SEQ ID NO:104 or SEQ ID NO:105. In some cases, the aptamer comprises a sequence with at least 95% sequence identity to SEQ ID NO:3, SEQ ID NO:4, SEQ ID NO:5, SEQ ID NO:103, SEQ ID NO:104 or SEQ ID NO:105.

In some cases, the aptamer may comprise a sequence that includes a GATACTG motif. In some cases, the aptamer may comprise a GATACTG motif with 1, 2, 3, 4 or more nucleotide substitutions, insertions, transpositions or deletions. For example, the aptamer may comprise a sequence that includes GANACTG motif, wherein N is dG, dC, dT, or dA. In some cases, an aptamer comprises an inverted T at a 3′ end of the aptamer.

b. Indicator Elements of Tissue Specific Markers

The aptamer or affimer may localize the marker to the targeted tissue, but for such targeting element to be detected it is preferably coupled to one or more indicator elements 410 that produces a signal that can be detected by the surgeon or that can otherwise be detected by a probe or detector instrument. Generally, the indicator elements can be detected by the surgeon either through the skin for preoperative applications or intraoperatively, within the surgical site. The indicator element may be any type of detectable label including a fluorophore, a dye, a nanodiamond, a quantum dot, gold nanoparticle, nanorod (e.g., gold nanorod), magnetic bead, iron oxide or gold particles, aggregation-induced emission dot, or a nanocrystal. In some cases, the indicator element can emit visible light or near infrared when exposed to or excited by energy. The markers may be easily detected directly, through paramagnetism, optoacoustics or optically either within the visible light spectrum or outside of the visible region, for example in the near-infrared, with the use of a probe or a detector instrument (e.g., a complementary metal-oxide-semiconductor (CMOS) camera, a charge coupled display (CCD) camera).

In preoperative applications of the tissue specific marker, a signal from an indicator element may be detected above the skin in order to identify the location of a tissue specific marker that has already been introduced into a patient. Detection of a tissue specific marker in such manner may assist the surgeon in deciding, for example, where to make an incision or if the surgical procedure needs to occur. In some cases, an indicator element may be detected after the surgery has begun to distinguish the targeted tissue from neighboring tissue either when removing the targeted tissue or when removing tissue adjacent to the targeted tissue.

Optionally, the tissue specific marker as seen in FIG. 8C may contain a second indicator element 425 that can be coupled as well to the aptamer or affimer or any other part of the tissue specific marker including the first indicator element 410. Optionally, the marker may be PEGylated as shown by polyethylene glycol moiety 420. In other examples, the marker may not be PEGylated. The tissue specific marker as seen in FIG. 8C may be used to identify the target tissue preoperatively and/or during a surgical procedure. For example, the first indicator element may be used during a surgical procedure to locate the target tissue, or the undesirable tissue, as described herein and/or the second indicator element may be used preoperatively. In cases where the first indicate element is used during a surgical procedure, the first indicator element may be any indicator element and need not be an indicator element that is detectable through the skin. The second indicator element may allow the tissue specific marker, and by extension targeted tissue, to be located preoperatively, before the surgeon makes an incision. A signal emitted by the second indicator element may often be detectable through the skin. As such, the second indicator element may contain a nanodiamond, iron oxide particles, etc. in some instances. As mentioned herein, preoperative identification of the tissue specific marker may, for example, enable the surgeon to make a better informed decision when deciding if surgery is necessary or when selecting an incision site. In some particular examples, the tissue specific marker contains: (a) a parathyroid-specific aptamer or affimer; (b) a first indicator element that is a fluorophore; and (c) a second indicator element that is detectable through the skin, such as a nanodiamond or a paramagnetic bead.

The first and the second indicator elements generally are distinguishable. One or both of the indicator elements may be optoacoustic or magnetic. One or both indicator elements may comprise a bead, fluorophore, nanoparticle such as a gold nanoparticle, nanorod such as a gold nanorod, quantum dot, nanocrystal or combination thereof. In some cases, the first and second indicator elements are the same type of indicator element such as a fluorescent protein and are distinguishable by color or emission wavelength. In some cases, the first and second indicator elements are different types of indicator elements (e.g., nanodiamond and an Indocyanine Green (ICG) fluorophore). In some cases the indicator element may comprise a pH sensitive molecule.

Any number of indicator elements may be coupled to the aptamer or affimer to allow detection by any number of means including visualization by the surgeon, or by use of a probe or other device that can detect the indicator element. For example, an aptamer can be synthesized with an amine group at the 5′ end, to which an indicator element (e.g., fluorophore) can be conjugated. Fluorophores can be included that emit in the visible or in the near-infrared range. In some cases, the first indicator element is a fluorophore and the second indicator element is a different fluorophore. The indicator elements may be attached to the same aptamer or affimer or to different aptamers or affimers. In some cases, at least 1, at least 2, at least 3, at least 4, or at least 5 indicator elements attached to different aptamers or affimers are used. In some cases, at least 1, at least 2, at least 3, at least 4, or at least 5 indicator elements attached to the same aptamer or affimer are used.

Aptamers or affimers provided herein can be conjugated with indicator elements including near-infrared dyes or cyanine dyes such as Indocyanine Green (ICG). A non-limiting list of near-infrared indicator elements that may be used in the present disclosure includes: ICG, IRDye800CW, non-sulfonated cyanine dyes, conjugated copolymers, quantum dots, aggregation-induced emission dots, metal nanoclusters, single-walled carbon nanotubes, IR-PEG nanoparticles, and/or infrared fluorescent proteins.

The indicator element can be directly conjugated to various portions of the aptamer, or may be attached using linkers or other means of covalently attaching the dye. For example, an aptamer can be synthesized with an amine group at the 5′ end, to which an indicator element (e.g., fluorophore or dye) can be conjugated.

c. Modifications to Tissue Specific Markers

Once administered to the patient, the tissue specific marker may need to remain intact for a long enough period of time so that it can localize and bind to target tissue and present a signal before being degraded or cleared (excreted) from the body. To prevent the tissue specific marker from being degraded before or while it is binding specifically to the targeted tissue, the tissue specific marker may be synthesized using modified nucleotides or nucleosides, which can reduce or prevent degradation. Additionally as illustrated in FIG. 8B, the tissue specific marker may optionally be chemically modified. For example, the tissue specific marker may be PEGylated, or modified to contain at least one bulky polyethylene glycol moiety 420, to reduce clearance of the tissue specific marker via the bloodstream.

The aptamer or affimer can contain modifications designed to increase stability and prevent degradation. In some cases, the tissue specific marker can be modified or chemically altered to be more resistant to DNases in the blood. In some cases, a tissue specific marker provided herein can have a clearance time or half-life of about or at least about 2, 3, 4, 5, 10 or 20 minutes.

Examples of aptamer modifications can include, but are not limited to, PEGylation, chemical modification of the nucleic acid backbone using nucleotide analogs, the addition of an inverted Tat the 3′ end of the aptamer, incorporation of locked nucleic acid (LNA molecules) that contain methylene bridges, and conjugation to other functional groups such as PEG and cholesterol. Aptamer stability can be tested in vitro. Experiments can demonstrate that introducing modifications to the aptamers (e.g., addition of inverted T at the 3′ end of the aptamer, or PEGylation of the aptamer) can reduce aptamer degradation and allow the aptamers to demonstrate better performance at a range of temperatures and conditions. For example, aptamer stability can be tested by incubating an aptamer with blood and removing aliquots over time. The DNA can be isolated, and a qPCR reaction can be run to quantify the amount of aptamer that remains intact as a function of incubation time.

### Surgical Use

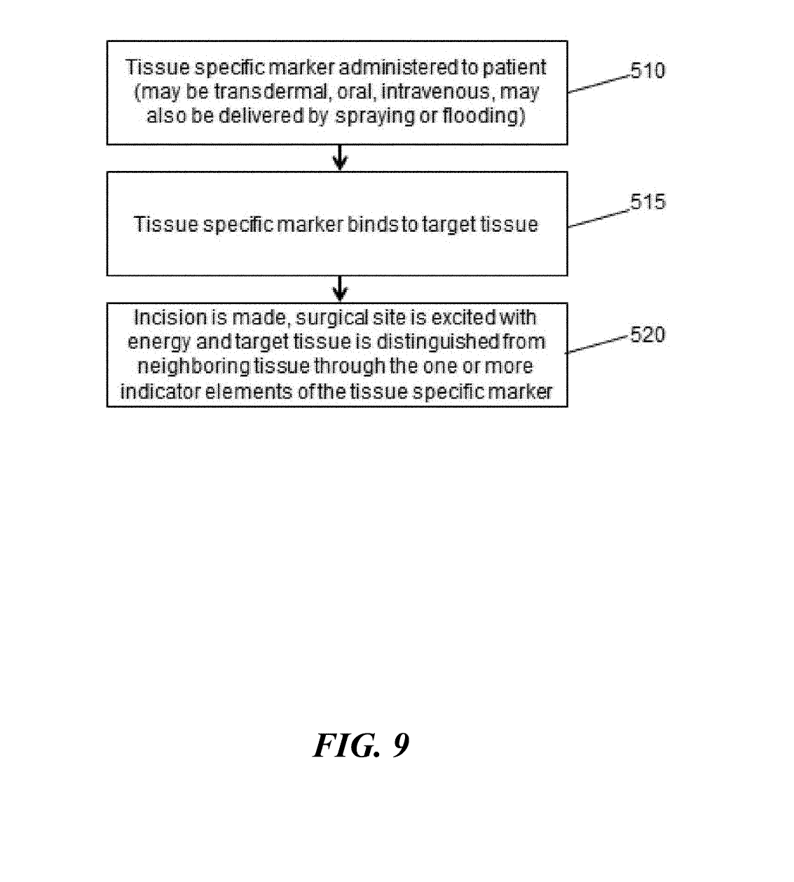

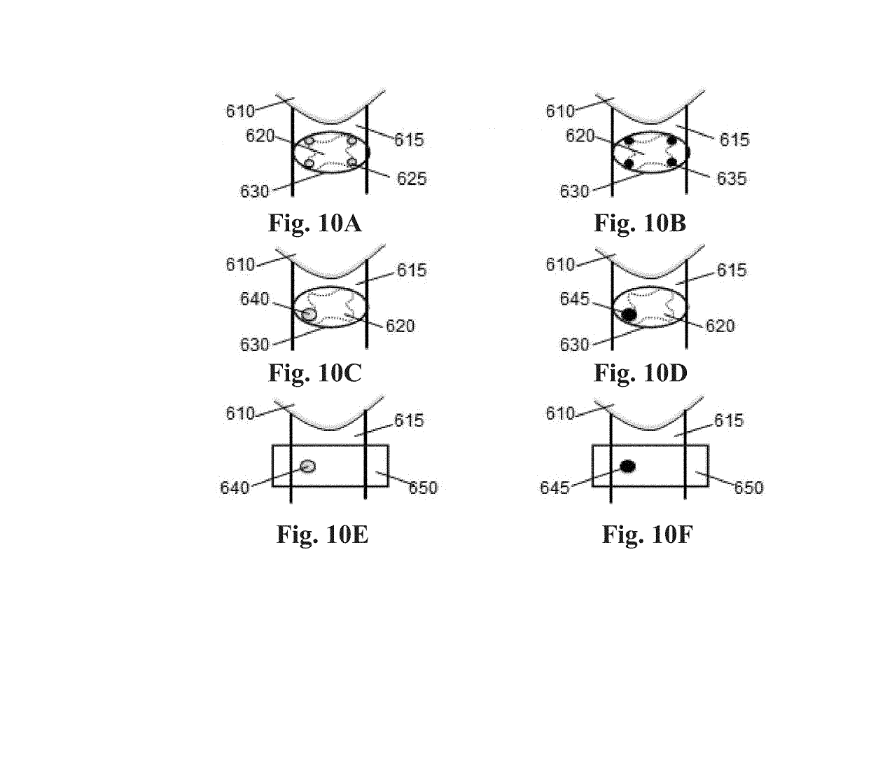

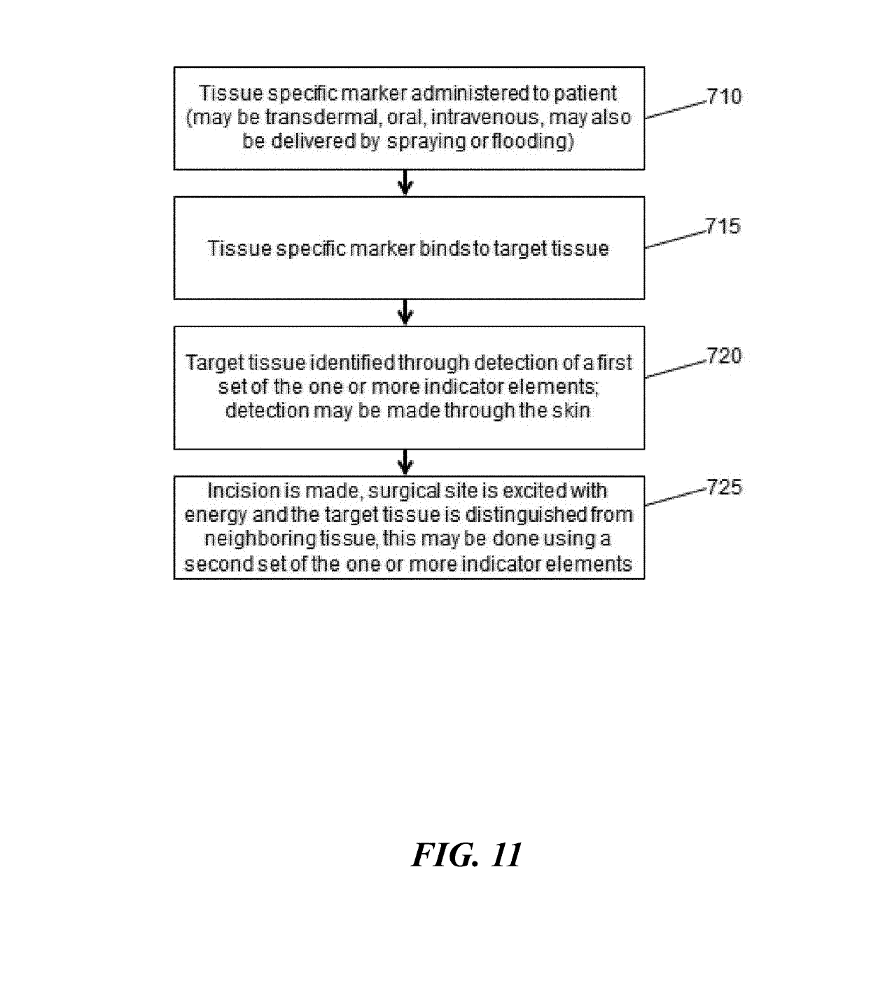

This disclosure includes methods of using tissue specific markers for surgical purposes. Tissue specific markers can be used intraoperatively and/or preoperatively. An example of an intraoperative method of using the tissue specific marker is shown in FIG. 9. First the marker is administered to the patient 510; this administration may be transdermal, oral, intravenous, through a spray, or by flooding. Administration may include a washing step, or multiple washing steps. Once administered, the aptamer or affimer component of the tissue specific marker binds with high specificity to the targeted tissue 515. In the case where the design of the maker has only one indicator, an incision is made to uncover the surgical site. The surgical site is then exposed and can be excited by energy, for example near-infrared, and the indicator element will produce a detectable spectral signal 520. In some cases, the surgical site is not excited. Since the tissue specific marker containing the indicator element is uniquely localized to the target tissue and not neighboring tissue, only the target tissue will produce the spectral signal, allowing the surgeon to clearly distinguish one tissue from the other. In the intraoperative use of the tissue specific marker in thyroidectomy or parathyroidectomy procedures (FIG. 10) the surgeon may make an incision 630 in the frontal middle of the neck 615, chin 610 of patient is illustrated in FIGS. 10A-F for perspective. The tissue specific marker may be used to mark the parathyroid tissue 625 as seen in FIG. 10A, and upon excitation shown in FIG. 10B the tissue specific marker specifically located in the parathyroid tissue may produce a signal 635 that the surgeon can detect and use to guide the procedure enabling removal of the thyroid tissue 620 while leaving the signal producing parathyroid tissue 635 intact or can enable the removal of the affected parathyroid gland or glands with minimal disruption to neighboring thyroid tissue 620. Optionally, the tissue specific marker can also be used intraoperatively, as shown in FIG. 10C, to specifically label parathyroid tissue including adenoma tissue 640. Here the aptamer or affimer component localizes to adenoma tissue 640 which upon excitation, shown in FIG. 10D, produces a signal 645 that the surgeon can use to easily identify and remove the adenoma tissue from neighboring tissue. The aptamer can either be specific for parathyroid adenoma, and not normal parathyroid, or it can be designed to recognize both.

Optionally, the doctor or surgeon may elect to administer two or more markers simultaneously, for example one targeted specifically to abnormal tissue, for example an adenoma 640, and another one for example healthy thyroid tissue 620, and the doctor or surgeon may use the presence or absence of a signal from the targeted tissue to extract an affected parathyroid while avoiding the healthy parathyroid glands as well as the thyroid tissue. By using two or more tissue specific markers the surgeon can build a surgical roadmap, where multiple critical tissues are highlighted. For example, differentiating sentinel lymph nodes from normal nodes during removal of cancerous breast tissue. Optionally, the tissue specific markers may also be used preoperatively as a diagnostic to guide a doctor or surgeon in deciding whether a surgical procedure is necessary, or it may also be optionally used to guide the selection of an incision site once a diagnosis has already been made. In a diagnostic or preoperative application, the tissue specific marker may be localized to the target tissue, for example the adenoma 640 shown in FIG. 10E, and using a detector the doctor or surgeon may detect a signal 645 from the tissue specific marker as illustrated in FIG. 10F. Optionally, the doctor or surgeon may detect the signal from the tissue specific marker, and may further use the signal to guide selection of an incision site before making an incision. This tissue specific marker can be used pre- and intraoperatively, until an optional second marker's signal (e.g., fluorescence) is visible.

FIG. 11 illustrates options that may be used for combining preoperative and intraoperative use of the tissue specific marker. The tissue specific marker can be administered to the patient 710, and the aptamer or affimer may then bind to the targeted tissue 715. The doctor or surgeon may use a detector to identify the location of the target tissue through the one or more indicator elements of the tissue specific marker 720. The doctor or surgeon may administer the tissue specific marker to a targeted tissue, and the indicator element of the tissue specific marker may be detected by a probe or detector 650 through the patient's skin prior to surgery as shown in FIG. 10F, or it may be visually observed by the doctor or surgeon.

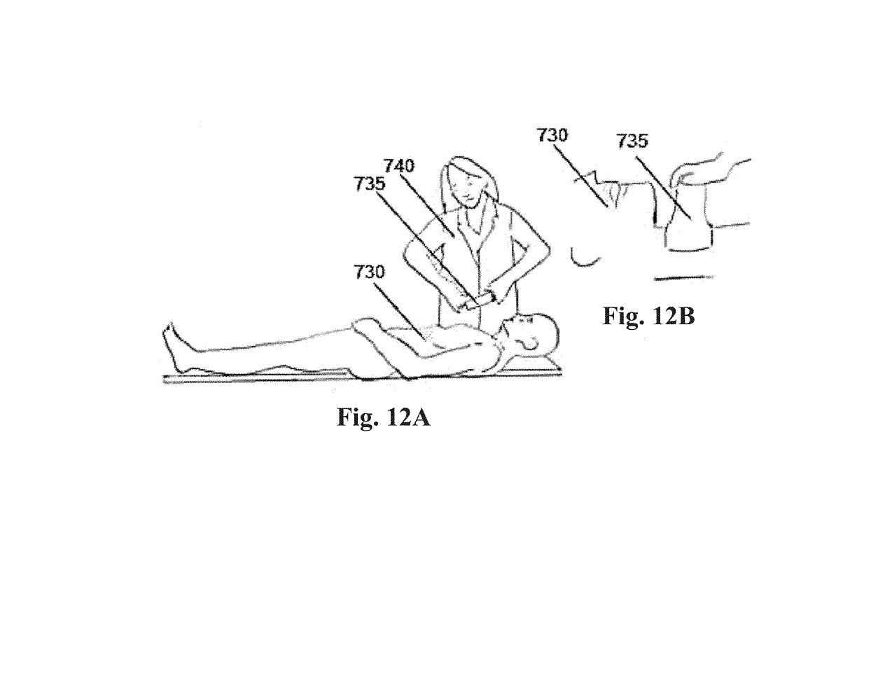

FIGS. 12A-12B illustrate the use of a probe to detect the tissue specific marker for diagnostic or preoperative purposes. After administering the tissue specific marker, the doctor 740 or surgeon may use a probe 735 to detect the presence or absence of a signal from the patient 730 to assist in making a diagnosis. Optionally the doctor 740 or surgeon may use a probe 735 to detect the tissue specific marker for making a better informed decision about the location of the target tissue allowing precise selection of an incision site using any of the techniques described herein. Further, the surgeon may expose or excite the surgical site with energy and use the tissue specific marker to distinguish the targeted tissue 725 from neighboring tissue.

### Systems

This disclosure includes systems for differentiating target tissue from adjacent tissue. In some cases, the system comprises a tissue specific marker described herein and a probe for exciting one or more indicator elements with energy. In some cases, the system comprises a probe for detecting the signal from the one or more indicator elements. The excitation probe may be attached to or separate from the detector probe.

In some cases, the system comprises: a tissue-specific marker described herein; a second tissue specific marker, comprising a second aptamer or a second affimer configured to bind to a second pre-selected target tissue. The second tissue specific marker may comprise one or more second indicator elements coupled with the second aptamer or the second affimer, wherein the second indicator element produces a signal when exposed to or excited by energy thereby allowing identification of the second target tissue. Such system may comprise a first probe for exciting the first and/or second tissue specific marker with energy. Such system may comprise a probe for detecting the signal from the one or more indicator elements. In some cases, such system may comprise a second probe for exciting the second tissue specific marker with energy. Such system may also contain a second probe for detecting the signal from the one or more second indicator elements.

In some cases, a system described herein further comprises an illumination source. In some cases, a system described herein further comprises a camera. For example, the system may comprise a complementary metal-oxide-semiconductor (CMOS) camera or a charge coupled display (CCD) camera.

### EXAMPLES

### Example 1: Selection of Aptamers that Selectively Bind Parathyroid Tissue

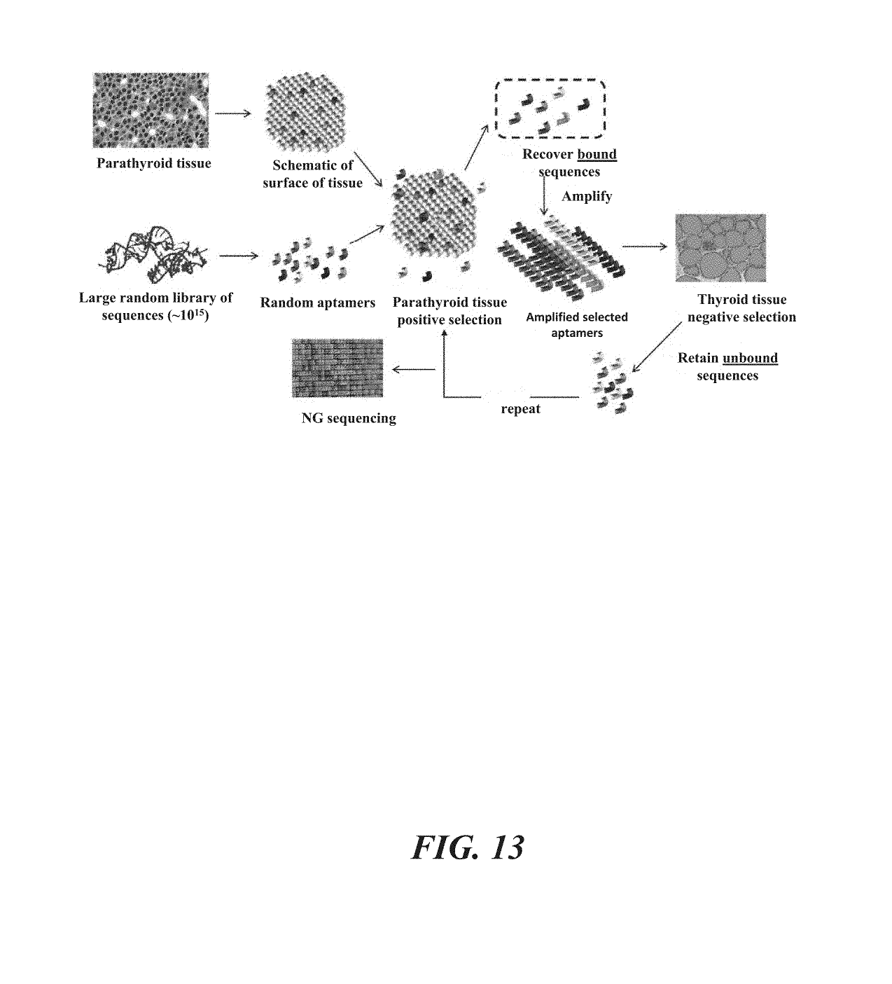

FIG. 13 illustrates the design of the SELEX process that resulted in the isolation of aptamers that bound parathyroid with high affinity and did not bind normal thyroid tissue. Briefly, single stranded DNA aptamers that bind specifically or with high affinity to parathyroid tissue can be selected using SELEX procedures known in the art. In this particular instance, the selection library included oligonucleotide sequences 84 nucleotides in length, of which 40 nucleotides were randomized, the first 23 nucleotides (5′-TAGGGAAGAGAAGGACATATGAT-3′) were conserved and served as the forward primer recognition sequence, and the last 21 nucleotides (5′-TTGACTAGTACATGACCACTT-3′) were conserved and served as the reverse primer recognition sequence. For positive selection, the library of aptamers was allowed to bind to normal human parathyroid tissue on a slide at room temperature. After 15 minutes, the slide was washed and all aptamers that were not tightly bound were eluted and discarded; the resulting library was amplified by PCR. The selection strategy also included alternating negative selection with normal human thyroid tissue. For negative selection, the library of sequences that was selected by specifically binding to parathyroid and amplified was allowed to bind to normal thyroid tissue slides. After incubation, the bound aptamers were discarded, and the ones that did not bind thyroid were selected and amplified with PCR. In this fashion, normal human parathyroid and thyroid tissue slides were used alternatively to select aptamers that bind parathyroid with much higher affinity than to thyroid. Negative selection allowed the identification and removal of those aptamers that even though they may exhibit a high affinity for parathyroid, also bind tightly to thyroid, and therefore discarded.

The 100 sequences with the highest affinity to parathyroid and much lower affinity to thyroid were analyzed by next generation sequencing and are listed in Table 1 with the primer recognition sequences and in Table 2 without the primer recognition sequences. Once identified through this process, the aptamers can be easily and inexpensively generated through PCR or de novo DNA synthesis.

TABLE 1
Full-Length Sequences Including Primer Recognition
Sequences in order of Sequence Abundance

| SEQ ID NO | Sequence |
|---|---|
| SEQ ID NO: 1 | TAGGGAAGAGAAGGACATATGATCGTTTGGGCCTTATGTACGGCGTAAAT |
| | TTCTCCTGCAGAATTGACTAGTACATGACCACTT |
| SEQ ID NO: 2 | TAGGGAAGAGAAGGACATATGATCCACTTATCTAGCGTAGATAAGGCGTT |
| | TAAAAGGTCTAACTTGACTAGTACATGACCACTT |
| SEQ ID NO: 3 | TAGGGAAGAGAAGGACATATGATTTCGGTCTAGCACACTCAACGAGATAC |
| | TGGGGTTAAACGTTTGACTAGTACATGACCACTT |
| SEQ ID NO: 4 | TAGGGAAGAGAAGGACATATGATCCCGATACTGAAAATGGAGGCCCGCAA |
| | GTATTATTTACAATTGACTAGTACATGACCACTT |
| SEQ ID NO: 5 | TAGGGAAGAGAAGGACATATGATCACTTCATGTAAGACTAAAAGATGGAG |
| | CGTGAAGGATGCATTGACTAGTACATGACCACTT |
| SEQ ID NO: 6 | TAGGGAAGAGAAGGACATATGATGTGGGTTAACTAATGAGGCTTAACGAG |
| | GCGTCAACGTTTTTTGACTAGTACATGACCACTT |
| SEQ ID NO: 7 | TAGGGAAGAGAAGGACATATGATCGACACTGTTTGTAAGTCTTCCCTGAT |
| | TACTTATTTCATCTTGACTAGTACATGACCACTT |
| SEQ ID NO: 8 | TAGGGAAGAGAAGGACATATGATTTCCAGACGTTATTAGCCCGATCTCCT |
| | GTGTACGATCCAGTTGACTAGTACATGACCACTT |
| SEQ ID NO: 9 | TAGGGAAGAGAAGGACATATGATGTATATGTACCAACCGAGTGATTCGGC |
| | CTATCAAAGCGTCTTGACTAGTACATGACCACTT |
| SEQ ID NO: 10 | TAGGGAAGAGAAGGACATATGATCGTTTGGGCCCTATGTACGGCGTAAAT |
| | TTCTCCTGCAGAATTGACTAGTACATGACCACTT |
| SEQ ID NO: 11 | TAGGGAAGAGAAGGACATATGATAAAAAACCGGGGTTCTTAATTTTCATT |
| | GTTCGTCGTACTTTTGACTAGTACATGACCACTT |
| SEQ ID NO: 12 | TAGGGAAGAGAAGGACATATGATTACAAGTAAAACTGACCCCTCCATTTG |
| | TGTGTTTATTCGCTTGACTAGTACATGACCACTT |
| SEQ ID NO: 13 | TAGGGAAGAGAAGGACATATGATACCAATCATACTAAGCCTACCCGAACC |
| | AGCGGTGGAATGGTTGACTAGTACATGACCACTT |
| SEQ ID NO: 14 | TAGGGAAGAGAAGGACATATGATCGTTTGGGCCTTACGTACGGCGTAAAT |
| | TTCTCCTGCAGAATTGACTAGTACATGACCACTT |
| SEQ ID NO: 15 | TAGGGAAGAGAAGGACATATGATCGTTTGGGCCTTATGTACGGCGTAAAT |
| | TTCTCCTGCAGAGTTGACTAGTACATGACCACTT |
| SEQ ID NO: 16 | TAGGGAAGAGAAGGACATATGATTTAGTACGTATTGATATGATCTGAACT |
| | ATGTGAGAATGAGTTGACTAGTACATGACCACTT |
| SEQ ID NO: 17 | TAGGGAAGAGAAGGACATATGATCGTTTGGGCCTTATGTACGGCGTAAAC |
| | TTCTCCTGCAGAATTGACTAGTACATGACCACTT |
| SEQ ID NO: 18 | TAGGGAAGAGAAGGACATATGATCCGTCTATTGCCGAGGATGGGTAATAG |
| | TACCGTGCGCACTTTGACTAGTACATGACCACTT |
| SEQ ID NO: 19 | TAGGGAAGAGAAGGACATATGATCGTTTGGGCCTTATGTACAGCGTAAAT |
| | TTCTCCTGCAGAATTGACTAGTACATGACCACTT |
| SEQ ID NO: 20 | TAGGGAAGAGAAGGACATATGATTGTTTGGGCCTTATGTACGGCGTAAAT |
| | TTCTCCTGCAGAATTGACTAGTACATGACCACTT |
| SEQ ID NO: 21 | TAGGGAAGAGAAGGACATATGATCGTGAGGGAGACCCTACGAGAGAGAAA |
| | AGAAAAGGAAAAGTTGACTAGTACATGACCACTT |
| SEQ ID NO: 22 | TAGGGAAGAGAAGGACATATGATCGTTTGGGCCTTATGTACGGCGTAAAT |
| | TTCTCCTACAGAATTGACTAGTACATGACCACTT |
| SEQ ID NO: 23 | TAGGGAAGAGAAGGACATATGATATAGAATGAGGAGGTCACCAATGGACA |
| | CTAATCGACCGTATTGACTAGTACATGACCACTT |
| SEQ ID NO: 24 | TAGGGAAGAGAAGGACATATGATGGAGGAAAAGAGACAACAAAGAACGCC |
| | GCGCACAAGGCACTTGACTAGTACATGACCACTT |
| SEQ ID NO: 25 | TAGGGAAGAGAAGGACATATGATCGTTGATTTACGGTCGTAAGCGGTACG |
| | GTTTCATCGTCAGTTGACTAGTACATGACCACTT |
| SEQ ID NO: 26 | TAGGGAAGAGAAGGACATATGATCGTTTGGGCCTTATGTACGGTGTAAAT |
| | TTCTCCTGCAGAATTGACTAGTACATGACCACTT |
| SEQ ID NO: 27 | TAGGGAAGAGAAGGACATATGATCGTTTGGGCCTTATGTACGGCGTAAAT |
| | TTCTCCCGCAGAATTGACTAGTACATGACCACTT |
| SEQ ID NO: 28 | TAGGGAAGAGAAGGACATATGATCGTTTGGGCCTTATGTACGGCGTAAAT |
| | TTCCCCTGCAGAATTGACTAGTACATGACCACTT |
| SEQ ID NO: 29 | TAGGGAAGAGAAGGACATATGATCGTTTGGGCCTTATGTACGGCGTAAAT |
| | TTCTCCTGCAGGATTGACTAGTACATGACCACTT |
| SEQ ID NO: 30 | TAGGGAAGAGAAGGACATATGATCGAGGGTCCTGTTGACTACGTCTTTGA |
| | ACTCATTGGTCACTTGACTAGTACATGACCACTT |
| SEQ ID NO: 31 | TAGGGAAGAGAAGGACATATGATCGTTTAGGCCTTATGTACGGCGTAAAT |
| | TTCTCCTGCAGAATTGACTAGTACATGACCACTT |
| SEQ ID NO: 32 | TAGGGAAGAGAAGGACATATGATCGTTTGGGCCTCATGTACGGCGTAAAT |
| | TTCTCCTGCAGAATTGACTAGTACATGACCACTT |
| SEQ ID NO: 33 | TAGGGAAGAGAAGGACATATGATGGTCTAGAACAGAGATAACCAACATTG |
| | TCCCGAAAAGCCCTTGACTAGTACATGACCACTT |
| SEQ ID NO: 34 | TAGGGAAGAGAAGGACATATGATTGCTGATCCCAGCAAACGGTATGACGC |
| | AACAGAGGTATCATTGACTAGTACATGACCACTT |
| SEQ ID NO: 35 | TAGGGAAGAGAAGGACATATGATGAGACAAATGATGTCCATGCATGCCGC |
| | CAAACAACCGAGATTGACTAGTACATGACCACTT |
| SEQ ID NO: 36 | TAGGGAAGAGAAGGACATATGATCCTTCAGAAAGGAACATATGCCGTTAA |
| | GACCAGAACTTCGTTGACTAGTACATGACCACTT |
| SEQ ID NO: 37 | TAGGGAAGAGAAGGACATATGATCGTTTGGGCCTTATGCACGGCGTAAAT |
| | TTCTCCTGCAGAATTGACTAGTACATGACCACTT |
| SEQ ID NO: 38 | TAGGGAAGAGAAGGACATATGATCACGGAAACGTGTAGAATTACACGTTA |
| | ACGAAGTGAGGAGTTGACTAGTACATGACCACTT |
| SEQ ID NO: 39 | TAGGGAAGAGAAGGACATATGATCGTTTGGGCCTTGTGTACGGCGTAAAT |
| | TTCTCCTGCAGAATTGACTAGTACATGACCACTT |
| SEQ ID NO: 40 | TAGGGAAGAGAAGGACATATGATCAAACGTCGAATACGGATTGTCAAACG |
| | AACAACACCGTATTTGACTAGTACATGACCACTT |
| SEQ ID NO: 41 | TAGGGAAGAGAAGGACATATGATATGGCCATCTGCCTAGTCATAGATTGG |
| | GAATCTGAACCGATTGACTAGTACATGACCACTT |
| SEQ ID NO: 42 | TAGGGAAGAGAAGGACATATGATGGCAGTCAGAAAGCTCTCGAGAATGTG |
| | GACCCGAGAGCAGTTGACTAGTACATGACCACTT |
| SEQ ID NO: 43 | TAGGGAAGAGAAGGACATATGATGCCGTGTCGAGGAACATCCTAGACAAG |
| | GTGAAAAGTCCCATTGACTAGTACATGACCACTT |
| SEQ ID NO: 44 | TAGGGAAGAGAAGGACATATGATCAAGTAGCATAGTGGACGAACGAGCGG |
| | AACAAATGCGTAATTGACTAGTACATGACCACTT |
| SEQ ID NO: 45 | TAGGGAAGAGAAGGACATATGATTGCGATGCGAGTAGAAAAGCGTATGCT |
| | ACAGGAACGTCCATTGACTAGTACATGACCACTT |
| SEQ ID NO: 46 | TAGGGAAGAGAAGGACATATGATTTTATCAGAGACGCGCCCTTAGCAAAC |
| | GTGTTCTTCCGAATTGACTAGTACATGACCACTT |
| SEQ ID NO: 47 | TAGGGAAGAGAAGGACATATGATTATATGGAATCTCCAATGTGGCAACAG |
| | GACACATAAAGAATTGACTAGTACATGACCACTT |
| SEQ ID NO: 48 | TAGGGAAGAGAAGGACATATGATGCCGGATCAACCCAAGGAGTTGATTAG |
| | CATCATTTTACGATTGACTAGTACATGACCACTT |
| SEQ ID NO: 49 | TAGGGAAGAGAAGGACATATGATGTAGGGGTCCACGAAGTGCTAAGAAGG |
| | CACACATTTTGCGTTGACTAGTACATGACCACTT |
| SEQ ID NO: 50 | TAGGGAAGAGAAGGACATATGATAACAGCGCGCGCCCTCAACGATAGACT |
| | ATAAGTCCAAAGGTTGACTAGTACATGACCACTT |
| SEQ ID NO: 51 | TAGGGAAGAGAAGGACATATGATGTACGCGTCAGAGTGTCGTGAACGACA |
| | AACGACTGATACCTTGACTAGTACATGACCACTT |
| SEQ ID NO: 52 | TAGGGAAGAGAAGGACATATGATAGTGATAAAGGATTAAGGAAATGATAG |
| | TATCATAGAAACATTGACTAGTACATGACCACTT |
| SEQ ID NO: 53 | TAGGGAAGAGAAGGACATATGATTAATGCACTTTGAACTTAAGCTATAAT |
| | AACTGATTAGTTGTTGACTAGTACATGACCACTT |
| SEQ ID NO: 54 | TAGGGAAGAGAAGGACATATGATCGTTTGGGCCTTATGTACGGCGTAAAT |
| | CTCTCCTGCAGAATTGACTAGTACATGACCACTT |
| SEQ ID NO: 55 | TAGGGAAGAGAAGGACATATGATAATGCGACATACCAATGTCGGACGACA |
| | ACAAGGCTAACATTTGACTAGTACATGACCACTT |
| SEQ ID NO: 56 | TAGGGAAGAGAAGGACATATGATAACGAACCGAATAGACCTGCGCGAAGA |
| | AAGGGTCTCAGAGTTGACTAGTACATGACCACTT |
| SEQ ID NO: 57 | TAGGGAAGAGAAGGACATATGATCGAGCGACACGAAAAGGGGCATGATCA |
| | TTGTCCATTGAAGTTGACTAGTACATGACCACTT |
| SEQ ID NO: 58 | TAGGGAAGAGAAGGACATATGATAATGACAAACAACCGCCTCACAGGTTT |
| | ACGGAACAAGACATTGACTAGTACATGACCACTT |
| SEQ ID NO: 59 | TAGGGAAGAGAAGGACATATGATCGTTTGGGCCTTATGTACGGCGTAAAT |
| | TTCTCCTGTAGAATTGACTAGTACATGACCACTT |
| SEQ ID NO: 60 | TAGGGAAGAGAAGGACATATGATTCTATCGGATCCAACGCGGATTTGAAT |
| | ATCAAGCGCAACGTTGACTAGTACATGACCACTT |
| SEQ ID NO: 61 | TAGGGAAGAGAAGGACATATGATCAGAAAGTCTGCGCAGCCAGACTGTGG |
| | GTAGACGACGCCATTGACTAGTACATGACCACTT |
| SEQ ID NO: 62 | TAGGGAAGAGAAGGACATATGATCGTTTGAGCCTTATGTACGGCGTAAAT |
| | TTCTCCTGCAGAATTGACTAGTACATGACCACTT |
| SEQ ID NO: 63 | TAGGGAAGAGAAGGACATATGATGAGGCTAGATGGACCAAGCCTCCTGAT |
| | CATAGTCCGAGAGTTGACTAGTACATGACCACTT |
| SEQ ID NO: 64 | TAGGGAAGAGAAGGACATATGATAGGCACAGCCTAGGAGATTCCTAGATT |
| | CCCGGAGGCATCTTTGACTAGTACATGACCACTT |
| SEQ ID NO: 65 | TAGGGAAGAGAAGGACATATGATCGTAGAGATGAGGCTTGCATTATTCGT |
| | TCCAAGCGATATGTTGACTAGTACATGACCACTT |
| SEQ ID NO: 66 | TAGGGAAGAGAAGGACATATGATCGTTTGGGCCTTATGTGCGGCGTAAAT |
| | TTCTCCTGCAGAATTGACTAGTACATGACCACTT |
| SEQ ID NO: 67 | TAGGGAAGAGAAGGACATATGATCGCTTGGGCCTTATGTACGGCGTAAAT |
| | TTCTCCTGCAGAATTGACTAGTACATGACCACTT |
| SEQ ID NO: 68 | TAGGGAAGAGAAGGACATATGATCGCGAAACGAAAAGGTTAGTCATCGCA |
| | TAGGAGACCGCCCTTGACTAGTACATGACCACTT |
| SEQ ID NO: 69 | TAGGGAAGAGAAGGACATATGATCGTCTGGGCCTTATGTACGGCGTAAAT |
| | TTCTCCTGCAGAATTGACTAGTACATGACCACTT |
| SEQ ID NO: 70 | TAGGGAAGAGAAGGACATATGATGTGTAAGAGAAGGAATAAAGTAGCGCT |
| | CAAGGTAAAGCAATTGACTAGTACATGACCACTT |
| SEQ ID NO: 71 | TAGGGAAGAGAAGGACATATGATCAGCTGGGCACGTTGATCATAGTACTT |
| | CGATGCACGGCGCTTGACTAGTACATGACCACTT |
| SEQ ID NO: 72 | TAGGGAAGAGAAGGACATATGATCGTTTGGGCCTTATGTATGGCGTAAAT |
| | TTCTCCTGCAGAATTGACTAGTACATGACCACTT |
| SEQ ID NO: 73 | TAGGGAAGAGAAGGACATATGATTGCATGGAGACAGACGCGGAGCGACCT |
| | CGGCACACATGAATTGACTAGTACATGACCACTT |
| SEQ ID NO: 74 | TAGGGAAGAGAAGGACATATGATGCTTCTATAAAAGAAGAACATAGAACG |
| | CATCAATTGGACCTTGACTAGTACATGACCACTT |
| SEQ ID NO: 75 | TAGGGAAGAGAAGGACATATGATGCGTGACAGCTAACACAGAATGAGAGA |
| | GGAAACGCACTAATTGACTAGTACATGACCACTT |
| SEQ ID NO: 76 | TAGGGAAGAGAAGGACATATGATTCACCTGAAATTTCCCAGGCTAAAATC |
| | ATATGGCCAACAGTTGACTAGTACATGACCACTT |
| SEQ ID NO: 77 | TAGGGAAGAGAAGGACATATGATCGCAACGTGGCAGTATACAGAAAACCG |
| | ATGCGAATGTGTCTTGACTAGTACATGACCACTT |
| SEQ ID NO: 78 | TAGGGAAGAGAAGGACATATGATCGTTTGGGCCTTATGTACGGCGTAAAT |
| | TTCTCCTGCGGAATTGACTAGTACATGACCACTT |
| SEQ ID NO: 79 | TAGGGAAGAGAAGGACATATGATAACGACAACCGAGAAGTAGCGAAAGAC |
| | AAACAACTTCTCATTGACTAGTACATGACCACTT |
| SEQ ID NO: 80 | TAGGGAAGAGAAGGACATATGATCGGGGCTGGCAAAAAACACAGGACCGA |
| | TCGTTGTCTCTGGTTGACTAGTACATGACCACTT |
| SEQ ID NO: 81 | TAGGGAAGAGAAGGACATATGATTCATAAAAGGTCAATTGCAGATCTAGT |
| | CTGCAGTGACTTATTGACTAGTACATGACCACTT |
| SEQ ID NO: 82 | TAGGGAAGAGAAGGACATATGATGGCATCATGTTCTTCGGCCAAGTTTCG |
| | CTTGCAAACCTTTTTGACTAGTACATGACCACTT |
| SEQ ID NO: 83 | TAGGGAAGAGAAGGACATATGATCGTTTGGGCCTTATGTACGGCGTGAAT |
| | TTCTCCTGCAGAATTGACTAGTACATGACCACTT |
| SEQ ID NO: 84 | TAGGGAAGAGAAGGACATATGATAGACGACAGAGGAGCCTATCAGCTGCC |
| | AATGACTAGTGACTTGACTAGTACATGACCACTT |
| SEQ ID NO: 85 | TAGGGAAGAGAAGGACATATGATTGCGTTTGTTGACACTCCTTTTCAAGG |
| | ATGCGTTGTCACCTTGACTAGTACATGACCACTT |
| SEQ ID NO: 86 | TAGGGAAGAGAAGGACATATGATGTATGGAGTCCGGGGAAACGGAGTCCA |
| | AAGCGAATCCCATTTGACTAGTACATGACCACTT |
| SEQ ID NO: 87 | TAGGGAAGAGAAGGACATATGATCGGTGACGCACGCAGGATTCCAAGGTC |
| | TCTGCCAAATCTATTGACTAGTACATGACCACTT |
| SEQ ID NO: 88 | TAGGGAAGAGAAGGACATATGATGCAGAAGGATGAAAGAGCACGAATCCA |
| | ACGATAATTGAAATTGACTAGTACATGACCACTT |
| SEQ ID NO: 89 | TAGGGAAGAGAAGGACATATGATACTCAACAAAAGAGGAAATCGATTAAG |
| | ACGCGACATACGTTTGACTAGTACATGACCACTT |
| SEQ ID NO: 90 | TAGGGAAGAGAAGGACATATGATCAAAAACGTAAGGATACAGTAACACAT |
| | ATGTAGAGGTTATTTGACTAGTACATGACCACTT |
| SEQ ID NO: 91 | TAGGGAAGAGAAGGACATATGATCGTGCTGAATTAGTGAGTGGTACACAC |
| | CGCCAGCATGATTTTGACTAGTACATGACCACTT |
| SEQ ID NO: 92 | TAGGGAAGAGAAGGACATATGATGTGTAAAGGAAATGTGGACCACACAAC |
| | CGCATTTCCGAAATTGACTAGTACATGACCACTT |
| SEQ ID NO: 93 | TAGGGAAGAGAAGGACATATGATGCCGCGAGAAGCCAACGACCACTCAGT |
| | CGATTGGTAGGGATTGACTAGTACATGACCACTT |
| SEQ ID NO: 94 | TAGGGAAGAGAAGGACATATGATGACGAAGTGTTGAAAGAGAAGGGCACC |
| | CAAACACTATCAATTGACTAGTACATGACCACTT |
| SEQ ID NO: 95 | TAGGGAAGAGAAGGACATATGATCCGGACAGGTCGAATCAACTGATCAAG |
| | GCCGGACTTACTGTTGACTAGTACATGACCACTT |
| SEQ ID NO: 96 | TAGGGAAGAGAAGGACATATGATGGACACAAAGGTAGAGACCTAGGATAT |
| | GGTCTCAAGCCAGTTGACTAGTACATGACCACTT |
| SEQ ID NO: 97 | TAGGGAAGAGAAGGACATATGATGCCGAACAACAAATTGGGCGGCAAATA |
| | AAAAGGATTTCCGTTGACTAGTACATGACCACTT |
| SEQ ID NO: 98 | TAGGGAAGAGAAGGACATATGATGAGATCGCAAAATGATGATAACGAACT |
| | TAGCAATCGCTAATTGACTAGTACATGACCACTT |
| SEQ ID NO: 99 | TAGGGAAGAGAAGGACATATGATACGCGCGGCCCTAGCACGCAAACAGTG |
| | AGACAAAGAATACTTGACTAGTACATGACCACTT |
| SEQ ID NO: 100 | TAGGGAAGAGAAGGACATATGATTTAACAGCCGCAATGAATATACAGGCG |
| | TATAAACATCTCATTGACTAGTACATGACCACTT |

TABLE 2
Sequences without Primer Recognition Sequences

| SEQ | |
|---|---|
| ID NO | Sequence |
| SEQ ID | CGTTTGGGCCTTATGTACGGCGTAAATTTCTCCTGCAGAA |
| NO: 101 | |
| SEQ ID | CCACTTATCTAGCGTAGATAAGGCGTTTAAAAGGTCTAAC |
| NO: 102 | |
| SEQ ID | TTCGGTCTAGCACACTCAACGAGATACTGGGGTTAAACGT |
| NO: 103 | |
| SEQ ID | CCCGATACTGAAAATGGAGGCCCGCAAGTATTATTTACAA |
| NO: 104 | |
| SEQ ID | CACTTCATGTAAGACTAAAAGATGGAGCGTGAAGGATGCA |
| NO: 105 | |
| SEQ ID | GTGGGTTAACTAATGAGGCTTAACGAGGCGTCAACGTTTT |
| NO: 106 | |
| SEQ ID | CGACACTGTTTGTAAGTCTTCCCTGATTACTTATTTCATC |
| NO: 107 | |
| SEQ ID | TTCCAGACGTTATTAGCCCGATCTCCTGTGTACGATCCAG |
| NO: 108 | |
| SEQ ID | GTATATGTACCAACCGAGTGATTCGGCCTATCAAAGCGTC |
| NO: 109 | |
| SEQ ID | CGTTTGGGCCCTATGTACGGCGTAAATTTCTCCTGCAGAA |
| NO: 110 | |
| SEQ ID | AAAAAACCGGGGTTCTTAATTTTCATTGTTCGTCGTACTT |
| NO: 111 | |
| SEQ ID | TACAAGTAAAACTGACCCCTCCATTTGTGTGTTTATTCGC |
| NO: 112 | |
| SEQ ID | ACCAATCATACTAAGCCTACCCGAACCAGCGGTGGAATGG |
| NO: 113 | |
| SEQ ID | CGTTTGGGCCTTACGTACGGCGTAAATTTCTCCTGCAGAA |
| NO: 114 | |
| SEQ ID | CGTTTGGGCCTTATGTACGGCGTAAATTTCTCCTGCAGAG |
| NO: 115 | |
| SEQ ID | TTAGTACGTATTGATATGATCTGAACTATGTGAGAATGAG |
| NO: 116 | |
| SEQ ID | CGTTTGGGCCTTATGTACGGCGTAAACTTCTCCTGCAGAA |
| NO: 117 | |
| SEQ ID | CCGTCTATTGCCGAGGATGGGTAATAGTACCGTGCGCACT |
| NO: 118 | |
| SEQ ID | CGTTTGGGCCTTATGTACAGCGTAAATTTCTCCTGCAGAA |
| NO: 119 | |
| SEQ ID | TGTTTGGGCCTTATGTACGGCGTAAATTTCTCCTGCAGAA |
| NO: 120 | |
| SEQ ID | CGTGAGGGAGACCCTACGAGAGAGAAAAGAAAAGGAAAAG |
| NO: 121 | |
| SEQ ID | CGTTTGGGCCTTATGTACGGCGTAAATTTCTCCTACAGAA |
| NO: 122 | |
| SEQ ID | ATAGAATGAGGAGGTCACCAATGGACACTAATCGACCGTA |
| NO: 123 | |
| SEQ ID | GGAGGAAAAGAGACAACAAAGAACGCCGCGCACAAGGCAC |
| NO: 124 | |
| SEQ ID | CGTTGATTTACGGTCGTAAGCGGTACGGTTTCATCGTCAG |
| NO: 125 | |
| SEQ ID | CGTTTGGGCCTTATGTACGGTGTAAATTTCTCCTGCAGAA |
| NO: 126 | |
| SEQ ID | CGTTTGGGCCTTATGTACGGCGTAAATTTCTCCCGCAGAA |
| NO: 127 | |
| SEQ ID | CGTTTGGGCCTTATGTACGGCGTAAATTTCCCCTGCAGAA |
| NO: 128 | |
| SEQ ID | CGTTTGGGCCTTATGTACGGCGTAAATTTCTCCTGCAGGA |
| NO: 129 | |
| SEQ ID | CGAGGGTCCTGTTGACTACGTCTTTGAACTCATTGGTCAC |
| NO: 130 | |
| SEQ ID | CGTTTAGGCCTTATGTACGGCGTAAATTTCTCCTGCAGAA |
| NO: 131 | |
| SEQ ID | CGTTTGGGCCTCATGTACGGCGTAAATTTCTCCTGCAGAA |
| NO: 132 | |
| SEQ ID | GGTCTAGAACAGAGATAACCAACATTGTCCCGAAAAGCCC |
| NO: 133 | |
| SEQ ID | TGCTGATCCCAGCAAACGGTATGACGCAACAGAGGTATCA |
| NO: 134 | |
| SEQ ID | GAGACAAATGATGTCCATGCATGCCGCCAAACAACCGAGA |
| NO: 135 | |
| SEQ ID | CCTTCAGAAAGGAACATATGCCGTTAAGACCAGAACTTCG |
| NO: 136 | |
| SEQ ID | CGTTTGGGCCTTATGCACGGCGTAAATTTCTCCTGCAGAA |
| NO: 137 | |
| SEQ ID | CACGGAAACGTGTAGAATTACACGTTAACGAAGTGAGGAG |
| NO: 138 | |
| SEQ ID | CGTTTGGGCCTTGTGTACGGCGTAAATTTCTCCTGCAGAA |
| NO: 139 | |
| SEQ ID | CAAACGTCGAATACGGATTGTCAAACGAACAACACCGTAT |
| NO: 140 | |
| SEQ ID | ATGGCCATCTGCCTAGTCATAGATTGGGAATCTGAACCGA |
| NO: 141 | |
| SEQ ID | GGCAGTCAGAAAGCTCTCGAGAATGTGGACCCGAGAGCAG |
| NO: 142 | |
| SEQ ID | GCCGTGTCGAGGAACATCCTAGACAAGGTGAAAAGTCCCA |
| NO: 143 | |
| SEQ ID | CAAGTAGCATAGTGGACGAACGAGCGGAACAAATGCGTAA |
| NO: 144 | |
| SEQ ID | TGCGATGCGAGTAGAAAAGCGTATGCTACAGGAACGTCCA |
| NO: 145 | |
| SEQ ID | TTTATCAGAGACGCGCCCTTAGCAAACGTGTTCTTCCGAA |
| NO: 146 | |
| SEQ ID | TATATGGAATCTCCAATGTGGCAACAGGACACATAAAGAA |
| NO: 147 | |
| SEQ ID | GCCGGATCAACCCAAGGAGTTGATTAGCATCATTTTACGA |
| NO: 148 | |
| SEQ ID | GTAGGGGTCCACGAAGTGCTAAGAAGGCACACATTTTGCG |
| NO: 149 | |
| SEQ ID | AACAGCGCGCGCCCTCAACGATAGACTATAAGTCCAAAGG |
| NO: 150 | |
| SEQ ID | GTACGCGTCAGAGTGTCGTGAACGACAAACGACTGATACC |
| NO: 151 | |
| SEQ ID | AGTGATAAAGGATTAAGGAAATGATAGTATCATAGAAACA |
| NO: 152 | |
| SEQ ID | TAATGCACTTTGAACTTAAGCTATAATAACTGATTAGTTG |
| NO: 153 | |
| SEQ ID | CGTTTGGGCCTTATGTACGGCGTAAATCTCTCCTGCAGAA |
| NO: 154 | |
| SEQ ID | AATGCGACATACCAATGTCGGACGACAACAAGGCTAACAT |
| NO: 155 | |
| SEQ ID | AACGAACCGAATAGACCTGCGCGAAGAAAGGGTCTCAGAG |
| NO: 156 | |
| SEQ ID | CGAGCGACACGAAAAGGGGCATGATCATTGTCCATTGAAG |
| NO: 157 | |
| SEQ ID | AATGACAAACAACCGCCTCACAGGTTTACGGAACAAGACA |
| NO: 158 | |
| SEQ ID | CGTTTGGGCCTTATGTACGGCGTAAATTTCTCCTGTAGAA |
| NO: 159 | |
| SEQ ID | TCTATCGGATCCAACGCGGATTTGAATATCAAGCGCAACG |
| NO: 160 | |
| SEQ ID | CAGAAAGTCTGCGCAGCCAGACTGTGGGTAGACGACGCCA |
| NO: 161 | |
| SEQ ID | CGTTTGAGCCTTATGTACGGCGTAAATTTCTCCTGCAGAA |
| NO: 162 | |
| SEQ ID | GAGGCTAGATGGACCAAGCCTCCTGATCATAGTCCGAGAG |
| NO: 163 | |
| SEQ ID | AGGCACAGCCTAGGAGATTCCTAGATTCCCGGAGGCATCT |
| NO: 164 | |
| SEQ ID | CGTAGAGATGAGGCTTGCATTATTCGTTCCAAGCGATATG |
| NO: 165 | |
| SEQ ID | CGTTTGGGCCTTATGTGCGGCGTAAATTTCTCCTGCAGAA |
| NO: 166 | |
| SEQ ID | CGCTTGGGCCTTATGTACGGCGTAAATTTCTCCTGCAGAA |
| NO: 167 | |
| SEQ ID | CGCGAAACGAAAAGGTTAGTCATCGCATAGGAGACCGCCC |
| NO: 168 | |
| SEQ ID | CGTCTGGGCCTTATGTACGGCGTAAATTTCTCCTGCAGAA |
| NO: 169 | |
| SEQ ID | GTGTAAGAGAAGGAATAAAGTAGCGCTCAAGGTAAAGCAA |
| NO: 170 | |
| SEQ ID | CAGCTGGGCACGTTGATCATAGTACTTCGATGCACGGCGC |
| NO: 171 | |
| SEQ ID | CGTTTGGGCCTTATGTATGGCGTAAATTTCTCCTGCAGAA |
| NO: 172 | |
| SEQ ID | TGCATGGAGACAGACGCGGAGCGACCTCGGCACACATGAA |
| NO: 173 | |
| SEQ ID | GCTTCTATAAAAGAAGAACATAGAACGCATCAATTGGACC |
| NO: 174 | |
| SEQ ID | GCGTGACAGCTAACACAGAATGAGAGAGGAAACGCACTAA |
| NO: 175 | |
| SEQ ID | TCACCTGAAATTTCCCAGGCTAAAATCATATGGCCAACAG |
| NO: 176 | |
| SEQ ID | CGCAACGTGGCAGTATACAGAAAACCGATGCGAATGTGTC |
| NO: 177 | |
| SEQ ID | CGTTTGGGCCTTATGTACGGCGTAAATTTCTCCTGCGGAA |
| NO: 178 | |
| SEQ ID | AACGACAACCGAGAAGTAGCGAAAGACAAACAACTTCTCA |
| NO: 179 | |
| SEQ ID | CGGGGCTGGCAAAAAACACAGGACCGATCGTTGTCTCTGG |
| NO: 180 | |
| SEQ ID | TCATAAAAGGTCAATTGCAGATCTAGTCTGCAGTGACTTA |
| NO: 181 | |
| SEQ ID | GGCATCATGTTCTTCGGCCAAGTTTCGCTTGCAAACCTTT |
| NO: 182 | |
| SEQ ID | CGTTTGGGCCTTATGTACGGCGTGAATTTCTCCTGCAGAA |
| NO: 183 | |
| SEQ ID | AGACGACAGAGGAGCCTATCAGCTGCCAATGACTAGTGAC |
| NO: 184 | |
| SEQ ID | TGCGTTTGTTGACACTCCTTTTCAAGGATGCGTTGTCACC |
| NO: 185 | |
| SEQ ID | GTATGGAGTCCGGGGAAACGGAGTCCAAAGCGAATCCCAT |
| NO: 186 | |
| SEQ ID | CGGTGACGCACGCAGGATTCCAAGGTCTCTGCCAAATCTA |
| NO: 187 | |
| SEQ ID | GCAGAAGGATGAAAGAGCACGAATCCAACGATAATTGAAA |
| NO: 188 | |
| SEQ ID | ACTCAACAAAAGAGGAAATCGATTAAGACGCGACATACGT |
| NO: 189 | |
| SEQ ID | CAAAAACGTAAGGATACAGTAACACATATGTAGAGGTTAT |
| NO: 190 | |
| SEQ ID | CGTGCTGAATTAGTGAGTGGTACACACCGCCAGCATGATT |
| NO: 191 | |
| SEQ ID | GTGTAAAGGAAATGTGGACCACACAACCGCATTTCCGAAA |
| NO: 192 | |
| SEQ ID | GCCGCGAGAAGCCAACGACCACTCAGTCGATTGGTAGGGA |
| NO: 193 | |
| SEQ ID | GACGAAGTGTTGAAAGAGAAGGGCACCCAAACACTATCAA |
| NO: 194 | |
| SEQ ID | CCGGACAGGTCGAATCAACTGATCAAGGCCGGACTTACTG |
| NO: 195 | |
| SEQ ID | GGACACAAAGGTAGAGACCTAGGATATGGTCTCAAGCCAG |
| NO: 196 | |
| SEQ ID | GCCGAACAACAAATTGGGCGGCAAATAAAAAGGATTTCCG |
| NO: 197 | |
| SEQ ID | GAGATCGCAAAATGATGATAACGAACTTAGCAATCGCTAA |
| NO: 198 | |
| SEQ ID | ACGCGCGGCCCTAGCACGCAAACAGTGAGACAAAGAATAC |
| NO: 199 | |
| SEQ ID | TTAACAGCCGCAATGAATATACAGGCGTATAAACATCTCA |
| NO: 200 | |

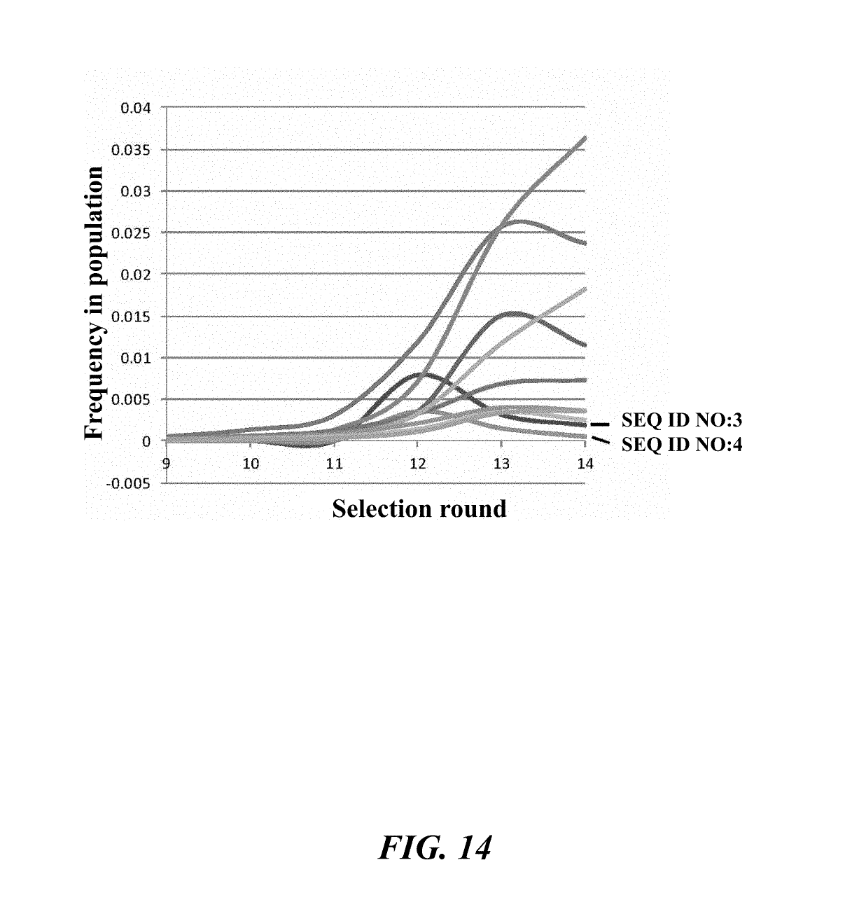

FIG. 14 illustrates a plot of the copy number of 10 different sequences in selection rounds 9-14 selected for twelve selection rounds for alternating parathyroid and thyroid tissues, and two subsequent selection negative selection rounds for thyroid tissue, plotted against the selection round. It can be seen in FIG. 14 that both SEQ ID NO:3 and SEQ ID NO:4 increase under positive selection conditions (selection round 12) and decrease in copy number (frequency) after two rounds of selection against thyroid tissue (selection rounds 13 and 14). Given that these sequences had the lowest copy number under negative selection conditions, they were further examined with the purpose of finding primary sequence or secondary/tertiary common elements.

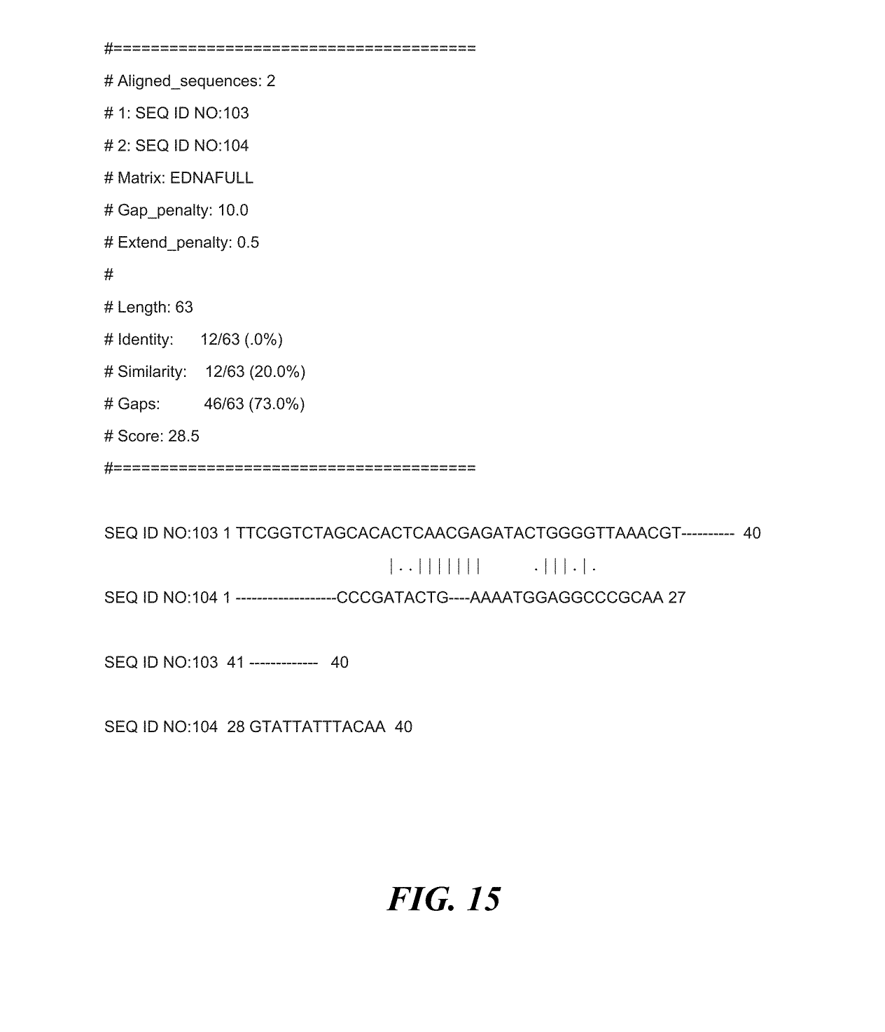

FIG. 15 illustrates a sequence alignment of SEQ ID NO:103 and SEQ ID NO:104. SEQ ID NO:103 and SEQ ID NO:104 are the variable regions of SEQ ID NO:3 and SEQ ID NO:4, respectively, without the common primer recognition sequences. The two sequences were aligned using the EMBOSS Needle pairwise nucleotide sequence alignment. The alignment, shown in FIG. 15, shows a common 5′-GATACTG-3′ motif in the two sequences which is underlined in the sequences for SEQ ID NO:3 and SEQ ID NO:4 in Table 1 and in the sequences for SEQ ID NO:103 and SEQ ID NO:104 in Table 2. SEQ ID NO:3 and SEQ ID NO:4 displayed high binding selectivity for human parathyroid tissue over human thyroid tissue. Based on analysis of the effect of positive selection for negative targets, it was determined that aptamer sequences SEQ ID NO:3 and SEQ ID NO:4 exhibited particularly promising specificity for binding to parathyroid tissue. SEQ ID NO:3 and SEQ ID NO:4 were tested on the same healthy human parathyroid slides to confirm that in fact they had high affinity for the parathyroid tissue that was used for positive selection. The results indicated that aptamers SEQ ID NO:3 and SEQ ID NO:4 both bind well to healthy human parathyroid.

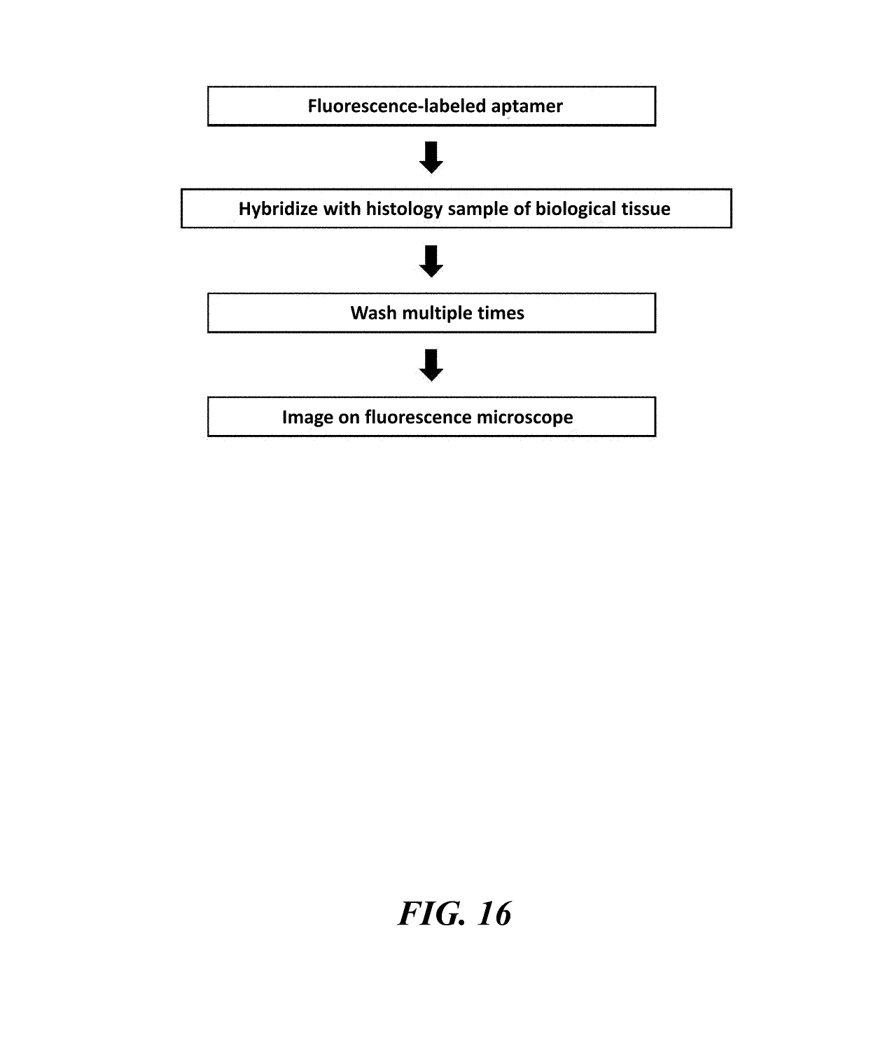

FIG. 16 shows the generic protocol for testing the specific binding of aptamers to tissue slides. 6-FAM Fluorescein-labeled aptamer at a concentration of 1 uM was allowed to bind to the tissue on the slide for half an hour and is then washed twice with PBS. This protocol can be tested by either using a single tissue slide as a target, or by using a tissue microarray, in which a number of diverse tissues are represented, for example in triplicate. This allows a competitive assay, were the labeled aptamer has the freedom to bind to any tissue. After stringent washing, only the tissues to which the aptamer is bound with high affinity will show a positive fluorescence signal.

### Example 2: Confirmation of Parathyroid Specificity

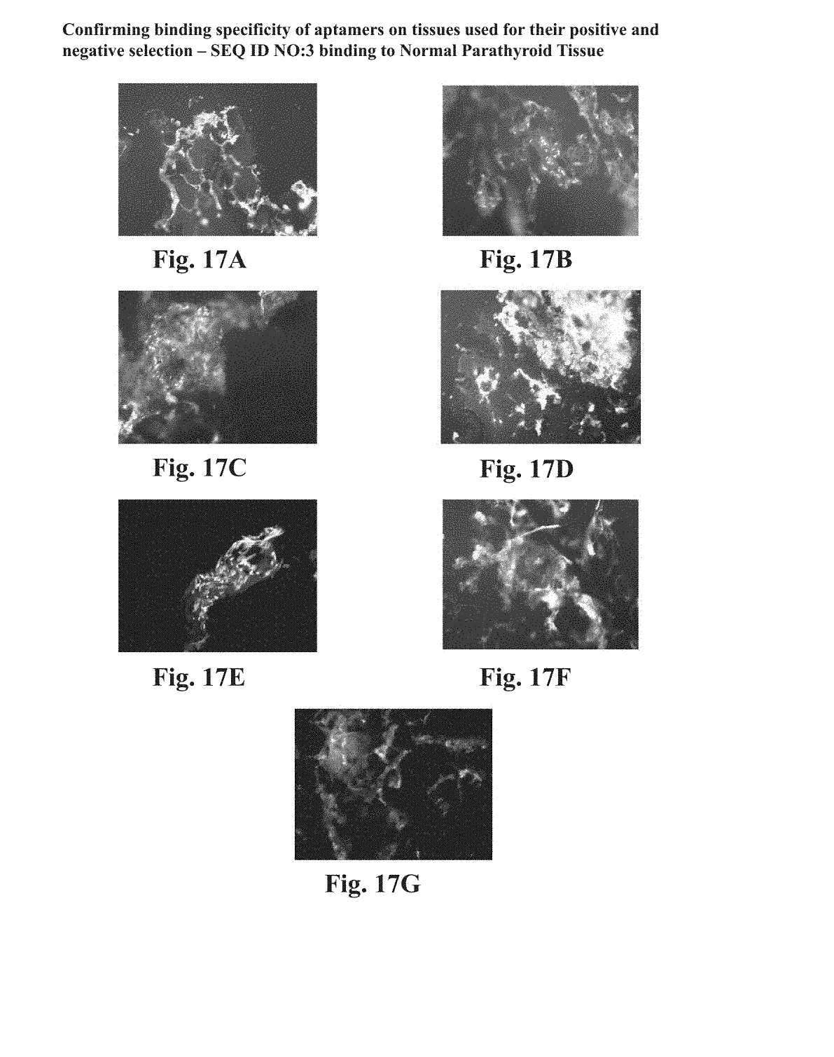

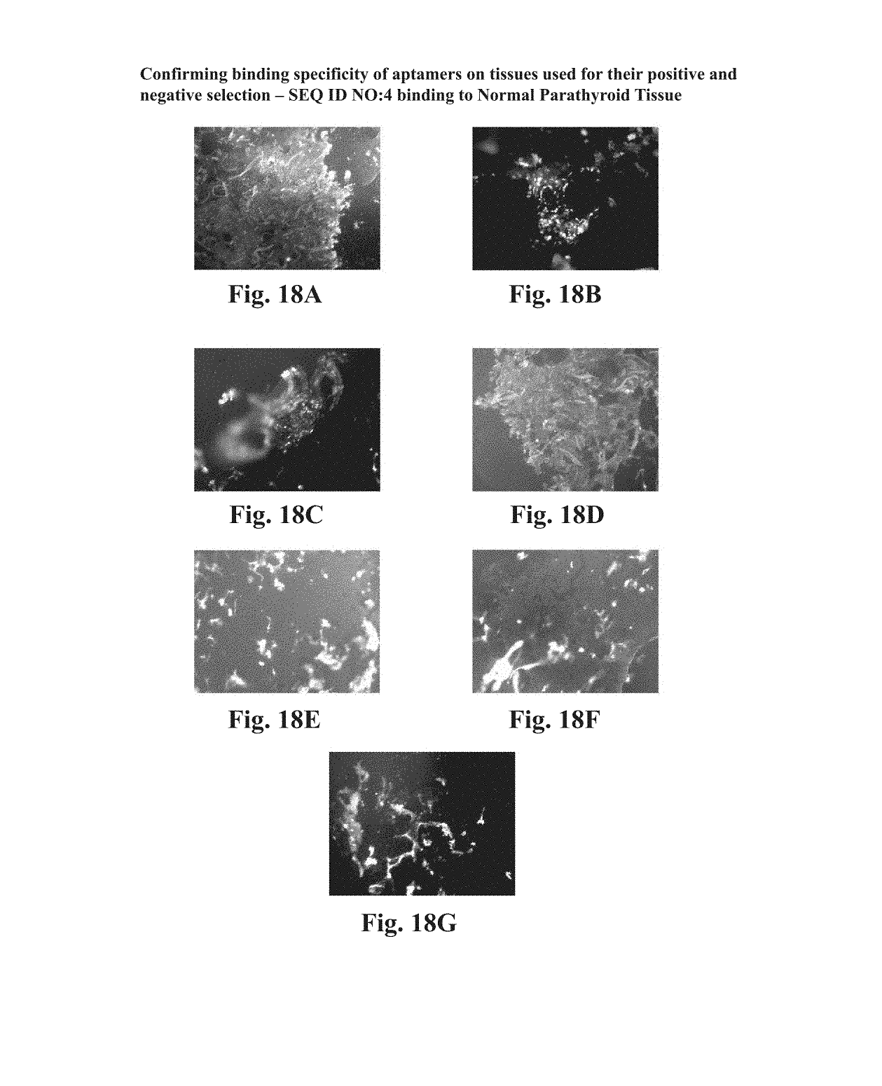

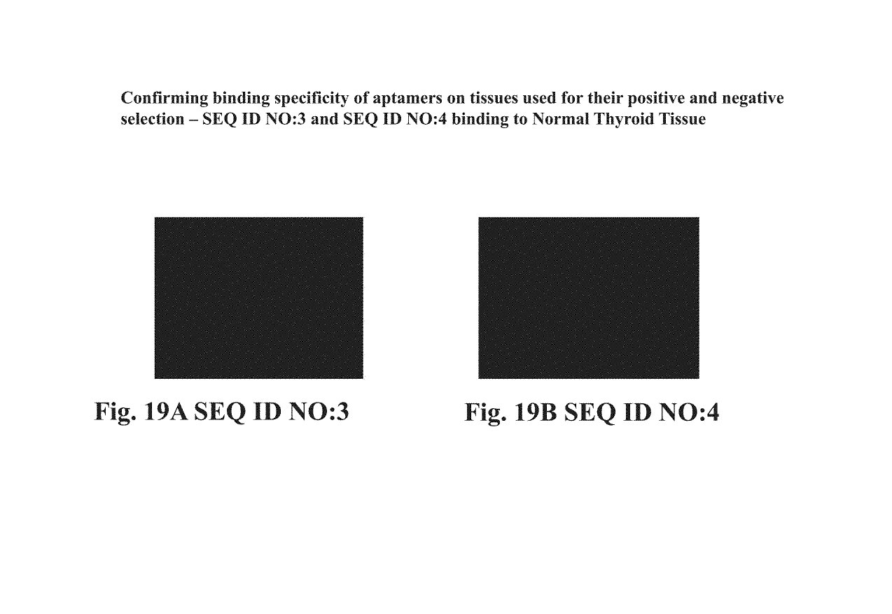

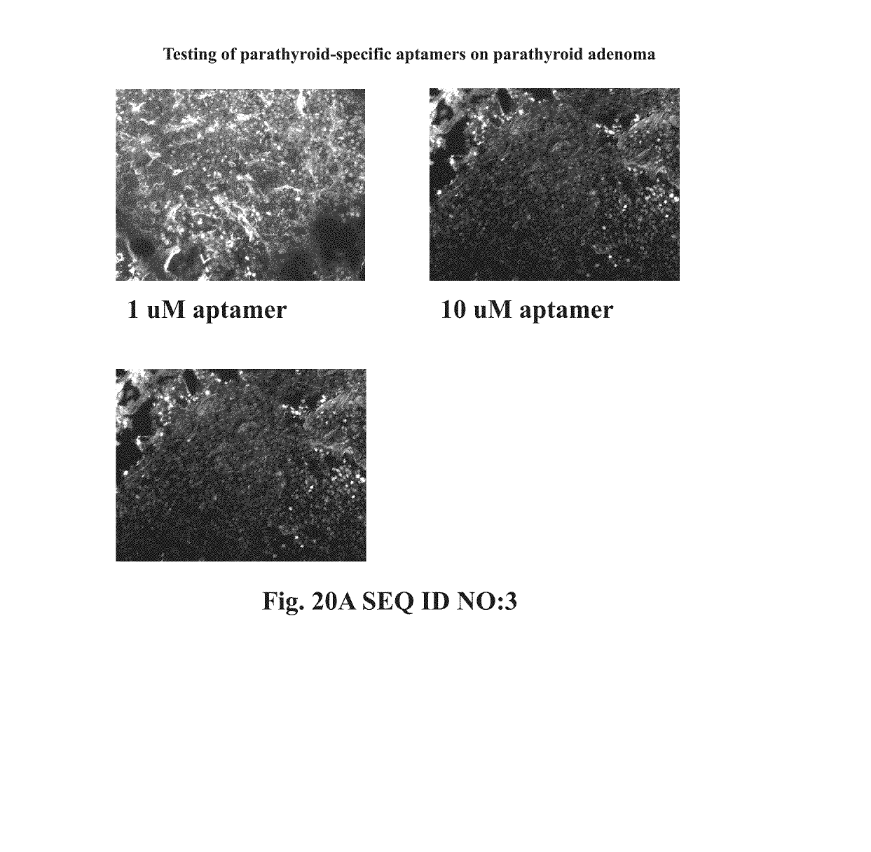

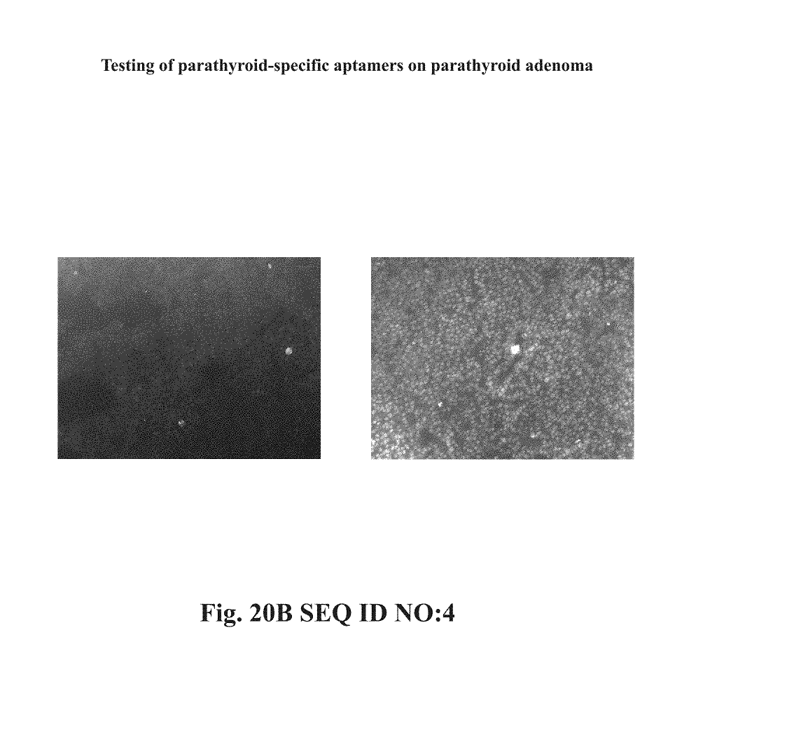

FIG. 17A-G confirms the binding of SEQ ID NO:3 to normal human parathyroid tissue slides, the same exact tissue that was used for its selection. FIG. 18A-G confirms the binding of SEQ ID NO:4 to normal human parathyroid tissue slides, identical to the slides used for its positive selection. FIG. 19 shows the lack of binding to normal thyroid tissue slides, similar slides to the ones used for the negative selection of the aptamers. FIG. 19A shows SEQ ID NO:3 binding to thyroid; FIG. 19B shows SED ID NO: 4 binding to normal thyroid. In order to test whether these aptamers bound not only normal parathyroid tissue but also affected parathyroid adenoma tissue, the binding of these two aptamers to parathyroid adenoma tissue slides was done. FIG. 20A and FIG. 20B show the results of the binding to parathyroid adenoma of SEQ ID NO:3 and SEQ ID NO:4, respectively.

### Example 3: Confirmation of Global Parathyroid Specificity

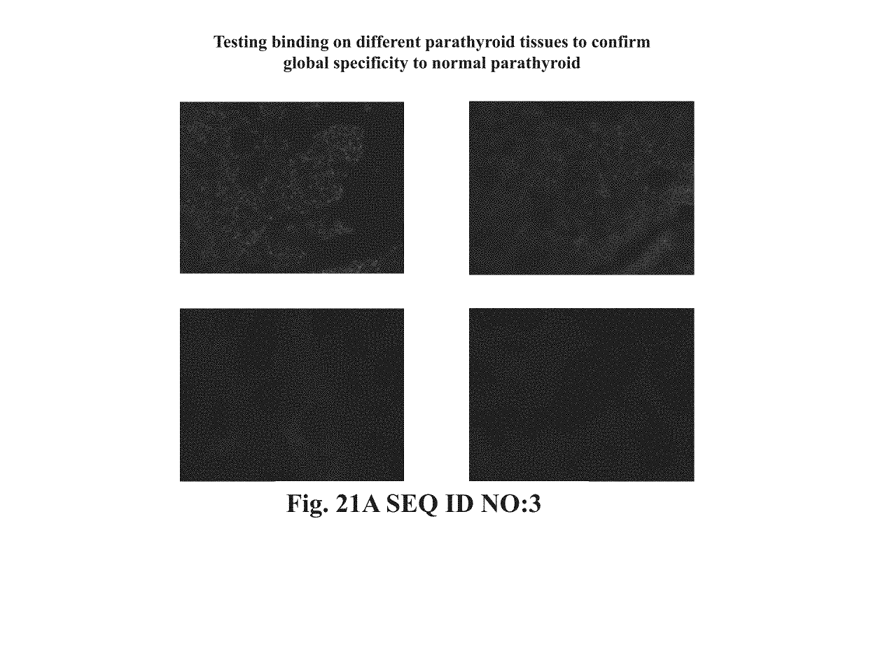

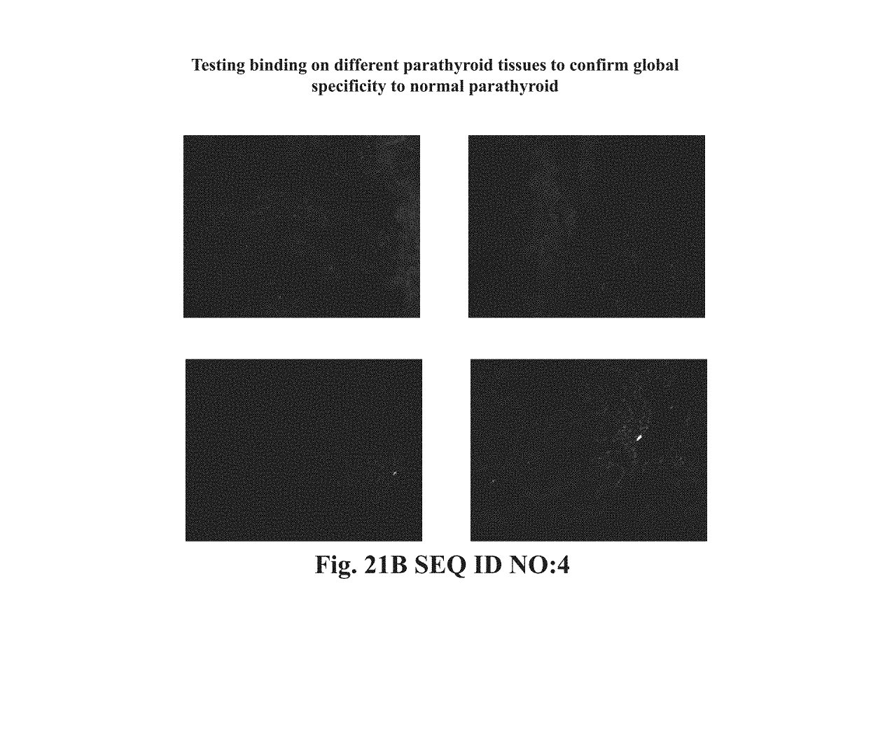

Additional testing was done to confirm that the parathyroid specificity that the aptamers were exhibiting was not the result of donor-specific determinants. Therefore, the aptamers were tested on additional parathyroid slides that originated from a completely different donor. FIGS. 21A and 21B show the results of the binding of SEQ ID NO:3 and SEQ ID NO:4 on two additional and unrelated parathyroid samples, respectively, proving that the aptamers were specifically targeting parathyroid and not donor-specific determinants.

### Example 4: Analysis of Aptamer Binding to Adipose, Lymph Node, Oropharynx, and Thymus Samples

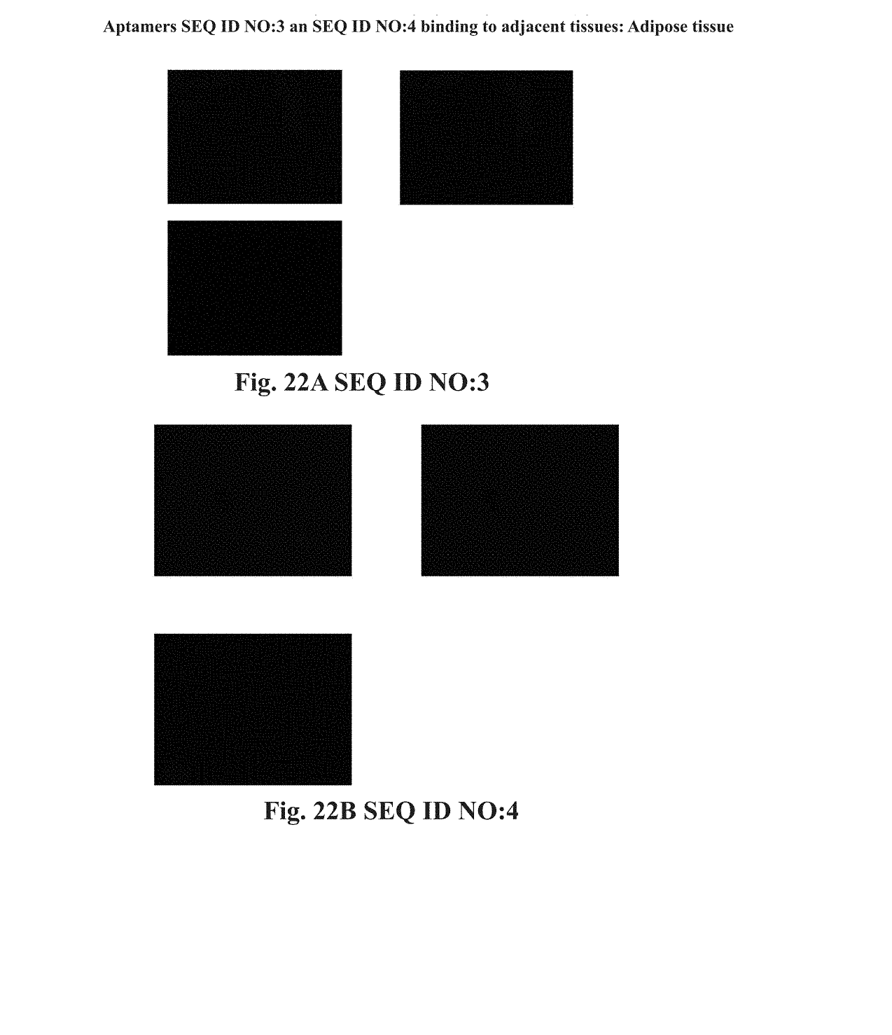

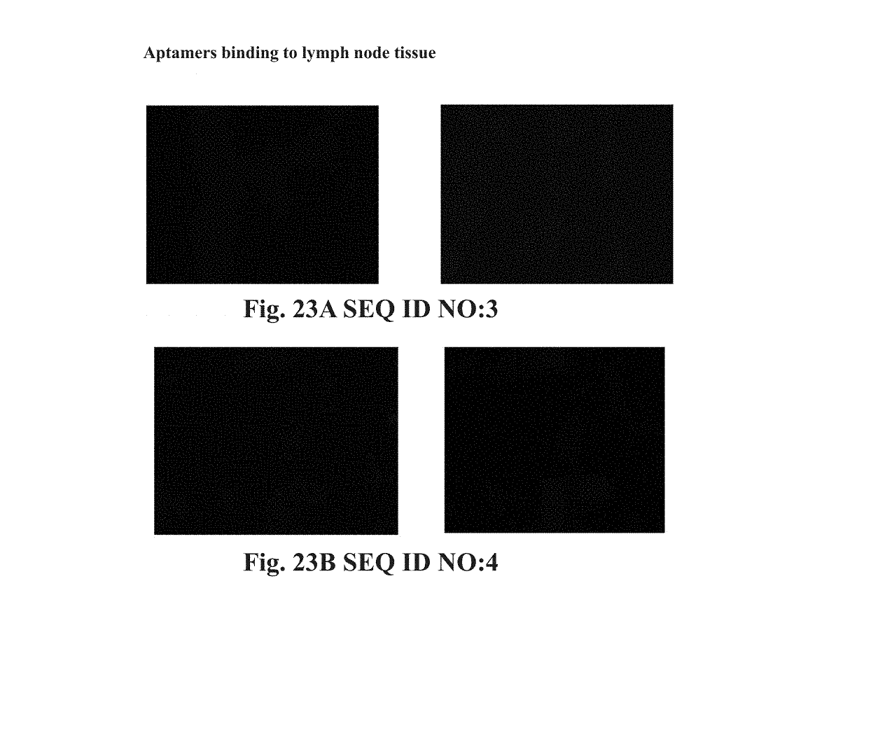

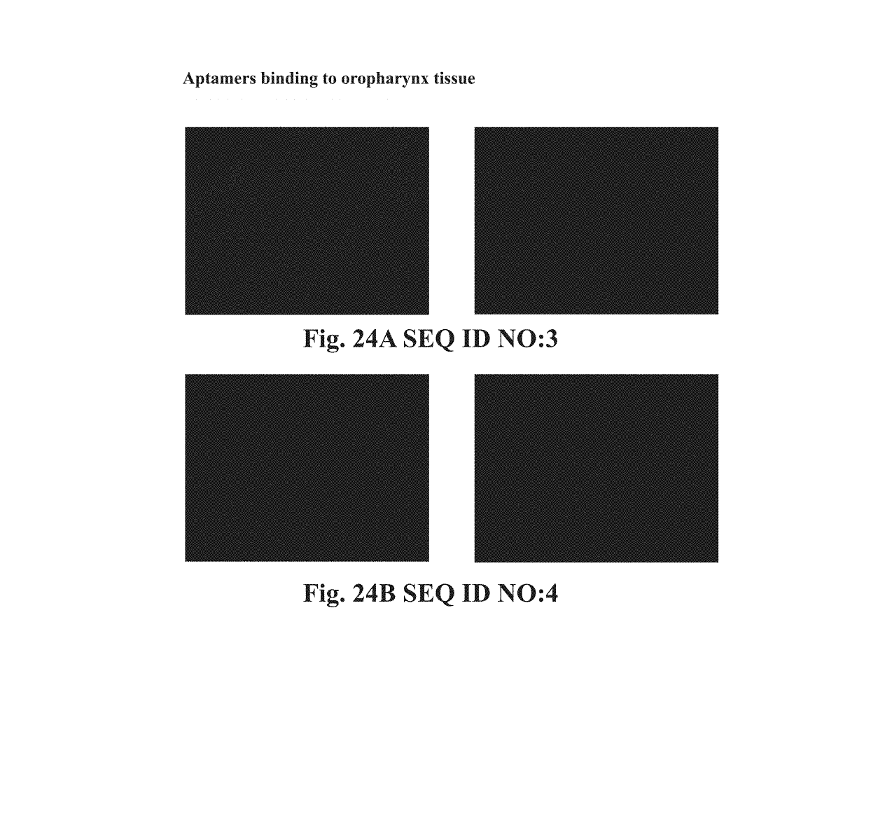

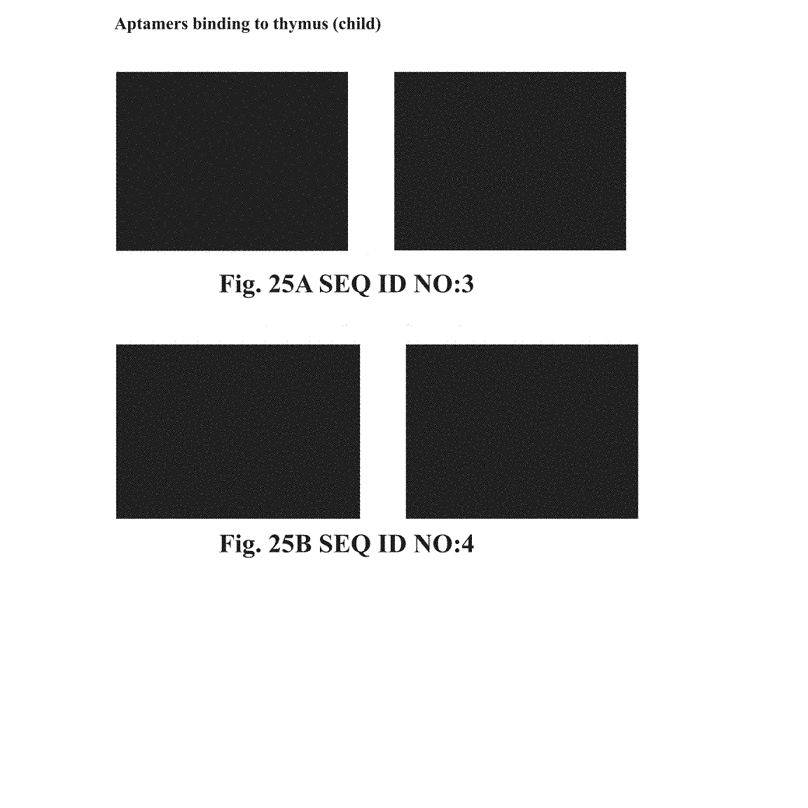

For the selected aptamers to work in a surgical thyroidectomy environment, for example, it is required that the aptamers not only bind specifically to their cognate target, but also exhibit very little, if any, binding to neighboring tissues. In this case, it is required that the aptamers exhibit minimal or no specific binding properties to fat, lymph nodes or thymus (in children). After binding and stringent washing, tissue samples were visualized on a microscope using FITC settings. Each tissue was controlled for background fluorescence to accurately determine specific binding versus no binding. FIGS. 22A and 22B display the binding of SEQ ID NO:3 and SEQ ID NO 4 to adipose tissue, respectively. Likewise, FIGS. 23A and 23B display the binding of SEQ ID NO:3 and SEQ ID NO 4 to lymph node tissue, respectively. FIGS. 24A and 24B illustrate the binding of SEQ ID NO:3 and SEQ ID NO 4 to oropharynx tissue, respectively. Lastly, FIGS. 25A and 25B illustrate the binding of SEQ ID NO:3 and SEQ ID NO 4 to child normal thymus tissue, respectively. The aptamers bound to all tested parathyroid samples, but consistently did not bind to thyroid, fat, thymus or oropharynx tissues.

### Example 5: Biodistribution: Analysis of Aptamers Binding to a Variety of Normal Human Tissue Samples

Fluorescent aptamer-based binding and differentiation of human tissues was tested using a normal tissue human specimen microarray. After aptamer binding and stringent washing, tissue samples were visualized on a microscope using FITC settings. Each tissue was controlled for background fluorescence to accurately determine specific binding versus no binding.

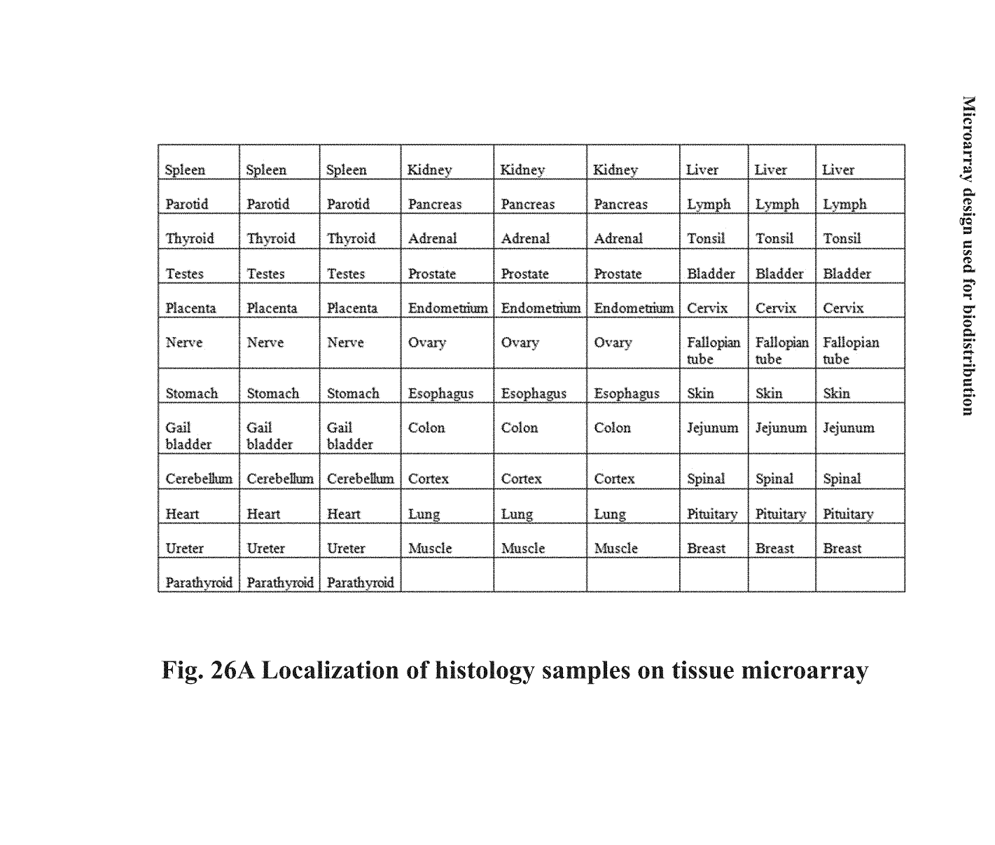

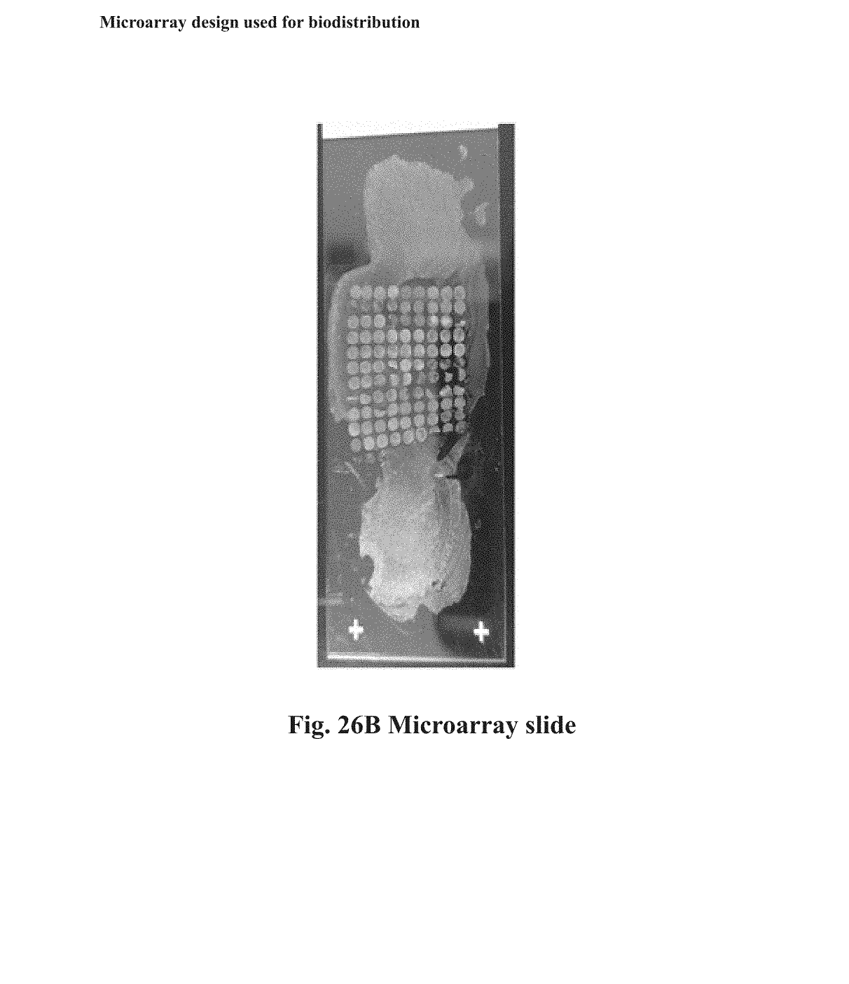

FIG. 26A maps the distribution and triplicate location of a large variety of normal human tissues on the tissue microarray slide. Data obtained from these binding assays can give an indication of the distribution of the aptamer in the patient's body once this marker is administered. In addition, tissue microarrays can be used to test in vitro bio-distribution of aptamers, indicator elements, and aptamers with bound indicator elements. The results from these studies can include measurements of the lack of autofluorescence. For example, human tissue microarrays as described previously can be used as a quick and efficient way to ensure that the tissue specific marker or aptamer binds selectively to the desired tissue of interest without substantial binding to any neighboring non-target tissues. It can additionally be used to assess the relative affinity of the labeled aptamer for each of the tissues represented.

The exemplary microarray described herein (FIGS. 26A-B) contained 34 different normal human tissues (each in triplicate) including parathyroid, thyroid, adrenal, bladder, brain cerebellum, brain cortex, breast, cervix, colon, endometrium, fallopian tube, gall bladder, heart, jejunum, kidney, liver, lung, lymph, muscle, nerve, esophagus, ovary, pancreas, parotid, pituitary, placenta, prostate, skin, spinal, spleen, stomach, testes, tonsil, and ureter tissues. Each of the three different fluorescently-labeled aptamers was tested on a microarray slide.

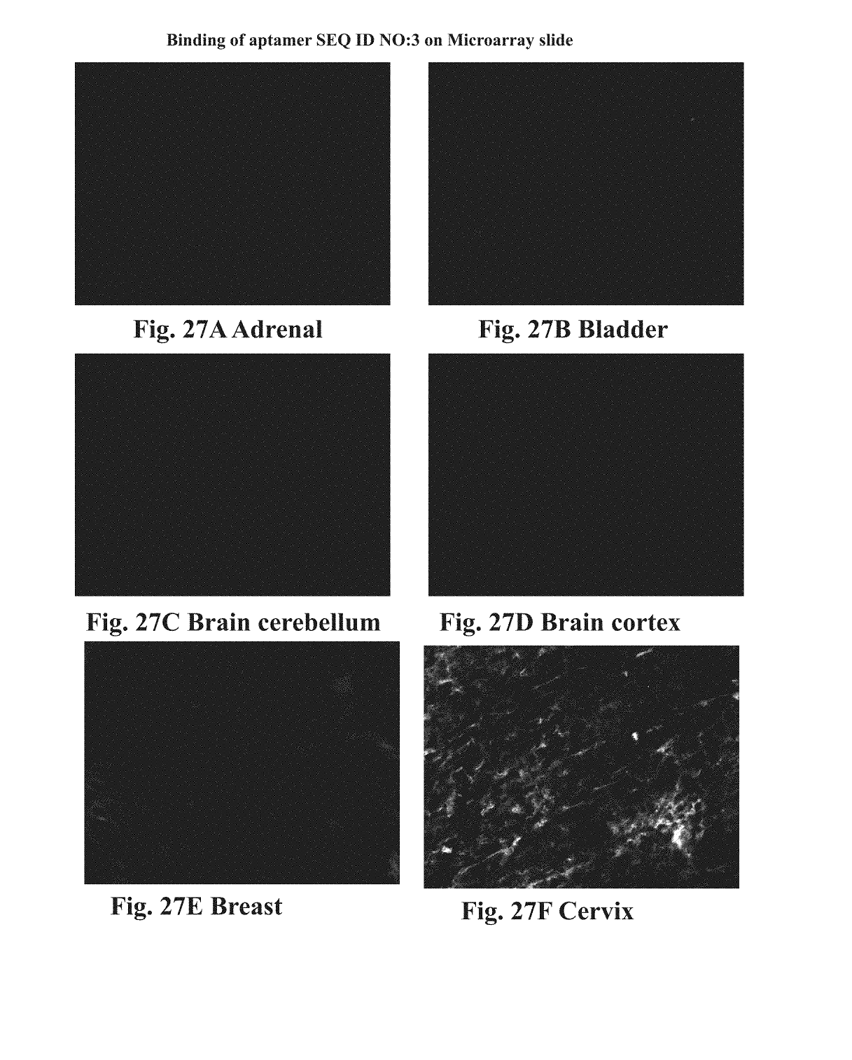

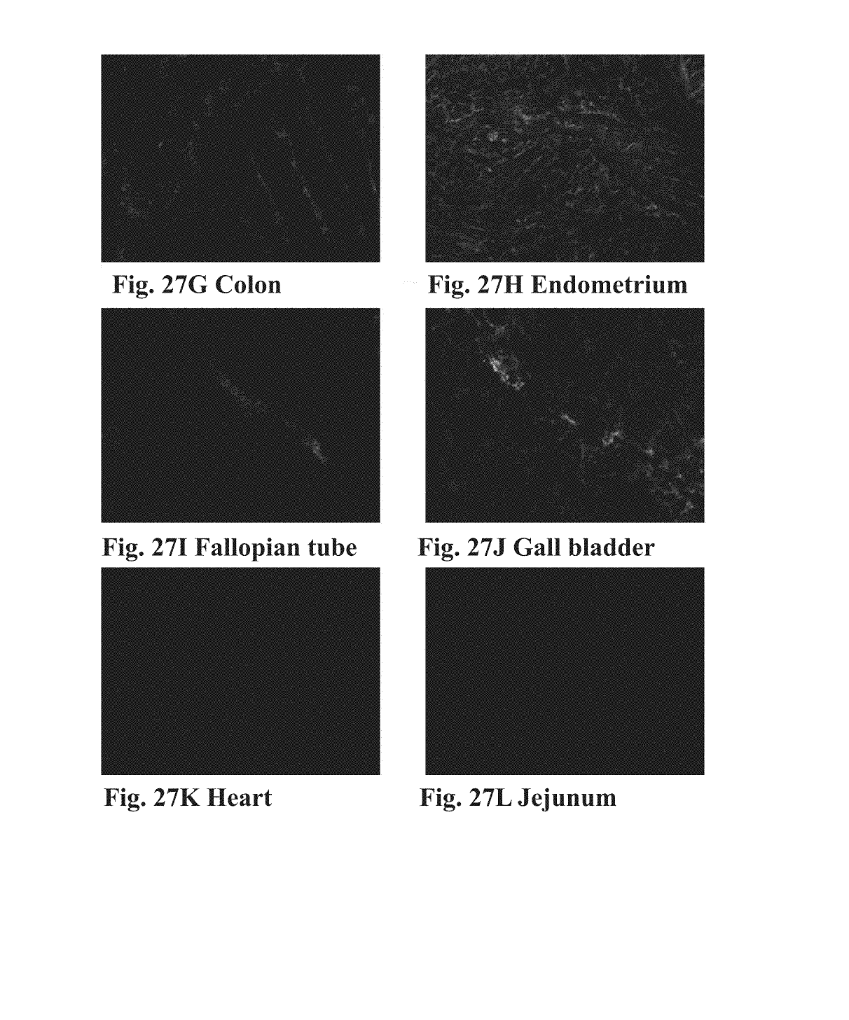

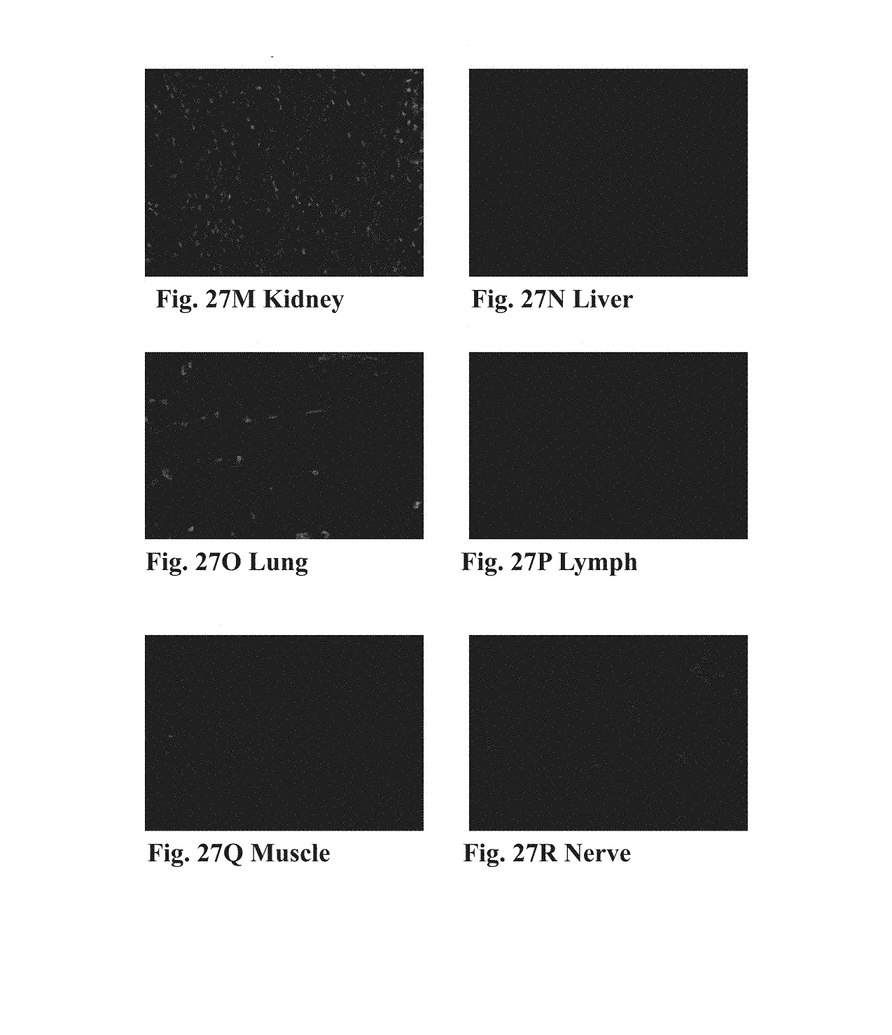

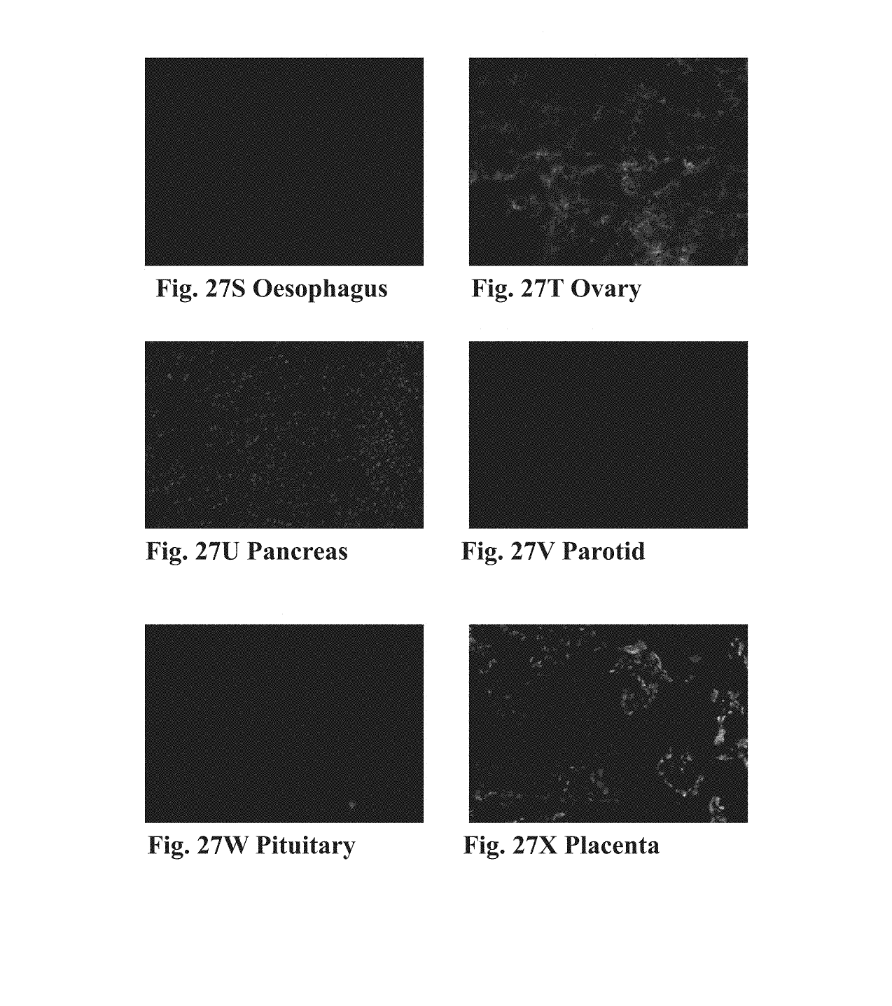

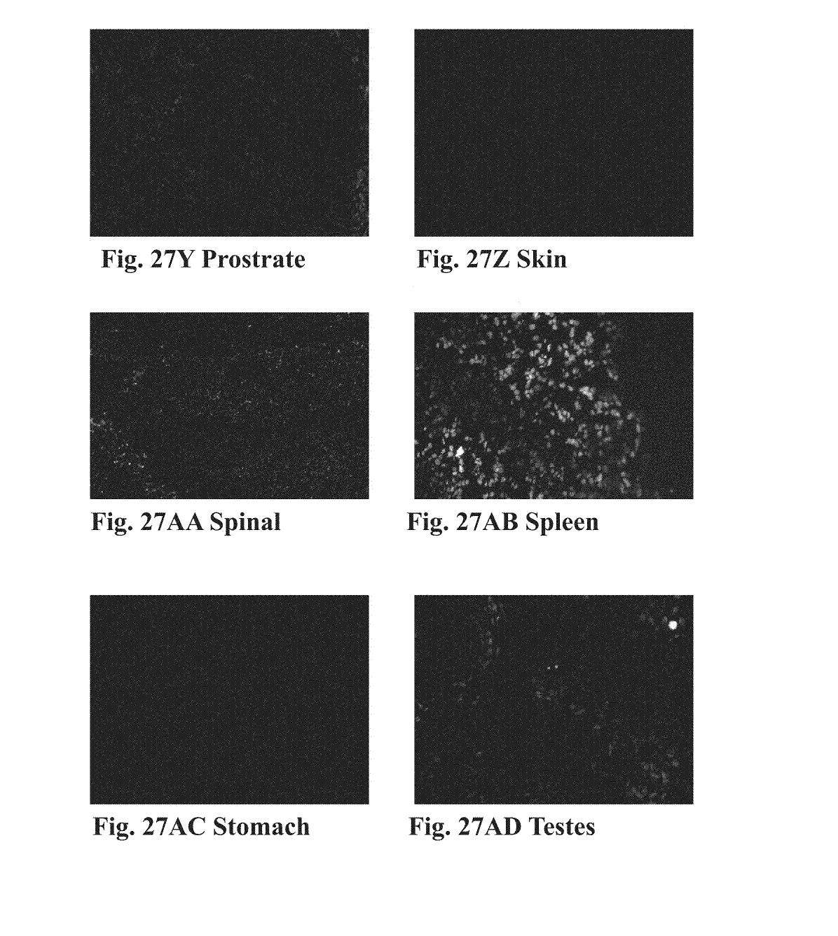

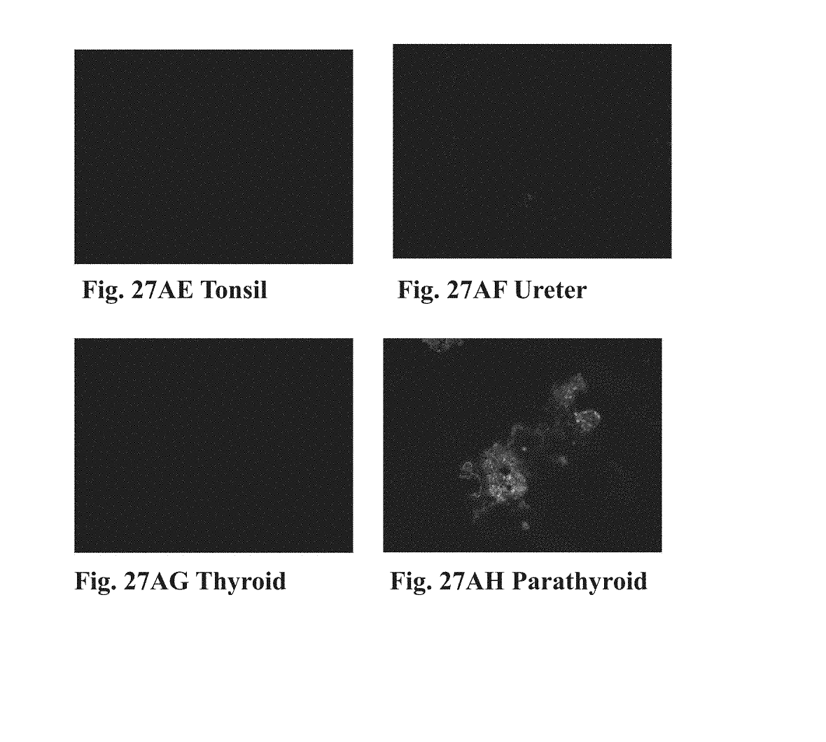

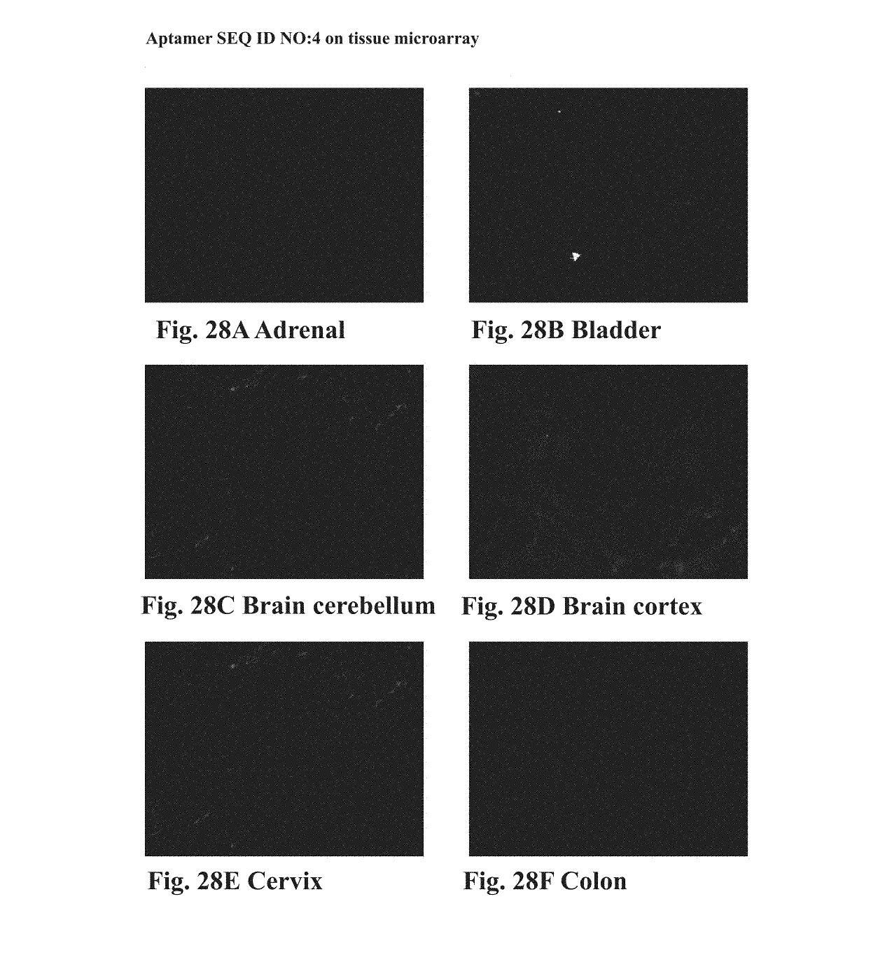

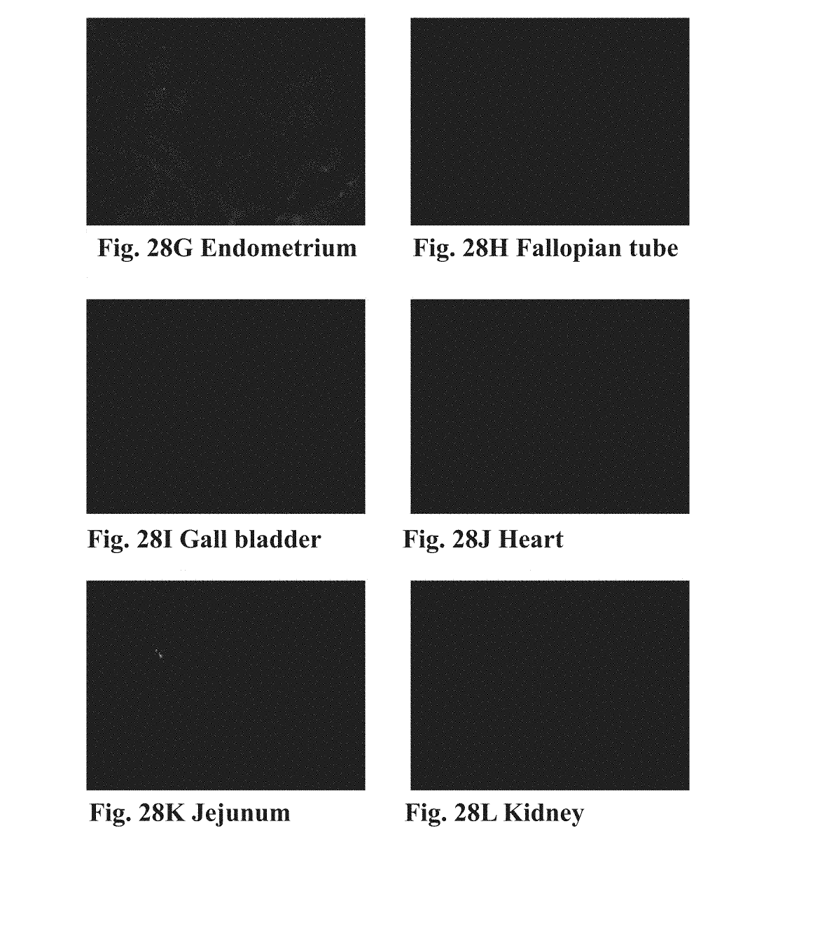

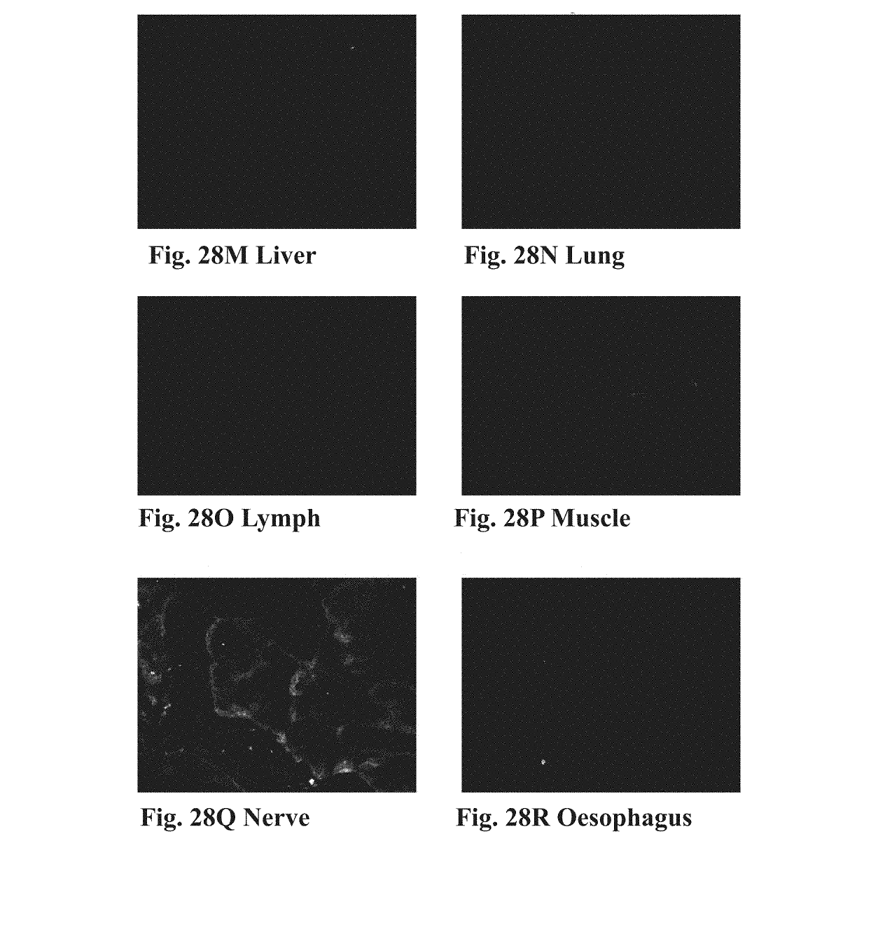

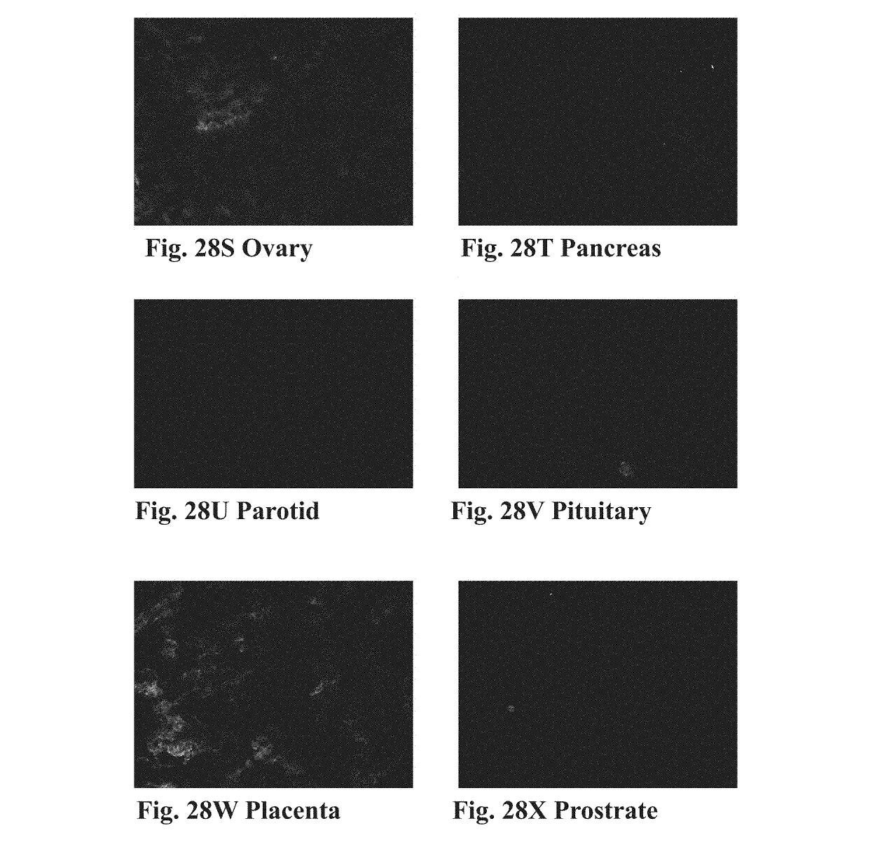

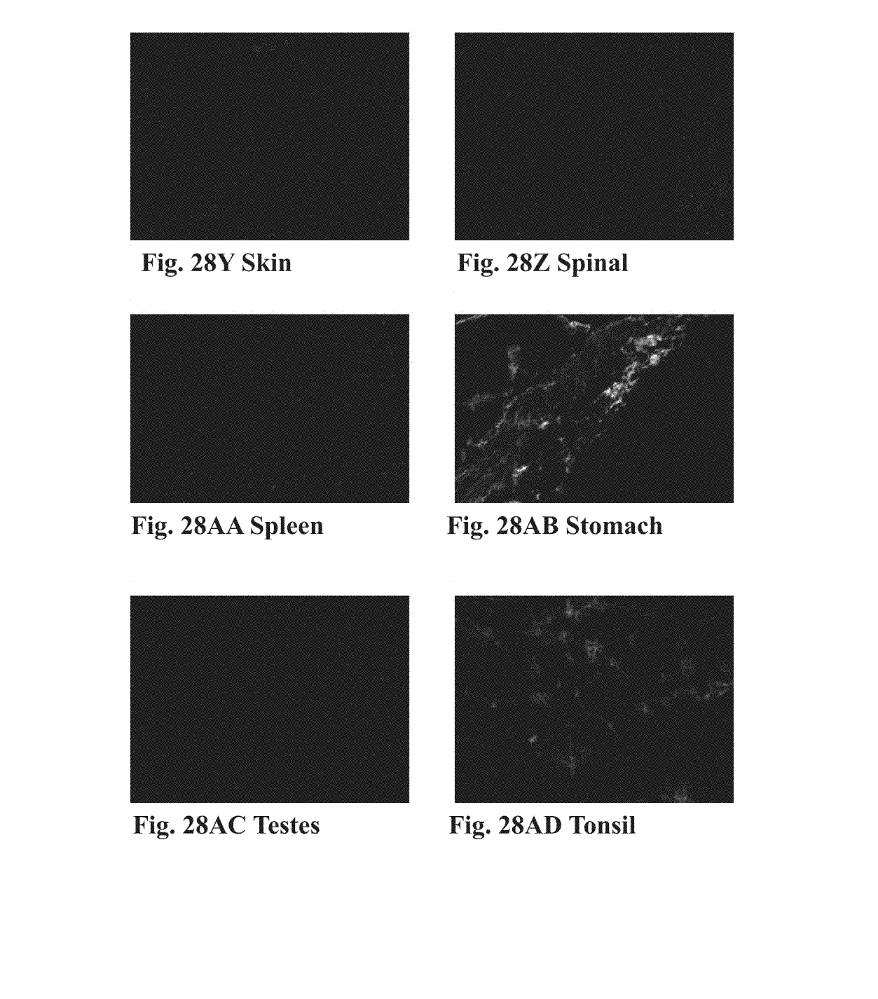

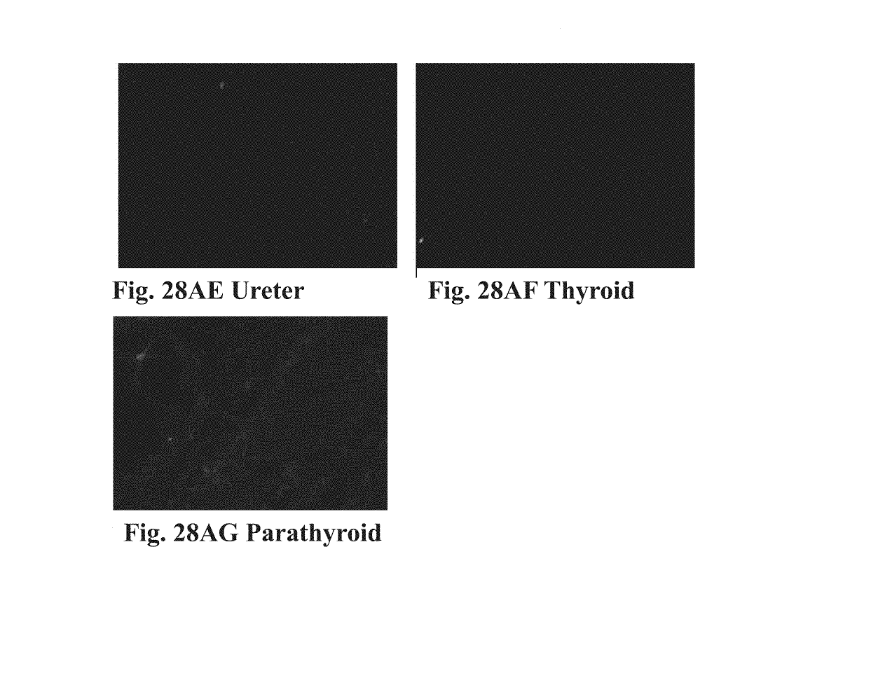

FIG. 27 and FIG. 28 show the binding of aptamers SEQ ID NO:3 SEQ ID NO:4 to the tissues represented on the microarray, respectively. The aptamers where labeled with 6-FAM Fluorescein at their 5′end and diluted at a concentration of 1 uM in PBS and allowed to bind to the microarray for 30 minutes, after which the slide was washed multiple times and images with a fluorescence-sensitive microscope. As can be seen in FIG. 27 for SEQ ID NO:3 and FIG. 28 for SEQ ID NO:4. The microarray studies confirmed that the aptamers selected bound to parathyroid tissue represented in the microarray with high affinity, but did not bind to the thyroid samples (see Table 3). Aptamers did not bind to the majority of tissues including: muscle, skin, brain, colon, liver, lymph node, etc. (see Table 3). Surprisingly, the tested aptamers bound to placenta and some to other female reproductive organs such as cervix, ovary, endometrium, etc. (see Table 3).

The selected aptamers behaved differently when binding to tissues such as nerve, where SEQ ID NO:4 appeared to bind better to the nerve tissue represented on the microarray than SEQ ID NO:3. It is possible that differences in background signal from tissue around the nerves may contribute to differences in binding detection. It is also likely that given the difference not only in primary sequence but also in tertiary structure, the aptamers have different binding specificities to other organs.

TABLE 3
MICROARRAY HISTOLOGY RESULTS

| | SEQ ID NO: 3 | | SEQ ID NO: 4 | |
|---|---|---|---|---|
| Tissue | Response | Notes | Response | Notes |
| Adrenal | − | | − | |
| Bladder | − | | + | minor binding, |
| | | | | bright |
| | | | | light is artifact |
| Brain | − | | + | |
| Cerebellum | | | | |
| Brain Cortex | − | | + | |
| Breast | + | | N/A | |
| Cervix | + | | + | |
| Colon | + | | − | |
| Endometrium | + | | + | |
| Fallopian Tube | + | | − | |
| Gall Bladder | + | | − | |
| Heart | − | | − | |
| Jejunum | − | | − | |
| Kidney | + | | − | |
| Liver | − | | − | |
| Lung | + | | − | |
| Lymph | − | | − | |
| Muscle | + | minor binding | − | |
| Nerve | + | minor binding | + | |
| Esophagus | − | | − | |
| Ovary | + | | + | |
| Pancreas | + | | + | |
| Parathyroid | + | | + | |
| Parotid | − | | − | |
| Pituitary | − | | + | minor binding, |
| | | | | bright |
| | | | | light is artifact |
| Placenta | + | | + | |
| Prostrate | + | | − | |
| Skin | − | | + | minor binding |
| Spinal | + | | + | |
| Spleen | + | | + | |
| Stomach | − | | + | |
| Testes | + | | − | |
| Thyroid | − | | − | |
| Tonsil | − | | + | |
| Ureter | + | minor binding | + | minor binding |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A tissue specific marker, the tissue specific marker comprising:
an aptamer or an affimer configured to selectively bind to a non-malignant target tissue; and
at least a first indicator element coupled to the aptamer or the affimer, wherein the at least a first indicator element produces a signal, thereby allowing identification of the non-malignant target tissue.

2. The tissue specific marker of claim 1, wherein the tissue specific marker comprises the aptamer configured to selectively bind to the non-malignant target tissue and wherein the aptamer configured to selectively bind to the non-malignant target tissue comprises DNA, RNA, a peptide, or any combination thereof.

3. The tissue specific marker of claim 1, wherein the tissue specific marker comprises the aptamer configured to selectively bind to the non-malignant target tissue and wherein the aptamer configured to selectively bind to the non-malignant target tissue comprises a nucleic acid.

4. The tissue specific marker of claim 3, wherein the aptamer configured to selectively bind to the non-malignant target tissue comprises modified nucleotides or nucleosides.

5. The tissue specific marker of claim 1, wherein the aptamer or the affimer configured to selectively bind to the non-malignant target tissue is PEGylated.

6. The tissue specific marker of claim 3, wherein the nucleic acid comprises an inverted T at a 3′ end of the nucleic acid.

7. The tissue specific marker of claim 3, wherein the aptamer has a length of up to 100 nucleotides.

8. The tissue specific marker of claim 2, wherein the aptamer comprises RNA or DNA, or both RNA and DNA.

9. The tissue specific marker of claim 1, wherein the aptamer binds to the non-malignant target tissue with an affinity within a range of 1 pM to 1 mM.

10. The tissue specific marker of claim 1, wherein the non-malignant tissue is a healthy tissue that is not an adipose tissue.

11. The tissue specific marker of claim 1, wherein the non-malignant tissue is not a healthy tissue.

12. The tissue specific marker of claim 2, wherein the aptamer selectively binds to the non-malignant target tissue with an affinity at least 2-fold higher than an affinity of the aptamer binding to a non-target tissue.

13. The tissue specific marker of claim 1, wherein the non-malignant target tissue comprises a parathyroid gland or a parathyroid adenoma.

14. The tissue specific marker of claim 1, wherein the non-malignant target tissue comprises parathyroid tissue.

15. The tissue specific marker of claim 1, wherein the non-malignant target tissue is a human tissue.

16. The tissue specific marker of claim 1, wherein the non-malignant target tissue is a gland tissue.

17. The tissue specific marker of claim 16, wherein the gland tissue is an exocrine gland tissue or an endocrine gland tissue.

18. The tissue specific marker of claim 1, wherein the aptamer or the affimer is configured to selectively bind to a first gland over a second gland.

19. The tissue specific marker of claim 1, wherein the aptamer or the affimer is configured to preferentially bind the non-malignant target tissue over a tissue situated adjacent to the non-malignant target tissue.

20. The tissue specific marker of claim 16, wherein the aptamer or the affimer is configured to preferentially bind the gland tissue over at least one tissue selected from the group consisting of adipose, thymus, lymph node and pharynx tissue.

21. The tissue specific marker of claim 16, wherein the aptamer or the affimer is configured to selectively bind to a healthy parathyroid tissue or a parathyroid adenoma tissue, or both.

22. The tissue specific marker of claim 21, wherein the aptamer or the affimer preferentially binds to the healthy parathyroid tissue or the parathyroid adenoma tissue over a thyroid tissue.

23. The tissue specific marker of claim 21, wherein the aptamer or the affimer preferentially binds to the healthy parathyroid tissue or the parathyroid adenoma tissue over at least one tissue selected from the group consisting of adipose, thymus, lymph node and pharynx tissue.

24. The tissue specific marker of claim 1, wherein the non-malignant target tissue comprises a nerve, a blood vessel, a ureter, a bile duct, endometrial tissue, hepatic duct, lymph nodes, bacteria or fungus.

25. The tissue specific marker of claim 1, wherein the non-malignant target tissue comprises female reproductive tissues such as endometrium and uterus.

26. The tissue specific marker of claim 21, wherein the aptamer comprises a sequence with at least 70% sequence identity to any one of SEQ ID NOs: 1-200.

27. The tissue specific marker of claim 21, wherein the aptamer comprises a sequence with at least 70% sequence identity to any one of SEQ ID NOs: 1-10 and SEQ ID NOs: 100-110.

28. The tissue specific marker of claim 21, wherein the aptamer comprises a sequence with at least 70% sequence identity to SEQ ID NO:1, SEQ ID NO:2, SEQ ID NO:3, SEQ ID NO:4, SEQ ID NO:5, SEQ ID NO:101, SEQ ID NO:102, SEQ ID NO:103, SEQ ID NO:104, or SEQ ID NO:105.

29. The tissue specific marker of claim 21, wherein the aptamer comprises a sequence of SEQ ID NO:3, SEQ ID NO:4, SEQ ID NO:103, or SEQ ID NO:104.

30. The tissue specific marker of claim 21, wherein the aptamer comprises a sequence that includes a motif GATACTG.

31. The tissue specific marker of claim 21 wherein the at least a first indicator element produces the signal when exposed to or excited by energy.

32. The tissue specific marker of claim 21, wherein the signal produced is within a magnetic, acoustic, visible, near-infrared, or infrared spectrum.

33. The tissue specific marker of claim 1, wherein the at least a first indicator element comprises a fluorophore.

34. The tissue specific marker of claim 33, wherein the fluorophore is a near-infrared dye.

35. The tissue specific marker of claim 33, wherein the fluorophore is a cyanine dye or indocyanine green.

36. The tissue specific marker of claim 1, wherein the at least a first indicator element comprises an element selected from the group consisting of: quantum dot, nanodiamond, enzyme, protein, nanorod, nanoparticle, an optoacoustic converter element, bead, and nanocrystal.

37. The tissue specific marker of claim 1, wherein the at least a first indicator element is covalently coupled to the aptamer, the affimer, or a second indicator element.

38. The tissue specific marker of claim 1, wherein the at least a first indicator element comprises a first indicator element that is a fluorophore and a second indicator element that is a nanodiamond.

39. A system for differentiating a target tissue from an adjacent tissue, said system comprising:
the tissue specific marker of claim 1; and
a device for exciting the tissue specific marker with energy or a detector for detecting the signal from the one or more indicator elements.

40. A system for differentiating a target tissue from an adjacent tissue, said system comprising:
the tissue specific marker of claim 1;
a second tissue specific marker, comprising a second aptamer or a second affimer configured to bind to a second target tissue; and a second indicator element coupled with the second aptamer or the second affimer, wherein the second indicator element produces a signal thereby allowing identification of the second target tissue; and
a first device for exciting the tissue specific marker with energy or a detector for detecting the signal from any of the first or second indicator elements, or from both the first and second indicator elements.

41. A method for differentiating tissue, said method comprising:
delivering an aptamer or an affimer coupled to one or more indicator elements into a subject's body;
allowing the aptamer or the affimer to bind with a normal or non-malignant target tissue in the subject's body;
detecting a signal produced by the one or more indicator elements or detecting the one or more indicator elements; and
distinguishing the normal or non-malignant target tissue from an adjacent tissue based on the signal detected from the one or more indicator elements.

42. The method of claim 41, wherein delivering the aptamer or the affimer comprises transdermally delivering, spraying, flooding, orally delivering, or intravenously delivering the aptamer or the affimer to the non-malignant target tissue.

43. The method of claim 41, further comprising exposing or exciting the one or more indicator elements that are coupled to the aptamer or the affimer with energy.

44. The method of claim 41, wherein detecting the signal from the one or more indicator elements comprises detecting a spectral signal with a detector.

45. The method of claim 41, wherein detecting the signal from the one or more indicator elements comprises detecting a spectral signal with a camera.

46. The method of claim 41, further comprising performing a medical procedure on the non-malignant target tissue without damaging the adjacent tissue.

47. The method of claim 41, wherein the non-malignant target tissue is a parathyroid adenoma and the method further comprises removing at least a portion of the parathyroid adenoma without damaging an adjacent thyroid tissue.

48. The method of claim 41, wherein the non-malignant target tissue is a parathyroid tissue, and the method further comprises removing at least a portion of the adjacent thyroid tissue without damaging or removing the parathyroid tissue.

49. The method of claim 41, wherein the aptamer or the affimer selectively binds to both the healthy parathyroid tissue and the parathyroid adenoma tissue over the adjacent thyroid tissue.

50. The method of claim 41, further comprising detecting a first set of the one or more indicator elements and a second set of the one or more indicator elements, wherein the first set is detected at a greater distance from the tissue specific marker than the second set.

51. The method of claim 50, wherein the first set is detected outside the subject's body and the second set is detected after a surgical incision is made.

52. The method of claim 41, further comprising forming a diagnosis based on the detected spectral signal.

53. The method of claim 41, wherein detecting the signal from the one or more indicator elements comprises detecting the signal preoperatively.

54. The method of claim 41, wherein detecting the signal from the one or more indicator elements comprises detecting the signal intraoperatively.

55. The method of claim 41, further comprising:
delivering a second aptamer or a second affimer coupled to the one or more indicator elements into the subject's body;
allowing the second aptamer or the second affimer to bind with a second target tissue, wherein the second target tissue is different than the normal or non-malignant target tissue;
exposing or exciting the one or more indicator elements that are coupled to the second aptamer or the second affimer with energy;
detecting a spectral signal produced by the one or more indicator elements that are coupled to the second aptamer or the second affimer or detecting the one or more indicator elements that are coupled to the second aptamer or the second affimer; and
identifying the second target tissue from the normal or non-malignant target tissue based on the spectral signal detected from the one or more indicator elements that are coupled to the second aptamer or the second affimer.

56. An aptamer that selectively binds to a parathyroid gland or a parathyroid adenoma.

57. The aptamer of claim 56, wherein the aptamer selectively binds to the parathyroid gland or the parathyroid adenoma with an affinity at least 10-fold higher than an affinity of the aptamer for binding to a thyroid gland.

58. A polynucleotide comprising a sequence with at least 70% sequence identity to any one of SEQ ID NOs: 1-200, wherein the sequence comprises a non-natural sequence of at least ten contiguous nucleotides or the sequence comprises at least one modified nucleotide.


SEQUENCE LISTING (202 sequences)

SEQ ID NO: 1 - DNA, 84 bp, Artificial Sequence
  Description of Artificial Sequence Synthetic oligonucleotide
tagggaagagaaggacatatgatcgtttgggccttatgtacggcgtaaatttctcctgcagaattgactagtacatgaccactt

SEQ ID NO: 2 - DNA, 84 bp, Artificial Sequence
  Description of Artificial Sequence Synthetic oligonucleotide
tagggaagagaaggacatatgatccacttatctagcgtagataaggcgtttaaaaggtctaacttgactagtacatgaccactt

SEQ ID NO: 3 - DNA, 84 bp, Artificial Sequence
  Description of Artificial Sequence Synthetic oligonucleotide
tagggaagagaaggacatatgatttcggtctagcacactcaacgagatactggggttaaacgtttgactagtacatgaccactt

SEQ ID NO: 4 - DNA, 84 bp, Artificial Sequence
  Description of Artificial Sequence Synthetic oligonucleotide
tagggaagagaaggacatatgatcccgatactgaaaatggaggcccgcaagtattatttacaattgactagtacatgaccactt

SEQ ID NO: 5 - DNA, 84 bp, Artificial Sequence
  Description of Artificial Sequence Synthetic oligonucleotide
tagggaagagaaggacatatgatcacttcatgtaagactaaaagatggagcgtgaaggatgcattgactagtacatgaccactt

SEQ ID NO: 6 - DNA, 84 bp, Artificial Sequence
  Description of Artificial Sequence Synthetic oligonucleotide
tagggaagagaaggacatatgatgtgggttaactaatgaggcttaacgaggcgtcaacgttttttgactagtacatgaccactt

SEQ ID NO: 7 - DNA, 84 bp, Artificial Sequence
  Description of Artificial Sequence Synthetic oligonucleotide
tagggaagagaaggacatatgatcgacactgtttgtaagtcttccctgattacttatttcatcttgactagtacatgaccactt

SEQ ID NO: 8 - DNA, 84 bp, Artificial Sequence
  Description of Artificial Sequence Synthetic oligonucleotide
tagggaagagaaggacatatgatttccagacgttattagcccgatctcctgtgtacgatccagttgactagtacatgaccactt

SEQ ID NO: 9 - DNA, 84 bp, Artificial Sequence
  Description of Artificial Sequence Synthetic oligonucleotide
tagggaagagaaggacatatgatgtatatgtaccaaccgagtgattcggcctatcaaagcgtcttgactagtacatgaccactt

SEQ ID NO: 10 - DNA, 84 bp, Artificial Sequence
  Description of Artificial Sequence Synthetic oligonucleotide
tagggaagagaaggacatatgatcgtttgggccctatgtacggcgtaaatttctcctgcagaattgactagtacatgaccactt

SEQ ID NO: 11 - DNA, 84 bp, Artificial Sequence
  Description of Artificial Sequence Synthetic oligonucleotide
tagggaagagaaggacatatgataaaaaaccggggttcttaattttcattgttcgtcgtacttttgactagtacatgaccactt

SEQ ID NO: 12 - DNA, 84 bp, Artificial Sequence
  Description of Artificial Sequence Synthetic oligonucleotide
tagggaagagaaggacatatgattacaagtaaaactgacccctccatttgtgtgtttattcgcttgactagtacatgaccactt

SEQ ID NO: 13 - DNA, 84 bp, Artificial Sequence
  Description of Artificial Sequence Synthetic oligonucleotide
tagggaagagaaggacatatgataccaatcatactaagcctacccgaaccagcggtggaatggttgactagtacatgaccactt

SEQ ID NO: 14 - DNA, 84 bp, Artificial Sequence
  Description of Artificial Sequence Synthetic oligonucleotide
tagggaagagaaggacatatgatcgtttgggccttacgtacggcgtaaatttctcctgcagaattgactagtacatgaccactt

SEQ ID NO: 15 - DNA, 84 bp, Artificial Sequence
  Description of Artificial Sequence Synthetic oligonucleotide
tagggaagagaaggacatatgatcgtttgggccttatgtacggcgtaaatttctcctgcagagttgactagtacatgaccactt

SEQ ID NO: 16 - DNA, 84 bp, Artificial Sequence
  Description of Artificial Sequence Synthetic oligonucleotide
tagggaagagaaggacatatgatttagtacgtattgatatgatctgaactatgtgagaatgagttgactagtacatgaccactt

SEQ ID NO: 17 - DNA, 84 bp, Artificial Sequence
  Description of Artificial Sequence Synthetic oligonucleotide
tagggaagagaaggacatatgatcgtttgggccttatgtacggcgtaaacttctcctgcagaattgactagtacatgaccactt

SEQ ID NO: 18 - DNA, 84 bp, Artificial Sequence
  Description of Artificial Sequence Synthetic oligonucleotide
tagggaagagaaggacatatgatccgtctattgccgaggatgggtaatagtaccgtgcgcactttgactagtacatgaccactt

SEQ ID NO: 19 - DNA, 84 bp, Artificial Sequence
  Description of Artificial Sequence Synthetic oligonucleotide
tagggaagagaaggacatatgatcgtttgggccttatgtacagcgtaaatttctcctgcagaattgactagtacatgaccactt

SEQ ID NO: 20 - DNA, 84 bp, Artificial Sequence
  Description of Artificial Sequence Synthetic oligonucleotide
tagggaagagaaggacatatgattgtttgggccttatgtacggcgtaaatttctcctgcagaattgactagtacatgaccactt

SEQ ID NO: 21 - DNA, 84 bp, Artificial Sequence
  Description of Artificial Sequence Synthetic oligonucleotide
tagggaagagaaggacatatgatcgtgagggagaccctacgagagagaaaagaaaaggaaaagttgactagtacatgaccactt

SEQ ID NO: 22 - DNA, 84 bp, Artificial Sequence
  Description of Artificial Sequence Synthetic oligonucleotide
tagggaagagaaggacatatgatcgtttgggccttatgtacggcgtaaatttctcctacagaattgactagtacatgaccactt

SEQ ID NO: 23 - DNA, 84 bp, Artificial Sequence
  Description of Artificial Sequence Synthetic oligonucleotide
tagggaagagaaggacatatgatatagaatgaggaggtcaccaatggacactaatcgaccgtattgactagtacatgaccactt

SEQ ID NO: 24 - DNA, 84 bp, Artificial Sequence
  Description of Artificial Sequence Synthetic oligonucleotide
tagggaagagaaggacatatgatggaggaaaagagacaacaaagaacgccgcgcacaaggcacttgactagtacatgaccactt

SEQ ID NO: 25 - DNA, 84 bp, Artificial Sequence
  Description of Artificial Sequence Synthetic oligonucleotide
tagggaagagaaggacatatgatcgttgatttacggtcgtaagcggtacggtttcatcgtcagttgactagtacatgaccactt

SEQ ID NO: 26 - DNA, 84 bp, Artificial Sequence
  Description of Artificial Sequence Synthetic oligonucleotide
tagggaagagaaggacatatgatcgtttgggccttatgtacggtgtaaatttctcctgcagaattgactagtacatgaccactt

SEQ ID NO: 27 - DNA, 84 bp, Artificial Sequence
  Description of Artificial Sequence Synthetic oligonucleotide
tagggaagagaaggacatatgatcgtttgggccttatgtacggcgtaaatttctcccgcagaattgactagtacatgaccactt

SEQ ID NO: 28 - DNA, 84 bp, Artificial Sequence
  Description of Artificial Sequence Synthetic oligonucleotide
tagggaagagaaggacatatgatcgtttgggccttatgtacggcgtaaatttcccctgcagaattgactagtacatgaccactt

SEQ ID NO: 29 - DNA, 84 bp, Artificial Sequence
  Description of Artificial Sequence Synthetic oligonucleotide
tagggaagagaaggacatatgatcgtttgggccttatgtacggcgtaaatttctcctgcaggattgactagtacatgaccactt

SEQ ID NO: 30 - DNA, 84 bp, Artificial Sequence
  Description of Artificial Sequence Synthetic oligonucleotide
tagggaagagaaggacatatgatcgagggtcctgttgactacgtctttgaactcattggtcacttgactagtacatgaccactt

SEQ ID NO: 31 - DNA, 84 bp, Artificial Sequence
  Description of Artificial Sequence Synthetic oligonucleotide
tagggaagagaaggacatatgatcgtttaggccttatgtacggcgtaaatttctcctgcagaattgactagtacatgaccactt

SEQ ID NO: 32 - DNA, 84 bp, Artificial Sequence
  Description of Artificial Sequence Synthetic oligonucleotide
tagggaagagaaggacatatgatcgtttgggcctcatgtacggcgtaaatttctcctgcagaattgactagtacatgaccactt

SEQ ID NO: 33 - DNA, 84 bp, Artificial Sequence
  Description of Artificial Sequence Synthetic oligonucleotide
tagggaagagaaggacatatgatggtctagaacagagataaccaacattgtcccgaaaagcccttgactagtacatgaccactt

SEQ ID NO: 34 - DNA, 84 bp, Artificial Sequence
  Description of Artificial Sequence Synthetic oligonucleotide
tagggaagagaaggacatatgattgctgatcccagcaaacggtatgacgcaacagaggtatcattgactagtacatgaccactt

SEQ ID NO: 35 - DNA, 84 bp, Artificial Sequence
  Description of Artificial Sequence Synthetic oligonucleotide
tagggaagagaaggacatatgatgagacaaatgatgtccatgcatgccgccaaacaaccgagattgactagtacatgaccactt

SEQ ID NO: 36 - DNA, 84 bp, Artificial Sequence
  Description of Artificial Sequence Synthetic oligonucleotide
tagggaagagaaggacatatgatccttcagaaaggaacatatgccgttaagaccagaacttcgttgactagtacatgaccactt

SEQ ID NO: 37 - DNA, 84 bp, Artificial Sequence
  Description of Artificial Sequence Synthetic oligonucleotide
tagggaagagaaggacatatgatcgtttgggccttatgcacggcgtaaatttctcctgcagaattgactagtacatgaccactt

SEQ ID NO: 38 - DNA, 84 bp, Artificial Sequence
  Description of Artificial Sequence Synthetic oligonucleotide
tagggaagagaaggacatatgatcacggaaacgtgtagaattacacgttaacgaagtgaggagttgactagtacatgaccactt

SEQ ID NO: 39 - DNA, 84 bp, Artificial Sequence
  Description of Artificial Sequence Synthetic oligonucleotide
tagggaagagaaggacatatgatcgtttgggccttgtgtacggcgtaaatttctcctgcagaattgactagtacatgaccactt

SEQ ID NO: 40 - DNA, 84 bp, Artificial Sequence
  Description of Artificial Sequence Synthetic oligonucleotide
tagggaagagaaggacatatgatcaaacgtcgaatacggattgtcaaacgaacaacaccgtatttgactagtacatgaccactt

SEQ ID NO: 41 - DNA, 84 bp, Artificial Sequence
  Description of Artificial Sequence Synthetic oligonucleotide
tagggaagagaaggacatatgatatggccatctgcctagtcatagattgggaatctgaaccgattgactagtacatgaccactt

SEQ ID NO: 42 - DNA, 84 bp, Artificial Sequence
  Description of Artificial Sequence Synthetic oligonucleotide
tagggaagagaaggacatatgatggcagtcagaaagctctcgagaatgtggacccgagagcagttgactagtacatgaccactt

SEQ ID NO: 43 - DNA, 84 bp, Artificial Sequence
  Description of Artificial Sequence Synthetic oligonucleotide
tagggaagagaaggacatatgatgccgtgtcgaggaacatcctagacaaggtgaaaagtcccattgactagtacatgaccactt

SEQ ID NO: 44 - DNA, 84 bp, Artificial Sequence
  Description of Artificial Sequence Synthetic oligonucleotide
tagggaagagaaggacatatgatcaagtagcatagtggacgaacgagcggaacaaatgcgtaattgactagtacatgaccactt

SEQ ID NO: 45 - DNA, 84 bp, Artificial Sequence
  Description of Artificial Sequence Synthetic oligonucleotide
tagggaagagaaggacatatgattgcgatgcgagtagaaaagcgtatgctacaggaacgtccattgactagtacatgaccactt

SEQ ID NO: 46 - DNA, 84 bp, Artificial Sequence
  Description of Artificial Sequence Synthetic oligonucleotide
tagggaagagaaggacatatgattttatcagagacgcgcccttagcaaacgtgttcttccgaattgactagtacatgaccactt

SEQ ID NO: 47 - DNA, 84 bp, Artificial Sequence
  Description of Artificial Sequence Synthetic oligonucleotide
tagggaagagaaggacatatgattatatggaatctccaatgtggcaacaggacacataaagaattgactagtacatgaccactt

SEQ ID NO: 48 - DNA, 84 bp, Artificial Sequence
  Description of Artificial Sequence Synthetic oligonucleotide
tagggaagagaaggacatatgatgccggatcaacccaaggagttgattagcatcattttacgattgactagtacatgaccactt

SEQ ID NO: 49 - DNA, 84 bp, Artificial Sequence
  Description of Artificial Sequence Synthetic oligonucleotide
tagggaagagaaggacatatgatgtaggggtccacgaagtgctaagaaggcacacattttgcgttgactagtacatgaccactt

SEQ ID NO: 50 - DNA, 84 bp, Artificial Sequence
  Description of Artificial Sequence Synthetic oligonucleotide
tagggaagagaaggacatatgataacagcgcgcgccctcaacgatagactataagtccaaaggttgactagtacatgaccactt

SEQ ID NO: 51 - DNA, 84 bp, Artificial Sequence
  Description of Artificial Sequence Synthetic oligonucleotide
tagggaagagaaggacatatgatgtacgcgtcagagtgtcgtgaacgacaaacgactgataccttgactagtacatgaccactt

SEQ ID NO: 52 - DNA, 84 bp, Artificial Sequence
  Description of Artificial Sequence Synthetic oligonucleotide
tagggaagagaaggacatatgatagtgataaaggattaaggaaatgatagtatcatagaaacattgactagtacatgaccactt

SEQ ID NO: 53 - DNA, 84 bp, Artificial Sequence
  Description of Artificial Sequence Synthetic oligonucleotide
tagggaagagaaggacatatgattaatgcactttgaacttaagctataataactgattagttgttgactagtacatgaccactt

SEQ ID NO: 54 - DNA, 84 bp, Artificial Sequence
  Description of Artificial Sequence Synthetic oligonucleotide
tagggaagagaaggacatatgatcgtttgggccttatgtacggcgtaaatctctcctgcagaattgactagtacatgaccactt

SEQ ID NO: 55 - DNA, 84 bp, Artificial Sequence
  Description of Artificial Sequence Synthetic oligonucleotide
tagggaagagaaggacatatgataatgcgacataccaatgtcggacgacaacaaggctaacatttgactagtacatgaccactt

SEQ ID NO: 56 - DNA, 84 bp, Artificial Sequence
  Description of Artificial Sequence Synthetic oligonucleotide
tagggaagagaaggacatatgataacgaaccgaatagacctgcgcgaagaaagggtctcagagttgactagtacatgaccactt

SEQ ID NO: 57 - DNA, 84 bp, Artificial Sequence
  Description of Artificial Sequence Synthetic oligonucleotide
tagggaagagaaggacatatgatcgagcgacacgaaaaggggcatgatcattgtccattgaagttgactagtacatgaccactt

SEQ ID NO: 58 - DNA, 84 bp, Artificial Sequence
  Description of Artificial Sequence Synthetic oligonucleotide
tagggaagagaaggacatatgataatgacaaacaaccgcctcacaggtttacggaacaagacattgactagtacatgaccactt

SEQ ID NO: 59 - DNA, 84 bp, Artificial Sequence
  Description of Artificial Sequence Synthetic oligonucleotide
tagggaagagaaggacatatgatcgtttgggccttatgtacggcgtaaatttctcctgtagaattgactagtacatgaccactt

SEQ ID NO: 60 - DNA, 84 bp, Artificial Sequence
  Description of Artificial Sequence Synthetic oligonucleotide
tagggaagagaaggacatatgattctatcggatccaacgcggatttgaatatcaagcgcaacgttgactagtacatgaccactt

SEQ ID NO: 61 - DNA, 84 bp, Artificial Sequence
  Description of Artificial Sequence Synthetic oligonucleotide
tagggaagagaaggacatatgatcagaaagtctgcgcagccagactgtgggtagacgacgccattgactagtacatgaccactt

SEQ ID NO: 62 - DNA, 84 bp, Artificial Sequence
  Description of Artificial Sequence Synthetic oligonucleotide
tagggaagagaaggacatatgatcgtttgagccttatgtacggcgtaaatttctcctgcagaattgactagtacatgaccactt

SEQ ID NO: 63 - DNA, 84 bp, Artificial Sequence
  Description of Artificial Sequence Synthetic oligonucleotide
tagggaagagaaggacatatgatgaggctagatggaccaagcctcctgatcatagtccgagagttgactagtacatgaccactt

SEQ ID NO: 64 - DNA, 84 bp, Artificial Sequence
  Description of Artificial Sequence Synthetic oligonucleotide
tagggaagagaaggacatatgataggcacagcctaggagattcctagattcccggaggcatctttgactagtacatgaccactt

SEQ ID NO: 65 - DNA, 84 bp, Artificial Sequence
  Description of Artificial Sequence Synthetic oligonucleotide
tagggaagagaaggacatatgatcgtagagatgaggcttgcattattcgttccaagcgatatgttgactagtacatgaccactt

SEQ ID NO: 66 - DNA, 84 bp, Artificial Sequence
  Description of Artificial Sequence Synthetic oligonucleotide
tagggaagagaaggacatatgatcgtttgggccttatgtgcggcgtaaatttctcctgcagaattgactagtacatgaccactt

SEQ ID NO: 67 - DNA, 84 bp, Artificial Sequence
  Description of Artificial Sequence Synthetic oligonucleotide
tagggaagagaaggacatatgatcgcttgggccttatgtacggcgtaaatttctcctgcagaattgactagtacatgaccactt

SEQ ID NO: 68 - DNA, 84 bp, Artificial Sequence
  Description of Artificial Sequence Synthetic oligonucleotide
tagggaagagaaggacatatgatcgcgaaacgaaaaggttagtcatcgcataggagaccgcccttgactagtacatgaccactt

SEQ ID NO: 69 - DNA, 84 bp, Artificial Sequence
  Description of Artificial Sequence Synthetic oligonucleotide
tagggaagagaaggacatatgatcgtctgggccttatgtacggcgtaaatttctcctgcagaattgactagtacatgaccactt

SEQ ID NO: 70 - DNA, 84 bp, Artificial Sequence
  Description of Artificial Sequence Synthetic oligonucleotide
tagggaagagaaggacatatgatgtgtaagagaaggaataaagtagcgctcaaggtaaagcaattgactagtacatgaccactt

SEQ ID NO: 71 - DNA, 84 bp, Artificial Sequence
  Description of Artificial Sequence Synthetic oligonucleotide
tagggaagagaaggacatatgatcagctgggcacgttgatcatagtacttcgatgcacggcgcttgactagtacatgaccactt

SEQ ID NO: 72 - DNA, 84 bp, Artificial Sequence
  Description of Artificial Sequence Synthetic oligonucleotide
tagggaagagaaggacatatgatcgtttgggccttatgtatggcgtaaatttctcctgcagaattgactagtacatgaccactt

SEQ ID NO: 73 - DNA, 84 bp, Artificial Sequence
  Description of Artificial Sequence Synthetic oligonucleotide
tagggaagagaaggacatatgattgcatggagacagacgcggagcgacctcggcacacatgaattgactagtacatgaccactt

SEQ ID NO: 74 - DNA, 84 bp, Artificial Sequence
  Description of Artificial Sequence Synthetic oligonucleotide
tagggaagagaaggacatatgatgcttctataaaagaagaacatagaacgcatcaattggaccttgactagtacatgaccactt

SEQ ID NO: 75 - DNA, 84 bp, Artificial Sequence
  Description of Artificial Sequence Synthetic oligonucleotide
tagggaagagaaggacatatgatgcgtgacagctaacacagaatgagagaggaaacgcactaattgactagtacatgaccactt

SEQ ID NO: 76 - DNA, 84 bp, Artificial Sequence
  Description of Artificial Sequence Synthetic oligonucleotide
tagggaagagaaggacatatgattcacctgaaatttcccaggctaaaatcatatggccaacagttgactagtacatgaccactt

SEQ ID NO: 77 - DNA, 84 bp, Artificial Sequence
  Description of Artificial Sequence Synthetic oligonucleotide
tagggaagagaaggacatatgatcgcaacgtggcagtatacagaaaaccgatgcgaatgtgtcttgactagtacatgaccactt

SEQ ID NO: 78 - DNA, 84 bp, Artificial Sequence
  Description of Artificial Sequence Synthetic oligonucleotide
tagggaagagaaggacatatgatcgtttgggccttatgtacggcgtaaatttctcctgcggaattgactagtacatgaccactt

SEQ ID NO: 79 - DNA, 84 bp, Artificial Sequence
  Description of Artificial Sequence Synthetic oligonucleotide
tagggaagagaaggacatatgataacgacaaccgagaagtagcgaaagacaaacaacttctcattgactagtacatgaccactt

SEQ ID NO: 80 - DNA, 84 bp, Artificial Sequence
  Description of Artificial Sequence Synthetic oligonucleotide
tagggaagagaaggacatatgatcggggctggcaaaaaacacaggaccgatcgttgtctctggttgactagtacatgaccactt

SEQ ID NO: 81 - DNA, 84 bp, Artificial Sequence
  Description of Artificial Sequence Synthetic oligonucleotide
tagggaagagaaggacatatgattcataaaaggtcaattgcagatctagtctgcagtgacttattgactagtacatgaccactt

SEQ ID NO: 82 - DNA, 84 bp, Artificial Sequence
  Description of Artificial Sequence Synthetic oligonucleotide
tagggaagagaaggacatatgatggcatcatgttcttcggccaagtttcgcttgcaaacctttttgactagtacatgaccactt

SEQ ID NO: 83 - DNA, 84 bp, Artificial Sequence
  Description of Artificial Sequence Synthetic oligonucleotide
tagggaagagaaggacatatgatcgtttgggccttatgtacggcgtgaatttctcctgcagaattgactagtacatgaccactt

SEQ ID NO: 84 - DNA, 84 bp, Artificial Sequence
  Description of Artificial Sequence Synthetic oligonucleotide
tagggaagagaaggacatatgatagacgacagaggagcctatcagctgccaatgactagtgacttgactagtacatgaccactt

SEQ ID NO: 85 - DNA, 84 bp, Artificial Sequence
  Description of Artificial Sequence Synthetic oligonucleotide
tagggaagagaaggacatatgattgcgtttgttgacactccttttcaaggatgcgttgtcaccttgactagtacatgaccactt

SEQ ID NO: 86 - DNA, 84 bp, Artificial Sequence
  Description of Artificial Sequence Synthetic oligonucleotide
tagggaagagaaggacatatgatgtatggagtccggggaaacggagtccaaagcgaatcccatttgactagtacatgaccactt

SEQ ID NO: 87 - DNA, 84 bp, Artificial Sequence
  Description of Artificial Sequence Synthetic oligonucleotide
tagggaagagaaggacatatgatcggtgacgcacgcaggattccaaggtctctgccaaatctattgactagtacatgaccactt

SEQ ID NO: 88 - DNA, 84 bp, Artificial Sequence
  Description of Artificial Sequence Synthetic oligonucleotide
tagggaagagaaggacatatgatgcagaaggatgaaagagcacgaatccaacgataattgaaattgactagtacatgaccactt

SEQ ID NO: 89 - DNA, 84 bp, Artificial Sequence
  Description of Artificial Sequence Synthetic oligonucleotide
tagggaagagaaggacatatgatactcaacaaaagaggaaatcgattaagacgcgacatacgtttgactagtacatgaccactt

SEQ ID NO: 90 - DNA, 84 bp, Artificial Sequence
  Description of Artificial Sequence Synthetic oligonucleotide
tagggaagagaaggacatatgatcaaaaacgtaaggatacagtaacacatatgtagaggttatttgactagtacatgaccactt

SEQ ID NO: 91 - DNA, 84 bp, Artificial Sequence
  Description of Artificial Sequence Synthetic oligonucleotide
tagggaagagaaggacatatgatcgtgctgaattagtgagtggtacacaccgccagcatgattttgactagtacatgaccactt

SEQ ID NO: 92 - DNA, 84 bp, Artificial Sequence
  Description of Artificial Sequence Synthetic oligonucleotide
tagggaagagaaggacatatgatgtgtaaaggaaatgtggaccacacaaccgcatttccgaaattgactagtacatgaccactt

SEQ ID NO: 93 - DNA, 84 bp, Artificial Sequence
  Description of Artificial Sequence Synthetic oligonucleotide
tagggaagagaaggacatatgatgccgcgagaagccaacgaccactcagtcgattggtagggattgactagtacatgaccactt

SEQ ID NO: 94 - DNA, 84 bp, Artificial Sequence
  Description of Artificial Sequence Synthetic oligonucleotide
tagggaagagaaggacatatgatgacgaagtgttgaaagagaagggcacccaaacactatcaattgactagtacatgaccactt

SEQ ID NO: 95 - DNA, 84 bp, Artificial Sequence
  Description of Artificial Sequence Synthetic oligonucleotide
tagggaagagaaggacatatgatccggacaggtcgaatcaactgatcaaggccggacttactgttgactagtacatgaccactt

SEQ ID NO: 96 - DNA, 84 bp, Artificial Sequence
  Description of Artificial Sequence Synthetic oligonucleotide
tagggaagagaaggacatatgatggacacaaaggtagagacctaggatatggtctcaagccagttgactagtacatgaccactt

SEQ ID NO: 97 - DNA, 84 bp, Artificial Sequence
  Description of Artificial Sequence Synthetic oligonucleotide
tagggaagagaaggacatatgatgccgaacaacaaattgggcggcaaataaaaaggatttccgttgactagtacatgaccactt

SEQ ID NO: 98 - DNA, 84 bp, Artificial Sequence
  Description of Artificial Sequence Synthetic oligonucleotide
tagggaagagaaggacatatgatgagatcgcaaaatgatgataacgaacttagcaatcgctaattgactagtacatgaccactt

SEQ ID NO: 99 - DNA, 84 bp, Artificial Sequence
  Description of Artificial Sequence Synthetic oligonucleotide
tagggaagagaaggacatatgatacgcgcggccctagcacgcaaacagtgagacaaagaatacttgactagtacatgaccactt

SEQ ID NO: 100 - DNA, 84 bp, Artificial Sequence
  Description of Artificial Sequence Synthetic oligonucleotide
tagggaagagaaggacatatgatttaacagccgcaatgaatatacaggcgtataaacatctcattgactagtacatgaccactt

SEQ ID NO: 101 - DNA, 40 bp, Artificial Sequence
  Description of Artificial Sequence Synthetic oligonucleotide
cgtttgggccttatgtacggcgtaaatttctcctgcagaa

SEQ ID NO: 102 - DNA, 40 bp, Artificial Sequence
  Description of Artificial Sequence Synthetic oligonucleotide
ccacttatctagcgtagataaggcgtttaaaaggtctaac

SEQ ID NO: 103 - DNA, 40 bp, Artificial Sequence
  Description of Artificial Sequence Synthetic oligonucleotide
ttcggtctagcacactcaacgagatactggggttaaacgt

SEQ ID NO: 104 - DNA, 40 bp, Artificial Sequence
  Description of Artificial Sequence Synthetic oligonucleotide
cccgatactgaaaatggaggcccgcaagtattatttacaa

SEQ ID NO: 105 - DNA, 40 bp, Artificial Sequence
  Description of Artificial Sequence Synthetic oligonucleotide
cacttcatgtaagactaaaagatggagcgtgaaggatgca

SEQ ID NO: 106 - DNA, 40 bp, Artificial Sequence
  Description of Artificial Sequence Synthetic oligonucleotide
gtgggttaactaatgaggcttaacgaggcgtcaacgtttt

SEQ ID NO: 107 - DNA, 40 bp, Artificial Sequence
  Description of Artificial Sequence Synthetic oligonucleotide
cgacactgtttgtaagtcttccctgattacttatttcatc

SEQ ID NO: 108 - DNA, 40 bp, Artificial Sequence
  Description of Artificial Sequence Synthetic oligonucleotide
ttccagacgttattagcccgatctcctgtgtacgatccag

SEQ ID NO: 109 - DNA, 40 bp, Artificial Sequence
  Description of Artificial Sequence Synthetic oligonucleotide
gtatatgtaccaaccgagtgattcggcctatcaaagcgtc

SEQ ID NO: 110 - DNA, 40 bp, Artificial Sequence
  Description of Artificial Sequence Synthetic oligonucleotide
cgtttgggccctatgtacggcgtaaatttctcctgcagaa

SEQ ID NO: 111 - DNA, 40 bp, Artificial Sequence
  Description of Artificial Sequence Synthetic oligonucleotide
aaaaaaccggggttcttaattttcattgttcgtcgtactt

SEQ ID NO: 112 - DNA, 40 bp, Artificial Sequence
  Description of Artificial Sequence Synthetic oligonucleotide
tacaagtaaaactgacccctccatttgtgtgtttattcgc

SEQ ID NO: 113 - DNA, 40 bp, Artificial Sequence
  Description of Artificial Sequence Synthetic oligonucleotide
accaatcatactaagcctacccgaaccagcggtggaatgg

SEQ ID NO: 114 - DNA, 40 bp, Artificial Sequence
  Description of Artificial Sequence Synthetic oligonucleotide
cgtttgggccttacgtacggcgtaaatttctcctgcagaa

SEQ ID NO: 115 - DNA, 40 bp, Artificial Sequence
  Description of Artificial Sequence Synthetic oligonucleotide
cgtttgggccttatgtacggcgtaaatttctcctgcagag

SEQ ID NO: 116 - DNA, 40 bp, Artificial Sequence
  Description of Artificial Sequence Synthetic oligonucleotide
ttagtacgtattgatatgatctgaactatgtgagaatgag

SEQ ID NO: 117 - DNA, 40 bp, Artificial Sequence
  Description of Artificial Sequence Synthetic oligonucleotide
cgtttgggccttatgtacggcgtaaacttctcctgcagaa

SEQ ID NO: 118 - DNA, 40 bp, Artificial Sequence
  Description of Artificial Sequence Synthetic oligonucleotide
ccgtctattgccgaggatgggtaatagtaccgtgcgcact

SEQ ID NO: 119 - DNA, 40 bp, Artificial Sequence
  Description of Artificial Sequence Synthetic oligonucleotide
cgtttgggccttatgtacagcgtaaatttctcctgcagaa

SEQ ID NO: 120 - DNA, 40 bp, Artificial Sequence
  Description of Artificial Sequence Synthetic oligonucleotide
tgtttgggccttatgtacggcgtaaatttctcctgcagaa

SEQ ID NO: 121 - DNA, 40 bp, Artificial Sequence
  Description of Artificial Sequence Synthetic oligonucleotide
cgtgagggagaccctacgagagagaaaagaaaaggaaaag

SEQ ID NO: 122 - DNA, 40 bp, Artificial Sequence
  Description of Artificial Sequence Synthetic oligonucleotide
cgtttgggccttatgtacggcgtaaatttctcctacagaa

SEQ ID NO: 123 - DNA, 40 bp, Artificial Sequence
  Description of Artificial Sequence Synthetic oligonucleotide
atagaatgaggaggtcaccaatggacactaatcgaccgta

SEQ ID NO: 124 - DNA, 40 bp, Artificial Sequence
  Description of Artificial Sequence Synthetic oligonucleotide
ggaggaaaagagacaacaaagaacgccgcgcacaaggcac

SEQ ID NO: 125 - DNA, 40 bp, Artificial Sequence
  Description of Artificial Sequence Synthetic oligonucleotide
cgttgatttacggtcgtaagcggtacggtttcatcgtcag

SEQ ID NO: 126 - DNA, 40 bp, Artificial Sequence
  Description of Artificial Sequence Synthetic oligonucleotide
cgtttgggccttatgtacggtgtaaatttctcctgcagaa

SEQ ID NO: 127 - DNA, 40 bp, Artificial Sequence
  Description of Artificial Sequence Synthetic oligonucleotide
cgtttgggccttatgtacggcgtaaatttctcccgcagaa

SEQ ID NO: 128 - DNA, 40 bp, Artificial Sequence
  Description of Artificial Sequence Synthetic oligonucleotide
cgtttgggccttatgtacggcgtaaatttcccctgcagaa

SEQ ID NO: 129 - DNA, 40 bp, Artificial Sequence
  Description of Artificial Sequence Synthetic oligonucleotide
cgtttgggccttatgtacggcgtaaatttctcctgcagga

SEQ ID NO: 130 - DNA, 40 bp, Artificial Sequence
  Description of Artificial Sequence Synthetic oligonucleotide
cgagggtcctgttgactacgtctttgaactcattggtcac

SEQ ID NO: 131 - DNA, 40 bp, Artificial Sequence
  Description of Artificial Sequence Synthetic oligonucleotide
cgtttaggccttatgtacggcgtaaatttctcctgcagaa

SEQ ID NO: 132 - DNA, 40 bp, Artificial Sequence
  Description of Artificial Sequence Synthetic oligonucleotide
cgtttgggcctcatgtacggcgtaaatttctcctgcagaa

SEQ ID NO: 133 - DNA, 40 bp, Artificial Sequence
  Description of Artificial Sequence Synthetic oligonucleotide
ggtctagaacagagataaccaacattgtcccgaaaagccc

SEQ ID NO: 134 - DNA, 40 bp, Artificial Sequence
  Description of Artificial Sequence Synthetic oligonucleotide
tgctgatcccagcaaacggtatgacgcaacagaggtatca

SEQ ID NO: 135 - DNA, 40 bp, Artificial Sequence
  Description of Artificial Sequence Synthetic oligonucleotide
gagacaaatgatgtccatgcatgccgccaaacaaccgaga

SEQ ID NO: 136 - DNA, 40 bp, Artificial Sequence
  Description of Artificial Sequence Synthetic oligonucleotide
ccttcagaaaggaacatatgccgttaagaccagaacttcg

SEQ ID NO: 137 - DNA, 40 bp, Artificial Sequence
  Description of Artificial Sequence Synthetic oligonucleotide
cgtttgggccttatgcacggcgtaaatttctcctgcagaa

SEQ ID NO: 138 - DNA, 40 bp, Artificial Sequence
  Description of Artificial Sequence Synthetic oligonucleotide
cacggaaacgtgtagaattacacgttaacgaagtgaggag

SEQ ID NO: 139 - DNA, 40 bp, Artificial Sequence
  Description of Artificial Sequence Synthetic oligonucleotide
cgtttgggccttgtgtacggcgtaaatttctcctgcagaa

SEQ ID NO: 140 - DNA, 40 bp, Artificial Sequence
  Description of Artificial Sequence Synthetic oligonucleotide
caaacgtcgaatacggattgtcaaacgaacaacaccgtat

SEQ ID NO: 141 - DNA, 40 bp, Artificial Sequence
  Description of Artificial Sequence Synthetic oligonucleotide
atggccatctgcctagtcatagattgggaatctgaaccga

SEQ ID NO: 142 - DNA, 40 bp, Artificial Sequence
  Description of Artificial Sequence Synthetic oligonucleotide
ggcagtcagaaagctctcgagaatgtggacccgagagcag

SEQ ID NO: 143 - DNA, 40 bp, Artificial Sequence
  Description of Artificial Sequence Synthetic oligonucleotide
gccgtgtcgaggaacatcctagacaaggtgaaaagtccca

SEQ ID NO: 144 - DNA, 40 bp, Artificial Sequence
  Description of Artificial Sequence Synthetic oligonucleotide
caagtagcatagtggacgaacgagcggaacaaatgcgtaa

SEQ ID NO: 145 - DNA, 40 bp, Artificial Sequence
  Description of Artificial Sequence Synthetic oligonucleotide
tgcgatgcgagtagaaaagcgtatgctacaggaacgtcca

SEQ ID NO: 146 - DNA, 40 bp, Artificial Sequence
  Description of Artificial Sequence Synthetic oligonucleotide
tttatcagagacgcgcccttagcaaacgtgttcttccgaa

SEQ ID NO: 147 - DNA, 40 bp, Artificial Sequence
  Description of Artificial Sequence Synthetic oligonucleotide
tatatggaatctccaatgtggcaacaggacacataaagaa

SEQ ID NO: 148 - DNA, 40 bp, Artificial Sequence
  Description of Artificial Sequence Synthetic oligonucleotide
gccggatcaacccaaggagttgattagcatcattttacga

SEQ ID NO: 149 - DNA, 40 bp, Artificial Sequence
  Description of Artificial Sequence Synthetic oligonucleotide
gtaggggtccacgaagtgctaagaaggcacacattttgcg

SEQ ID NO: 150 - DNA, 40 bp, Artificial Sequence
  Description of Artificial Sequence Synthetic oligonucleotide
aacagcgcgcgccctcaacgatagactataagtccaaagg

SEQ ID NO: 151 - DNA, 40 bp, Artificial Sequence
  Description of Artificial Sequence Synthetic oligonucleotide
gtacgcgtcagagtgtcgtgaacgacaaacgactgatacc

SEQ ID NO: 152 - DNA, 40 bp, Artificial Sequence
  Description of Artificial Sequence Synthetic oligonucleotide
agtgataaaggattaaggaaatgatagtatcatagaaaca

SEQ ID NO: 153 - DNA, 40 bp, Artificial Sequence
  Description of Artificial Sequence Synthetic oligonucleotide
taatgcactttgaacttaagctataataactgattagttg

SEQ ID NO: 154 - DNA, 40 bp, Artificial Sequence
  Description of Artificial Sequence Synthetic oligonucleotide
cgtttgggccttatgtacggcgtaaatctctcctgcagaa

SEQ ID NO: 155 - DNA, 40 bp, Artificial Sequence
  Description of Artificial Sequence Synthetic oligonucleotide
aatgcgacataccaatgtcggacgacaacaaggctaacat

SEQ ID NO: 156 - DNA, 40 bp, Artificial Sequence
  Description of Artificial Sequence Synthetic oligonucleotide
aacgaaccgaatagacctgcgcgaagaaagggtctcagag

SEQ ID NO: 157 - DNA, 40 bp, Artificial Sequence
  Description of Artificial Sequence Synthetic oligonucleotide
cgagcgacacgaaaaggggcatgatcattgtccattgaag

SEQ ID NO: 158 - DNA, 40 bp, Artificial Sequence
  Description of Artificial Sequence Synthetic oligonucleotide
aatgacaaacaaccgcctcacaggtttacggaacaagaca

SEQ ID NO: 159 - DNA, 40 bp, Artificial Sequence
  Description of Artificial Sequence Synthetic oligonucleotide
cgtttgggccttatgtacggcgtaaatttctcctgtagaa

SEQ ID NO: 160 - DNA, 40 bp, Artificial Sequence
  Description of Artificial Sequence Synthetic oligonucleotide
tctatcggatccaacgcggatttgaatatcaagcgcaacg

SEQ ID NO: 161 - DNA, 40 bp, Artificial Sequence
  Description of Artificial Sequence Synthetic oligonucleotide
cagaaagtctgcgcagccagactgtgggtagacgacgcca

SEQ ID NO: 162 - DNA, 40 bp, Artificial Sequence
  Description of Artificial Sequence Synthetic oligonucleotide
cgtttgagccttatgtacggcgtaaatttctcctgcagaa

SEQ ID NO: 163 - DNA, 40 bp, Artificial Sequence
  Description of Artificial Sequence Synthetic oligonucleotide
gaggctagatggaccaagcctcctgatcatagtccgagag

SEQ ID NO: 164 - DNA, 40 bp, Artificial Sequence
  Description of Artificial Sequence Synthetic oligonucleotide
aggcacagcctaggagattcctagattcccggaggcatct

SEQ ID NO: 165 - DNA, 40 bp, Artificial Sequence
  Description of Artificial Sequence Synthetic oligonucleotide
cgtagagatgaggcttgcattattcgttccaagcgatatg

SEQ ID NO: 166 - DNA, 40 bp, Artificial Sequence
  Description of Artificial Sequence Synthetic oligonucleotide
cgtttgggccttatgtgcggcgtaaatttctcctgcagaa

SEQ ID NO: 167 - DNA, 40 bp, Artificial Sequence
  Description of Artificial Sequence Synthetic oligonucleotide
cgcttgggccttatgtacggcgtaaatttctcctgcagaa

SEQ ID NO: 168 - DNA, 40 bp, Artificial Sequence
  Description of Artificial Sequence Synthetic oligonucleotide
cgcgaaacgaaaaggttagtcatcgcataggagaccgccc

SEQ ID NO: 169 - DNA, 40 bp, Artificial Sequence
  Description of Artificial Sequence Synthetic oligonucleotide
cgtctgggccttatgtacggcgtaaatttctcctgcagaa

SEQ ID NO: 170 - DNA, 40 bp, Artificial Sequence
  Description of Artificial Sequence Synthetic oligonucleotide
gtgtaagagaaggaataaagtagcgctcaaggtaaagcaa

SEQ ID NO: 171 - DNA, 40 bp, Artificial Sequence
  Description of Artificial Sequence Synthetic oligonucleotide
cagctgggcacgttgatcatagtacttcgatgcacggcgc

SEQ ID NO: 172 - DNA, 40 bp, Artificial Sequence
  Description of Artificial Sequence Synthetic oligonucleotide
cgtttgggccttatgtatggcgtaaatttctcctgcagaa

SEQ ID NO: 173 - DNA, 40 bp, Artificial Sequence
  Description of Artificial Sequence Synthetic oligonucleotide
tgcatggagacagacgcggagcgacctcggcacacatgaa

SEQ ID NO: 174 - DNA, 40 bp, Artificial Sequence
  Description of Artificial Sequence Synthetic oligonucleotide
gcttctataaaagaagaacatagaacgcatcaattggacc

SEQ ID NO: 175 - DNA, 40 bp, Artificial Sequence
  Description of Artificial Sequence Synthetic oligonucleotide
gcgtgacagctaacacagaatgagagaggaaacgcactaa

SEQ ID NO: 176 - DNA, 40 bp, Artificial Sequence
  Description of Artificial Sequence Synthetic oligonucleotide
tcacctgaaatttcccaggctaaaatcatatggccaacag

SEQ ID NO: 177 - DNA, 40 bp, Artificial Sequence
  Description of Artificial Sequence Synthetic oligonucleotide
cgcaacgtggcagtatacagaaaaccgatgcgaatgtgtc

SEQ ID NO: 178 - DNA, 40 bp, Artificial Sequence
  Description of Artificial Sequence Synthetic oligonucleotide
cgtttgggccttatgtacggcgtaaatttctcctgcggaa

SEQ ID NO: 179 - DNA, 40 bp, Artificial Sequence
  Description of Artificial Sequence Synthetic oligonucleotide
aacgacaaccgagaagtagcgaaagacaaacaacttctca

SEQ ID NO: 180 - DNA, 40 bp, Artificial Sequence
  Description of Artificial Sequence Synthetic oligonucleotide
cggggctggcaaaaaacacaggaccgatcgttgtctctgg

SEQ ID NO: 181 - DNA, 40 bp, Artificial Sequence
  Description of Artificial Sequence Synthetic oligonucleotide
tcataaaaggtcaattgcagatctagtctgcagtgactta

SEQ ID NO: 182 - DNA, 40 bp, Artificial Sequence
  Description of Artificial Sequence Synthetic oligonucleotide
ggcatcatgttcttcggccaagtttcgcttgcaaaccttt

SEQ ID NO: 183 - DNA, 40 bp, Artificial Sequence
  Description of Artificial Sequence Synthetic oligonucleotide
cgtttgggccttatgtacggcgtgaatttctcctgcagaa

SEQ ID NO: 184 - DNA, 40 bp, Artificial Sequence
  Description of Artificial Sequence Synthetic oligonucleotide
agacgacagaggagcctatcagctgccaatgactagtgac

SEQ ID NO: 185 - DNA, 40 bp, Artificial Sequence
  Description of Artificial Sequence Synthetic oligonucleotide
tgcgtttgttgacactccttttcaaggatgcgttgtcacc

SEQ ID NO: 186 - DNA, 40 bp, Artificial Sequence
  Description of Artificial Sequence Synthetic oligonucleotide
gtatggagtccggggaaacggagtccaaagcgaatcccat

SEQ ID NO: 187 - DNA, 40 bp, Artificial Sequence
  Description of Artificial Sequence Synthetic oligonucleotide
cggtgacgcacgcaggattccaaggtctctgccaaatcta

SEQ ID NO: 188 - DNA, 40 bp, Artificial Sequence
  Description of Artificial Sequence Synthetic oligonucleotide
gcagaaggatgaaagagcacgaatccaacgataattgaaa

SEQ ID NO: 189 - DNA, 40 bp, Artificial Sequence
  Description of Artificial Sequence Synthetic oligonucleotide
actcaacaaaagaggaaatcgattaagacgcgacatacgt

SEQ ID NO: 190 - DNA, 40 bp, Artificial Sequence
  Description of Artificial Sequence Synthetic oligonucleotide
caaaaacgtaaggatacagtaacacatatgtagaggttat

SEQ ID NO: 191 - DNA, 40 bp, Artificial Sequence
  Description of Artificial Sequence Synthetic oligonucleotide
cgtgctgaattagtgagtggtacacaccgccagcatgatt

SEQ ID NO: 192 - DNA, 40 bp, Artificial Sequence
  Description of Artificial Sequence Synthetic oligonucleotide
gtgtaaaggaaatgtggaccacacaaccgcatttccgaaa

SEQ ID NO: 193 - DNA, 40 bp, Artificial Sequence
  Description of Artificial Sequence Synthetic oligonucleotide
gccgcgagaagccaacgaccactcagtcgattggtaggga

SEQ ID NO: 194 - DNA, 40 bp, Artificial Sequence
  Description of Artificial Sequence Synthetic oligonucleotide
gacgaagtgttgaaagagaagggcacccaaacactatcaa

SEQ ID NO: 195 - DNA, 40 bp, Artificial Sequence
  Description of Artificial Sequence Synthetic oligonucleotide
ccggacaggtcgaatcaactgatcaaggccggacttactg

SEQ ID NO: 196 - DNA, 40 bp, Artificial Sequence
  Description of Artificial Sequence Synthetic oligonucleotide
ggacacaaaggtagagacctaggatatggtctcaagccag

SEQ ID NO: 197 - DNA, 40 bp, Artificial Sequence
  Description of Artificial Sequence Synthetic oligonucleotide
gccgaacaacaaattgggcggcaaataaaaaggatttccg

SEQ ID NO: 198 - DNA, 40 bp, Artificial Sequence
  Description of Artificial Sequence Synthetic oligonucleotide
gagatcgcaaaatgatgataacgaacttagcaatcgctaa

SEQ ID NO: 199 - DNA, 40 bp, Artificial Sequence
  Description of Artificial Sequence Synthetic oligonucleotide
acgcgcggccctagcacgcaaacagtgagacaaagaatac

SEQ ID NO: 200 - DNA, 40 bp, Artificial Sequence
  Description of Artificial Sequence Synthetic oligonucleotide
ttaacagccgcaatgaatatacaggcgtataaacatctca

SEQ ID NO: 201 - DNA, 23 bp, Artificial Sequence
  Description of Artificial Sequence Synthetic oligonucleotide
tagggaagagaaggacatatgat

SEQ ID NO: 202 - DNA, 21 bp, Artificial Sequence
  Description of Artificial Sequence Synthetic oligonucleotide
ttgactagtacatgaccactt